(19) United States
(12) Patent Application Publication
(10) Pub. No.: US 2022/0047870 A1
(43) Pub. Date: Feb. 17, 2022

(54) SYSTEM AND METHOD FOR NEURAL CONTROL

(71) Applicant: BIOS HEALTH LTD, Cambridge Cambridgeshire (GB)

(72) Inventors: Oliver ARMITAGE, Cambridge (GB); Emil Hewage, Cambridge (GB); Samuel Gonshaw, Cambridge (GB); Matjaz Jakopec, Cambridge (GB); Tristan Edwards, Cambridge (GB)

(21) Appl. No.: 17/384,651

(22) Filed: Jul. 23, 2021

Related U.S. Application Data
(63) Continuation of application No. PCT/EP2020/051832, filed on Jan. 24, 2020.

(30) Foreign Application Priority Data
Jan. 24, 2019 (GB) 1900995.0

(51) Int. Cl. A61N 1/36; G16H 40/67; G16H 50/20; G16H 50/70; G16H 20/30; G05B 13/02; G05B 6/02

(52) U.S. Cl. CPC A61N 1/36139; G16H 40/67; G16H 50/20; G05B 6/02; G16H 20/30; G05B 13/0265; G16H 50/70

(57) ABSTRACT

A neural control system comprises an input controller arranged to receive neural data regarding neural signals relating to a bodily state of a subject from at least one neural sensor, at least one machine learning means using at least one machine learning model to process the received neural data to determine at least one output signal required to achieve a desired value of the bodily state, and means arranged to send the determined output signal to at least one output device, whereby the neural control system forms a first control loop providing closed loop control of the bodily state.

### CROSS-REFERENCE TO RELATED APPLICATIONS

This application claims benefit under 35 U.S.C. 120 to International Patent Application No. PCT/EP2020/051832, filed on Jan. 24, 2020, entitled “SYSTEM AND METHOD FOR NEURAL CONTROL”, which claims priority to United Kingdom Application No. GB 1900995.0, filed on Jan. 24, 2019, both of which are expressly incorporated by reference herein in their entireties.

### BACKGROUND

The present application relates to a system and method for neural control, and in particular to a system and method for closed loop neural control.

There are a range of implanted active electronic devices which are implanted in the human or animal body to stimulate electrophysiological targets, such as nerves and/or muscles, in order to provide treatment or palliation of a number of different conditions.

Examples of such implanted active electronic devices include various kinds of implantable pulse generators (IPGs) and neuromodulation devices that stimulate targets such as the heart muscle to regulate heart beat (pacemakers), the spinal cord to block pain signals (Spinal Cord Stimulators—SCS), regions of the brain to alter brain function such as reducing depression or stopping an epileptic seizure (Deep Brain Stimulator—DBS). These devices are currently used to provide treatment across a range of conditions, including spinal cord stimulation (SCS) for chronic pain, deep brain stimulation (DBS) for essential tremor, epilepsy, depression and obesity, vagal nerve stimulation for rheumatoid arthritis and Crohn's disease, among others. These current devices are currently used to provide treatment for more than half a million patients worldwide.

Currently these treatments are almost entirely provided by devices which provide stimulation without any responsiveness to local activity such as bodily variables that provide information about a target condition, or local neural activity encoding information about the brain's control of the target condition. For example treatment for hypertension (high blood pressure) may be provided by devices which provide stimulation at predetermined times without any responsiveness measured blood pressure or local neural activity encoding information about the brain's control of blood pressure.

It is desirable to be able to detect and process information encoding full neural activity in real time, or close to real time, and use this full neural activity to control implanted electronic devices that stimulate electrophysiological targets within a human or animal body in order to provide improved effectiveness.

### SUMMARY

The embodiments described below are not limited to implementations which solve any or all of the disadvantages of the known approaches described above.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter; variants and alternative features which facilitate the working of the invention and/or serve to achieve a substantially similar technical effect should be considered as falling into the scope of the invention disclosed herein.

In a first aspect, the present disclosure provides a neural control system comprising: an input controller arranged to receive neural data regarding neural signals relating to a bodily state of a subject from at least one neural sensor; at least one machine learning means using at least one machine learning model to process the received neural data to determine at least one output signal required to achieve a desired value of the bodily state; and means arranged to send the determined output signal to at least one output device; whereby the neural control system forms a first control loop providing closed loop control of the bodily state.

In a second aspect, the present disclosure provides a neural control system comprising: an input controller arranged to receive neural data regarding neural signals relating to a bodily state from at least one neural sensor; at least one machine learning model for processing the received neural data to identify neural biomarkers; and means arranged to send the identified neural biomarkers to at least one output device for performing operations; wherein the neural control system forms a first control loop providing closed loop control of the bodily state.

In a third aspect, the present disclosure provides a neural control system comprising: an input controller arranged to receive neural data regarding neural signals relating to a bodily state in nerves of a subject from at least one neural sensor; at least one neural data processing means arranged to process the received neural data to determine at least one output signal required to achieve a desired value of the bodily state; and an output controller arranged to receive the determined output signal and to send the determined output signal to the at least one output device; whereby the neural control system forms a first control loop providing closed loop control of the bodily state; wherein the output controller is further arranged to receive selected data regarding timing of received neural data from the input controller; and wherein the output controller is arranged to control the time at which the determined output signal is sent to the at least one output device based on the selected data regarding timing of received neural data.

In a fourth aspect, the present disclosure provides a neural control system comprising: an input controller arranged to receive neural data regarding neural signals relating to a bodily state in nerves of a subject from at least one neural sensor; at least one neural data processing means arranged to process the received neural data to determine at least one output signal required to achieve a desired value of the bodily state; and an output controller arranged to receive the determined output signal and to send the determined output signal to the at least one output device; whereby the neural control system forms a first control loop providing closed loop control of the bodily state; wherein the output controller is further arranged to receive selected data regarding amplitude of received neural data from the input controller; and wherein the output controller is arranged to control the amplitude of the determined output signal sent to the at least one output device based on the selected data regarding amplitude of received neural data.

In a fifth aspect, the present disclosure provides a neural control system comprising: an input controller arranged to receive neural data regarding neural signals relating to a bodily state in nerves of a subject from at least one neural sensor; at least one neural data processing means arranged to process the received neural data to determine at least one output neural stimulation signal required to achieve a desired value of the bodily state; and an output controller arranged to receive the determined output neural stimulation signal and to send the determined output neural stimulation signal to the at least one output device; whereby the neural control system forms a first control loop providing closed loop control of the bodily state; wherein the output controller is further arranged to receive selected data regarding amplitude of received neural data from the input controller; and wherein the output controller is further arranged to send data regarding the timing of the determined output neural stimulation signal to the input controller; and the input controller is arranged to stop sending the received neural data to the at least one machine learning means when the determined output neural stimulation signal is being output.

In a sixth aspect, the present disclosure provides a neural control system comprising: an input controller arranged to receive neural data regarding neural signals relating to a bodily state from at least one neural sensor; at least one machine learning means using at least one machine learning model to process the received neural data to determine at least one output signal required to achieve a desired value of the bodily state; and means arranged to send the determined output signal to at least one output device; whereby the neural control system forms a first control loop providing closed loop control of the bodily state; and wherein the neural control system is connected to a separate update machine, including means for sending the received neural data to the update machine; wherein the update machine is arranged to use the received neural data to perform machine learning training to produce an updated machine learning model; and the neural control system is further arranged to replace the at least one machine learning model with the updated machine learning model.

In a seventh aspect, the present disclosure provides a neural control system comprising: an input controller arranged to receive neural data regarding neural signals relating to a bodily state from at least one neural sensor; at least one neural data processing means arranged to process the received neural data to determine at least one output signal required to achieve a desired value of the bodily state; and means arranged to send the determined output signal to at least one output device; whereby the neural control system forms a first control loop providing closed loop control of the bodily state; and wherein the neural control system incorporates a safety module monitoring performance of the closed loop controller; wherein the safety module is arranged to act to reduce or stop the function of any element of the first control loop based on monitoring its performance

In an eighth aspect, the present disclosure provides a neural control system comprising: an input controller arranged to receive neural data regarding neural signals relating to a bodily state from at least one neural sensor; at least one neural data processing means arranged to process the received neural data to determine at least one output signal required to achieve a desired value of the bodily state; and means arranged to send the determined output signal to at least one output device; whereby the neural control system forms a first control loop providing closed loop control of the bodily state; and wherein the neural control system further comprises an auditing module arranged to monitor version control of the neural control system; wherein the auditing module is arranged to receive information from the update machine regarding the correct version of the neural control system; wherein the auditing module is arranged to obtain information regarding the current version of the neural control system; wherein the auditing module is arranged to command a safety module to act to reduce or stop the function of the neural control system based on the correct and current version of the neural control system.

In a ninth aspect, the present disclosure provides a neural control system comprising: an input controller arranged to receive neural data regarding neural signals relating to a bodily state from at least one neural sensor; at least one neural data processing means arranged to process the received neural data to determine at least one output signal required to achieve a desired value of the bodily state; and means arranged to send the determined output signal to at least one output device; whereby the neural control system forms a first control loop providing closed loop control of the bodily state; and wherein the desired value of bodily state is achieved by a bodily setpoint set in advance as a treatment.

In a tenth aspect, the present disclosure provides a neural control system comprising: an input controller arranged to receive neural data regarding neural signals relating to a bodily state from at least one neural sensor; at least one neural data processing means arranged to process the received neural data to determine at least one output signal required to achieve a desired value of the bodily state; and means arranged to send the determined output signal to at least one output device; whereby the neural control system forms a first control loop providing closed loop control of the bodily state; wherein the desired value of bodily state is achieved by a bodily setpoint; and wherein the setpoint is calculated within the closed loop controller based on received sensor data.

In an eleventh aspect, the present disclosure provides a neural control system comprising: an input controller arranged to receive neural data regarding neural signals relating to a bodily state from at least one neural sensor; at least one neural data processing means arranged to process the received neural data to determine at least one output signal required to achieve a desired value of the bodily state; and means arranged to send the determined output signal to at least one output device; whereby the neural control system forms a first control loop providing closed loop control of the bodily state; and wherein the closed loop controller uses the output of a body model to inform the decision of desired change in bodily state; wherein the current state of the body relative to the body model is informed by any combination of neural biomarkers or non-neural sensors.

In a twelfth aspect, the present disclosure provides a neural control method comprising: receiving neural data regarding neural signals relating to a bodily state of a subject from at least one neural sensor; using at least one machine learning model to process the received neural data to determine at least one output signal required to achieve a desired value of the bodily state; and sending the determined output signal to at least one output device; whereby the method comprises a first control loop providing closed loop control of the bodily state.

In a thirteenth aspect, the present disclosure provides a neural control method comprising: using an input controller to receive neural data regarding neural signals relating to a bodily state from at least one neural sensor; using at least one machine learning model for processing the received neural data to identify neural biomarkers; and sending the identified neural biomarkers to at least one output device for performing operations; wherein the neural control method forms a first control loop providing closed loop control of the bodily state.

In a fourteenth aspect, the present disclosure provides a neural control method comprising: using an input controller to receive neural data regarding neural signals relating to a bodily state in nerves of a subject from at least one neural sensor; using at least one neural data processing means to process the received neural data to determine at least one output signal required to achieve a desired value of the bodily state; and using an output controller to receive the determined output signal and to send the determined output signal to the at least one output device; whereby the neural control system forms a first control loop providing closed loop control of the bodily state; wherein the output controller is further used to receive selected data regarding timing of received neural data from the input controller; and wherein the output controller is used to control the time at which the determined output signal is sent to the at least one output device based on the selected data regarding timing of received neural data.

In a fifteenth aspect, the present disclosure provides a neural control method comprising: using an input controller to receive neural data regarding neural signals relating to a bodily state in nerves of a subject from at least one neural sensor; using at least one neural data processing means to process the received neural data to determine at least one output signal required to achieve a desired value of the bodily state; and using an output controller to receive the determined output signal and to send the determined output signal to the at least one output device; whereby the neural control method forms a first control loop providing closed loop control of the bodily state; wherein the output controller receives selected data regarding amplitude of received neural data from the input controller; and wherein the output controller controls the amplitude of the determined output signal sent to the at least one output device based on the selected data regarding amplitude of received neural data.

In a sixteenth aspect, the present disclosure provides a neural control method comprising: using an input controller to receive neural data regarding neural signals relating to a bodily state in nerves of a subject from at least one neural sensor; using at least one neural data processing means to process the received neural data to determine at least one output neural stimulation signal required to achieve a desired value of the bodily state; and using an output controller to receive the determined output neural stimulation signal and to send the determined output neural stimulation signal to the at least one output device; whereby the neural control method forms a first control loop providing closed loop control of the bodily state; wherein the output controller receives selected data regarding amplitude of received neural data from the input controller; and wherein the output controller sends data regarding the timing of the determined output neural stimulation signal to the input controller; and the input controller stops sending the received neural data to the at least one machine learning means when the determined output neural stimulation signal is being output.

In a seventeenth aspect, the present disclosure provides a neural control method comprising: using an input controller to receive neural data regarding neural signals relating to a bodily state from at least one neural sensor; using at least one machine learning means using at least one machine learning model to process the received neural data to determine at least one output signal required to achieve a desired value of the bodily state; and sending the determined output signal to at least one output device; whereby the neural control method forms a first control loop providing closed loop control of the bodily state; and wherein the neural control method further comprises using an update machine, including sending the received neural data to the update machine; wherein the update machine uses the received neural data to perform machine learning training to produce an updated neural control method; and the neural control method further comprising replacing the at least one neural control method with the updated neural control method.

In an eighteenth aspect, the present disclosure provides a neural control method comprising: using an input controller to receive neural data regarding neural signals relating to a bodily state from at least one neural sensor; using at least one neural data processing means to process the received neural data to determine at least one output signal required to achieve a desired value of the bodily state; and sending the determined output signal to at least one output device; whereby the neural control method forms a first control loop providing closed loop control of the bodily state; and wherein the neural control method uses a safety module monitoring performance of the closed loop controller; wherein the safety module acts to reduce or stop the function of any element of the first control loop based on monitoring its performance.

In an nineteenth aspect, the present disclosure provides a neural control method comprising: using an input controller to receive neural data regarding neural signals relating to a bodily state from at least one neural sensor; using at least one neural data processing means to process the received neural data to determine at least one output signal required to achieve a desired value of the bodily state; and sending the determined output signal to at least one output device; whereby the neural control method forms a first control loop providing closed loop control of the bodily state; and wherein the neural control method further comprises using an auditing module to monitor version control of the neural control system; wherein the auditing module receives information from the update machine regarding the correct version of the neural control system; wherein the auditing module obtains information regarding the current version of the neural control system; and wherein the auditing module commands a safety module to act to reduce or stop the function of the neural control system based on the correct and current version of the neural control system.

In a twentieth aspect, the present disclosure provides a neural control method comprising: using an input controller to receive neural data regarding neural signals relating to a bodily state from at least one neural sensor; using at least one neural data processing means to process the received neural data to determine at least one output signal required to achieve a desired value of the bodily state; and sending the determined output signal to at least one output device; whereby the neural control method forms a first control loop providing closed loop control of the bodily state; and wherein the desired value of bodily state is achieved by a bodily setpoint set in advance as a treatment.

In a twenty-first aspect, the present disclosure provides a neural control method comprising: using an input controller to receive neural data regarding neural signals relating to a bodily state from at least one neural sensor; using at least one neural data processing means to process the received neural data to determine at least one output signal required to achieve a desired value of the bodily state; and sending the determined output signal to at least one output device; whereby the neural control method forms a first control loop providing closed loop control of the bodily state; wherein the desired value of bodily state is achieved by a bodily setpoint; and wherein the setpoint is calculated within the closed loop controller based on received sensor data.

In a twenty-second aspect, the present disclosure provides a neural control method comprising: using an input controller to receive neural data regarding neural signals relating to a bodily state from at least one neural sensor; using at least one neural data processing means to process the received neural data to determine at least one output signal required to achieve a desired value of the bodily state; and sending the determined output signal to at least one output device; whereby the neural control system forms a first control loop providing closed loop control of the bodily state; and wherein the closed loop controller uses the output of a body model to inform the decision of desired change in bodily state; wherein the current state of the body relative to the body model is informed by any combination of neural biomarkers or non-neural sensors.

In a twenty-third aspect, the present disclosure provides a computer program comprising instructions which, when executed on a processing device, causes the processing device to carry out a method according to any of the twelfth to twenty-second aspects

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The features of each of the above aspects and/or embodiments may be combined as appropriate, as would be apparent to the skilled person, and may be combined with any of the aspects of the invention. Indeed, the order of the embodiments and the ordering and location of the preferable features is indicative only and has no bearing on the features themselves. It is intended for each of the preferable and/or optional features to be interchangeable and/or combinable with not only all of the aspect and embodiments, but also each of preferable features.

### BRIEF DESCRIPTION OF THE DRAWINGS

Embodiments of the invention will be described, by way of example, with reference to the following drawings, in which:

FIG. 1 is an explanatory diagram of a software architecture of a neural control system according to an example;

FIG. 2a is a diagram of a first example of operation of the neural control system of FIG. 1;

FIG. 2b is a diagram of a second example of operation of the neural control system of FIG. 1;

FIG. 2c is a diagram of a third example of operation of the neural control system of FIG. 1;

FIG. 3 is a representation of an example of data flows in the neural control system of FIG. 1;

FIG. 4 is a diagram of a fourth example of operation of the neural control system of FIG. 1;

FIG. 5 is a schematic diagram of a first example of a control system useable in the system of FIG. 1;

FIG. 6 is a schematic diagram of a second example of a control system useable in the system of FIG. 1;

FIG. 7 is a schematic diagram of a first example of a configuration in which the system of FIG. 1 can be used;

FIG. 8 is a schematic diagram of a second example of a configuration in which the system of FIG. 1 can be used; and

FIG. 9 is a schematic diagram of a third example of a configuration in which the system of FIG. 1 can be used.

Common reference numerals are used throughout the figures to indicate similar features. It should however be noted that even where reference numerals for features used throughout the figures vary, this should not be construed as non-interchangeable or distinct. Indeed, unless specified to the contrary, all features referring to similar components and/or having similar functionalities of all embodiments are interchangeable and/or combinable.

### DETAILED DESCRIPTION

Embodiments of the present invention are described below by way of example only. These examples represent the best ways of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

It should be noted that although exemplary examples, descriptions and/or embodiments are provided under separate headings, these headings should simply serve as a reading aid to provide structure to the description. For the avoidance of any doubt, the features described in any embodiment and/or the embodiments themselves are combinable with the features of any other embodiment and/or any other embodiment unless express statement to the contrary is provided herein. Simply put, the features described herein are not intended to be distinct or exclusive but rather complementary and/or interchangeable.

The present disclosure provides a neural control system using machine learning techniques to analyze neural data to determine, in real time, at least one output neural stimulus required to bring the bodily variable into agreement with, or at least closer to, a desired bodily state, and generating the output neural stimulus, and so provide a closed loop neural control system.

The present disclosure also provides neural control systems providing improved real time performance. The present disclosure also provides updating systems, and auditing and safety systems for neural control systems.

It should be understood that the nervous system of mammals is generally made up of nerves comprising a plurality of neurons and consists of two main parts: the central nervous system (CNS) and the peripheral nervous system (PNS). In most animals and humans, herein referred to as a subject, the CNS includes the brain and the spinal cord, which are made up of special nerves. The PNS includes the somatic nervous system (SoNS) and the autonomic nervous system (ANS), which are made up of many different types of nerves such as, by way of example only but not limited to, afferent nerves (e.g. sensory nerves), efferent nerves (e.g. motor nerves), and/or mixed nerves. The SoNS may carry, by way of example only but is not limited to, conscious motor control for motion and sensation. The ANS may carry, by way of example only but is not limited to, unconscious organ control or unconscious control of bodily functions of the subject.

The SoNS is associated with voluntary control of body movements (e.g. control of skeletal muscles). For example, in the SoNS, afferent nerves include sensory neurons and are responsible for relaying sensation from the body to the CNS and efferent nerves include non-sensory neurons and are responsible for sending out neural information, commands, intent, which may also be referred to as bodily variables as described below, from the CNS to the body (e.g. stimulating muscle contraction). The ANS includes, by way of example only but is not limited to, the sympathetic nervous system (SNS), the parasympathetic nervous system (PSNS) and the enteric nervous system (ENS).

The PNS is essentially a set of nerves that connect the CNS to every other bodily function/body part/portion (e.g. muscles, organs, cells) of the subject. Nerves serve as a conduit for transmission of neural impulses or signals to/from the CNS. For example, SoNS nerves that transmit neural impulses, signals or information from the CNS are called efferent nerves (e.g. motor nerves), while other SoNS nerves that transmit neural impulses, signals or information from one or more parts/portions of the body of the subject to the CNS are called afferent nerves (e.g. sensory nerves). Some nerves in the SoNS may have both efferent and afferent functionality and may be called mixed nerves.

In essence, the nervous system is made up of a set of nerves in which each nerve is made up of a plurality of neurons or a bundle of neurons that receive or transmit such as neural impulses or signals. A neuron has a special cellular structure that allows a nerve to send and propagate neural information rapidly and precisely to other cells, bodily functions or body parts/portions in the body of the subject. For example, the neurons in a nerve include long structures called axons that allow them to send neural impulses or signals in the form of an electrochemical gradient, also known as neural activity. A neuronal population may comprise or represent one or more neurons clustered in a location or a target site on one or more nerves of a subject.

Essentially, neural activity may comprise or represent any electrical, mechanical, chemical and or temporal activity present in the one or more neurons (or the neuronal population), which often make up one or more nerves or section(s) of neural tissue. Neural activity may convey information associated with, by way of example only but not limited to, the body of a subject and/or information about the environment affecting the body of a subject. The information conveyed by neural activity may include data representative of neural data, neural information, neural intent, end effect, tissue state, body state, neural state or state of the body, and/or or any other data, variable or information representative of the information carried or contained in neural activity and interpreted and/or passed by neurons or neuronal populations to the body of the subject. For example, neural data may include any data that is representative of the information or data that is contained or conveyed by neural activity of one or more neurons or a neuronal population. The neural data may include, by way of example only but is not limited to, data representative of estimates of one or more bodily variable(s) associated with the corresponding neural activity, or any other data, variable or information representative of the information carried or contained or conveyed by neural activity.

This information may be represented in an information theoretic point of view as one or more variables associated with the body, which are referred to herein as bodily variable(s). A bodily variable comprises or represents an end effect or tissue state describing a state of some portion of the body, including implanted or wearable medical devices. The bodily variable may itself be classified as a state, sensory, control or other variable based on the role or function of this information and the use of it by the body. Bodily variables can be transmitted to or from the CNS via neural activity in portions of the nervous system. One or more instances of neural activity at one or more neural locations can be said to be an encoding of one or more bodily variables, portions thereof and/or combinations thereof. For example, neural activity of one or more neurons of nerve(s) may be generated or modulated by part of the body to encode one or more bodily variables for reception by other parts of the body, which decode the neural activity to gain access to the bodily variable, portions thereof and/or combinations thereof. Both encoding and decoding of bodily variables can be performed by the CNS and/or bodily tissues therefore facilitating transmission of information around the body of a subject. Bodily variables can be afferent signals transmitted towards the CNS for provision of information regarding the state of bodily variables or efferent signals transmitted away from the CNS for modifying a bodily variable at an end effector organ or tissue.

The values of a group of one or more bodily variables is referred to herein as a bodily state. The bodily state of a subject is the values at a specific time of a collection of one or more relevant bodily variables.

Examples of bodily variables in the organ systems of the body, and often encoded in the ANS, could include parameters such as, by way of example only but is not limited to, current blood glucose concentration, temperature of a portion, part or whole of the body of a subject, concentration of a protein or other key agent, current fullness state of the bladder or bowel, current heart rate or blood pressure, current breathing rate, current blood oxygenation, instructions regarding insulin/glucagon production, instructions regarding heart pacing, instructions regarding blood vessel dilation or constriction for changing blood pressure, instructions regarding changing breathing rate, instructions regarding modifying alveoli dilation to modify oxygen concentration, instructions regarding modifying gastric activity, instructions regarding modifying liver activity, instructions regarding opening/closing of sphincters for voiding/retaining of the bladder or bowel. It is appreciated that bodily variables could be either the raw encodings or combinations of these, for instance bodily variables could include current activity of a whole organ or organ construct or measurements of whole bodily functions or actions such as sweating, defecating, hard breathing, walking, exercising, running etc; each of which it is appreciated could be described as a combination of multiple more fine grained bodily variables. In the ANS, each instance of a bodily variable may be associated with a modified organ function, modifying an organ function, or modifying a bodily function (e.g. one or more bodily variable(s) or the state of an organ or tissue). In other examples, a bodily variable may be associated with any activity in the ANS such as, by way of example only but is not limited to, organ measurement and/or modification of activity.

In another example, in the SoNS, one or more bodily variable(s) generated by the CNS may be transmitted via the PNS as efferent neural activity that is associated with one or more instances of motion (e.g. each bodily variable may be associated with a different motion or movement of a limb, contraction/extension of a single muscle fibre/fibre group/whole muscle/group of muscles, instructions to modify speed/strength length of a muscle contraction, and the like etc.) The CNS may also receive an afferent neural activity encoding a bodily variable corresponding to sensory neural information (e.g. a sensory bodily variable), where in this case the sensory bodily variable represents an encoding of sensory information such as, by way of example only but is not limited to, temperature or pressure on a section or portion of skin, the state of a limb or other muscle group including, angle or position of a joint, position of a whole limb or section of the body, an abstract parameter of activity of the whole body or sub-part of the body, transmitted by one or more neuron(s) or one or more neuronal population(s) associated with the limb or other moving bodily part and the like. The CNS receives the afferent neural activity and then deciphers or decodes this neural activity to understand the sensory bodily variable(s) and responds accordingly.

In another example, a prosthetic limb attached to the body could be represented by a number of bodily variables such as, by way of example only but not limited to, whole prosthetic limb position and orientation, angle or position of prosthetic limb joints, forces or torques exerted by prosthetic limb joints, state of prosthetic limb sensors such as contact force or torque sensors, contact pressure or collision sensors, humidity and temperature sensors. These variables may be encoded into digital or analogue signals and transmitted to the controller by means of electric wires.

Although several examples of bodily variables have been described, this is for simplicity and by way of example only, it is to be appreciated by the skilled person that the present disclosure is not so limited and that there are a plurality of bodily variables that may be generated by the body of a subject and which may be sent between parts of the body or around the body as neural activity. Although neural activity may encode one or more bodily variables, portions thereof and/or combinations thereof, it is to be appreciated by the skilled person that one or more bodily variables of a subject may be measurable, derivable, and/or calculated based on sensor data from sensors capable of detecting and/or making measurements associated with such bodily variables of the subject. It is also to be appreciated by the skilled person that a bodily variable is a direct measurement of any one parameter and could be represented as a generalised parameter of activity or function in an area. This would include bodily variables such as mental states which can not be easily related to low level function such as, experiencing depression, having an epileptic fit, experiencing anxiety, having a migraine.

Although the term bodily variable is described and used herein, this is by way of example only and the present disclosure is not so limited, it is to be appreciated by the skilled person that other equivalent terms from one or more other fields (e.g. medical fields, pharmaceutical fields, biomedical fields, clinicians, biomarker fields, genomics fields, medical engineering fields) may be used in place of the term bodily variable, or used interchangeably or even in conjunction with the term bodily variable, including, by way of example only but is not limited to, one or more of the following terms or fields: vital sign(s), which is often used by clinicians to describe parameters they use for patient monitoring, such as by way of example only but is not limited to, ECG, heart rate, pulse, blood pressure, body temperature, respiratory rate, pain, menstrual cycle, heart rate variation, pulse oximetry, blood glucose, gait speed, etc.; biomarker, which may be used by biologists to describe, by way of example only but is not limited to, protein levels, or measurable indicator of some biological state or condition etc., this term has been further adopted by the Deep Brain Stimulation & Spinal Cord Stimulation clinical fields to refer to recordings of brain wave state or other neural events as well as measurement of environmental conditions including, but not limited to, motion; physiological variable/physiological data, which may often be used by scientists to describe things like ECG, heart rate, blood glucose, and/or blood pressure and the like, this term is also used by Data Sciences International who make implants for recording physiological variables such as ECG, heart-rate, blood pressure, blood glucose, etc.; one or more biosignals, which is often used by medical engineers to describe a signal recording coming from a biological system such as ECoG, ECG, EKG; any information, parameter metric about a subject in, by way of example only but not limited to, the genetic fields including, by way of example only but not limited to, genomic information, epigenetics, phenotype, genotype, other “omics” which can include, by way of example only but is not limited to, transcriptomics, proteomics and metabolomics, microbiomics, and/or other omics related fields and the like; and/or any other term describing a number, metric, state, variable or information associated with the whole body of a subject, any part and/or subpart of the body of the subject and the like.

Although examples of bodily variables are given herein, this is by way of example only and the description is not so limited, it is to be appreciated by the skilled person that the list of bodily variables is extremely large because a bodily variable may be, by way of example only but is not limited to, any number, parameter, metric, variable or information describing some state of the whole body of a subject, any portion, part and/or subpart of the body of the subject and that a bodily variable may be based on, or derived from, one or more combinations of one or more bodily variables or other bodily variables and the like. For example it is appreciated that bodily variables measured at a neurological level, biomarker level, cellular level, and/or tissue level, could combine to form bodily variables observed at a whole system state level such as regarding the vital signs of a subject; physiological meta data of a subject; sensor data representative of one or more bodily variables describing something about the body, parts of the body, or whole body of the subject; state, motion, or output of the body, part of subpart of the body of a subject and the like; modifications thereof, and/or combinations thereof and/or as herein described. Hence it is appreciated that, one or more bodily variables described at one or more higher levels of granularity may be based on a combination of one or more bodily variables described at one or more lower levels of granularity.

Although it is possible to tap into the one or more neuronal population(s) thereby effecting a direct linkage to the nervous system of a subject, there have been problems in capturing and interpreting bodily variable(s) from the neural activity generated by the neuronal population(s) and/or providing or applying neural stimulus signal(s) in order to evoke targeted responses in the form of neural activity in neuronal populations which is equivalent to or directly representing a bodily variable from device(s) to the nervous system of the subject. The bodily variable(s) may be naturally represented by neural activity associated with extremely short electrical pulses from multiple neurons. The neural activity may be received by one or more neural receivers adjacent one or more neurons or neuronal population(s) as neurological signals. These neurological signals may be sampled in which the neurological signal sampling typically provides an information rich dataset that is inordinately large, unwieldy to process, and is usually subject/experiment specific. This has led to attempts at understanding neurological signal(s) by extracting several key features thought to be representative of its information content such as bodily variable(s) encoded as neural activity.

Herein we will refer to samples or ensembles of samples of neural activity as neural biomarkers. A neural biomarker is an objective measurement of a bodily variable, including: biological processes; pathogenic processes; and/or pharmacologic responses to a therapeutic intervention, observed by monitoring one or several neural populations. Wherein neural biomarkers can represent objective indications of medical state. Neural biomarkers can be measured as features, in isolation, or linear or non-linear combinations of features, of the acquired neural population activity, which may be calculated by processing the signals, or learnt by one or more machine learning means, where this learning may be performed by a machine learning processor, either continually or in batch. One or more machine learning models running on a machine learning processor may calculate neural biomarkers having been trained on data from the nervous system of one or more patients, or from a single patient over multiple time periods, or any synthetic or simulated or biological source of neural data or activity. It is appreciated by a professional, skilled in the art, that the learnt neural biomarkers may then be used as time and or subject invariant stationary representations of the activity of the nervous system across a population of patients with the same indication, or for a single patient, or as a representation of the disease when observed in any synthetic or simulated or biological source of neural data or activity. Thus, a neural biomarker represents repeatable features from which the current neural activity can be understood as an indicator of a particular disease state or other physiological state and hence could be used as a basis for treatment decisions, therapeutic design or screening or itself be considered a useful target for direct or indirect modulation by neural, therapeutic or other means.

There is a desire for an efficient mechanism capable of capturing and/or interpreting bodily variable(s) encoded as neural activity and for providing an accurate estimate of one or more bodily variable(s) to a device performing closed loop control associated with one or more bodily functions, one or more body parts and/or portions of the body of a subject. There is a further desire for an efficient mechanism capable of capturing and/or interpreting bodily variable signal(s) produced by a device performing closed loop control associated with one or more body parts or portions of the body of a subject and for providing a corresponding stimulus to the nervous system of the subject associated with the bodily variable signal(s).

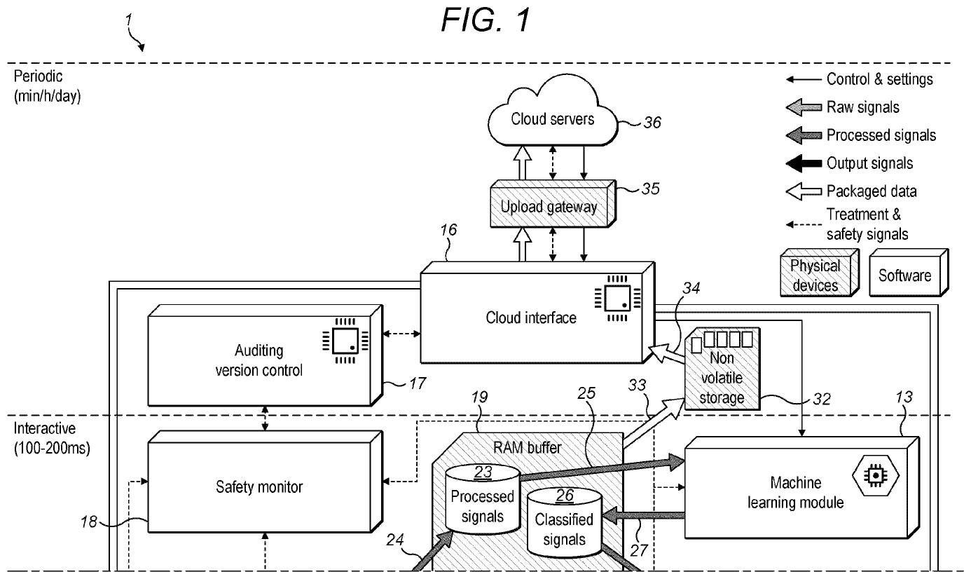

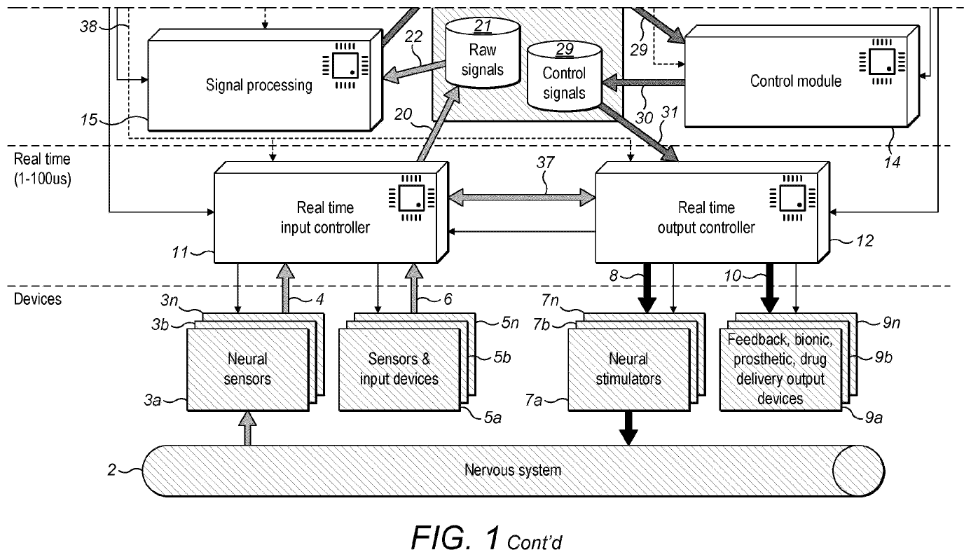

FIG. 1 shows a schematic illustration of the overall software architecture of a neural control system 1 according to an exemplary embodiment.

As shown in FIG. 1, in the illustrated embodiment the neural control system 1 receives neural data from the nervous system 2 of a human or animal subject and subsequently provides neural stimulation to the nervous system 2 of the human or animal subject. It should be understood that although the nervous system 2 is shown as a single item for simplicity the nervous system is made of a large number of nerves, and it is not necessary that the received neural data and neural stimulation relate to the same nerves.

The neural control system 1 receives neural data from a number of neural sensors 3a to 3n implanted in the body of the subject. The neural sensors 3a to 3n are arranged to detect neural signals within the nervous system 2 and convert these into electrical neural data signals 4 which are provided to a real time input controller 11 of the neural control system 1. The real time input controller 11 acts a sensor receiver, receiving neural data from the neural sensors 3a to 3n.

Optionally, the neural control system 1 may also receive data from one or more sensors and/or input devices 5a to 5n. The sensors and/or input devices 5a to 5n are arranged to detect parameter values of bodily variables of the human or animal subject and convert these into electrical data signals 6 which are provided to the real time input controller 11 of the neural control system 1. In some examples some or all of the sensors and/or input devices 5a to 5n may be implanted in the body of the subject. Input devices 5a to 5n may also report a position or state of attached components attached to the body of the subject, such as a prosthetic limb, bionic organ or any other device.

The neural control system 1 also provides neural stimulation control signals to a number of neural stimulators 7a to 7n implanted in the body of the subject. A real time output controller 12 of the neural control system 1 provides electrical neural stimulation control signals 8 to the neural stimulators 7a to 7n. The neural stimulators 7a to 7n are arranged to receive the electrical neural stimulation control signals 8 and apply corresponding neural stimulation signals to the nervous system 2.

The neural stimulators 7a to 7n may comprise any device undertaking an action resulting in or modifying neural activity in a targeted area of the neural tissue of a subject. This could include, by way of example but not limited to, operating modalities such as, electrical stimulation, chemical activation, mechanical stimulation, and/or optogenetic stimulation. It is not necessary that all of the neural stimulators 7a to 7n are the same. In some examples the neural stimulators 7a to 7n may include neural stimulators operating using different modalities being used together.

It should be understood that it is not essential that the neural sensors 3a to 3n are separate devices from the neural stimulators 7a to 7n. Some or all of the neural sensors 3a to 3n and neural stimulators 7a to 7n may be combined in dual purpose sensor/stimulator devices. Hence the illustrated functional separation is not indicative of physical separation.

Optionally, the neural control system 1 may also provide control signals to one or more output devices 9a to 9n, such as feedback devices, bionic organs, drug delivery devices and prosthetic devices, it should be appreciated that this list is by way of example only and that these devices may be either implanted or external to the body of the subject. The neural stimulators 7a to 7n and the output devices 9a to 9n may be collectively referred to as end effectors. The real time output controller 12 of the neural control system 1 provides electrical control signals 10 to the output devices 9a to 9n. The output devices 9a to 9n are arranged to receive the electrical control signals 10 and to carry out corresponding actions in response. In some examples some or all of the output devices 9a to 9n may be implanted in the body of the subject.

The signals may be carried between the neural control system 1, the neural sensors 3, neural stimulators 7, sensors 5 and output devices 9 in any suitable manner. In the illustrated example these signals are carried by a wired communication system.

The neural control system 1 further comprises a machine learning module 13, a control module 14, a signal processing module 15, a cloud interface module 16, an auditing and version control module 17, a safety monitor module 18, and a storage device 19 accessible to the other components. The functions and operation of these different parts of the neural control system 1 will be discussed in more detail below.

In operation of the neural control system 1 the real time input controller 11 receives the neural data signals from the neural sensors 3a to 3n, as indicated by the arrow 4, and optionally receives the data signals from the sensors 5a to 5n, as indicated by the arrow 6. The real time input controller 11 then sends the neural data signals 4, and any data signals 6, to the storage device 19, as indicated by the arrow 20, for storage as raw signal data 21. The raw signal data 21 is so called to indicate that this data has not yet been fully processed by the neural control system 1. However, it is possible that some pre-processing of the neural data signals 4 and the data signals 6 may be carried out by the neural sensors 3a to 3n, sensors 5a to 5n and real time input controller 11 to enable reliable transmission and storing of the data.

The signal processing module 15 obtains raw signal data 21 from the storage device 19, as indicated by the arrow 22, and carries out signal processing of the raw signal data 21 to place the signal data in a suitable format for input to the machine learning module 13. The signal processing module 15 then sends the resulting processed signal data 23 to the storage device 19 for storage, as indicated by the arrow 24. The storage device 19 may be a RAM buffer, for example.

The machine learning module 13 obtains the processed signal data 23 from the storage device 19, as indicated by the arrow 25, and further processes the processed signal data 13 using machine learning models. The machine learning module 13 uses machine learning means, such as one or more machine learning models, to identify, learn, or calculate neural biomarkers of one or more bodily variables in the processed signal data 23 and to determine from these the current status of the bodily variables of the subject. The machine learning module 13 then produces classified signal data 26 defining the determined current status of the bodily variables of the subject and sends the classified signal data 26 to the storage device 19 for storage, as indicated by the arrow 27.

The control module 14 obtains the classified signal data 26 from the storage device 19, as indicated by the arrow 28, and uses machine learning models to determine neural stimulation patterns and modulation required to bring the one or more bodily variables into, or towards, agreement with a desired bodily state of the subject. The control module 14 then produces control signal data 29 defining the required neural stimulation patterns and modulation and sends the control signal data 29 to the storage device 19 for storage, as indicated by the arrow 30. Optionally, the control module 14 may obtain both the classified signal data 26, and also processed signal data 23 relating to bodily variables and derived from data signals from the sensors 5a to 5n, and use machine learning models to determine neural stimulation patterns and modulation required to bring the one or more bodily variables into, or towards, agreement with a desired bodily state of the subject based upon both of these types of data.

The real time output controller 12 obtains the control signal data 29 from the storage device 19, as indicated by the arrow 31, and provides corresponding electrical neural stimulation control signals 8 based on the control signal data 29 to the neural stimulators 7a to 7n. Optionally, the real time output controller 12 may also provide electrical control signals 10 based on the control signal data 29 to the output devices 9a to 9n.

Additionally, the real time input controller 11 sends selected data regarding the neural data signals directly to the real time input controller 11, and the real time output controller 12 sends selected data regarding the electrical neural stimulation control signals directly to the real time input controller 11, as indicated by the arrow 37.

The auditing and version control module 17 ensures that all of the other components of the neural control system 1 are valid, up to date, and operating the correct current versions of any relevant software, firmware, models and/or model parameters. Further, the auditing and version control module 17 schedules, supervises and checks any necessary updating of the components of the neural control system 1, such as updating to new versions of software, firmware, models and/or model parameters. If the auditing and version control module 17 cannot update a component or otherwise ensure a valid version can be operated, the auditing and version control module 17 may signal the safety monitor module 18 described below to bring the system 1 into a safe state and/or disable operation of the whole or part of the system 1. The auditing and version control module 17 may do this if it identifies one or more of incorrect versioning, out of date versioning; and/or unauthorised versioning of components of the neural control system 1.

In some examples the auditing and version module 17 may require periodic authentication of the system 1 with an external authority, such as cloud servers 36 discussed below, and may disable operation or reduce functionality of the whole or part of the system 1 if the authentication of the system 1 has expired or cannot be confirmed within a specified time period.

In some examples the auditing and version module 17 may require periodic updates of the models and/or model parameters. If an update or authentication of a component cannot be successfully performed within the specified time period, the auditing and version control module 17 may disable that component or the whole system 1 or put that component or the whole system 1 into a reduced or basic functionality mode by signaling the safety monitor module 18 described below.

The safety monitor module 18 independently monitors the performance, for example the health, of the components of the neural control system 1, as indicated schematically by the dashed arrows 38. If the safety monitor module 18 identifies an error or failure of one of the components of the neural control system 1 the safety monitor module 18 brings the neural control system 1 into a safe state to ensure safe operation of the neural control system 1. In some examples the safety monitor module 18 may respond to failure by stopping the sending of control signals by the real time output controller 12. In some examples the neural control system 1 may be implemented such that each component and/or subsystem of the neural control system 1 must periodically report its health to the safety monitor module 18, and if any of the health reports indicate a failure, or are incomplete or missed, the safety monitor module 18 will safely shut down the power to critical parts, such as neural transducers, (i.e., neural sensors 3a to 3n and neural stimulators 5a to 5n).

In some examples the safety monitor module 18 may monitor the performance, such as the health, of the components making up the first and/or second control loops discussed herein, and may shut down the power to selected critical parts, or place the system 1 into a safe mode, in response to detecting unacceptable performance, such as a failure, in one of the first and/or second control loops. In particular, the safety monitor module 18 may be arranged to act to reduce or stop the function of any element of the first control loop based on the monitoring of its performance.

In some examples the safety monitor module 18 may monitor the health of the auditing and version module 17 and may shut down the power to selected critical parts, or place the system 1 into a safe mode, or place the system 1 into a reduced or basic functionality mode, in response to detecting a failure to update to a valid version of component or system software, firmware, models and/or model parameters.

In addition to the storage device 19, the neural control system 1 also comprises a non-volatile (NV) data store 32. In operation, the neural control system 1 packages the data stored in the storage device 19 by the various components of the system 1 into a suitable form for transmission and long term storage and supplies this packaged data to the NV data store 32 for storage, as indicated by the arrow 33.

In general, it is expected that the volume of neural data collected and processed by the neural control system 1 will be so great that long term storage of the neural data and related an derived data in the storage device 19 will be impractical. Accordingly, this data is packaged into a suitable form for long term storage in the NV data store 32. The packaged data may be subsequently recovered from the NV data store 32 for review and/or analysis as desired. The packaging the neural data could be any suitable form of compression, metadata or other method of reducing its size while retaining the pertinent information, as appropriate

The cloud interface module 16 periodically obtains packaged data from the NV data store 32, as indicated by the arrow 34, and sends this packaged data through a gateway 35 to cloud servers 36, as indicated by arrow 37, for review and/or analysis. Generally, the cloud interface module 16 will operate as a ‘push’ system, periodically sending new packaged data which has been generated by the neural control system 1 since the last set of packaged data was sent. However, in some examples the cloud interface module 16 may also operate as a ‘pull system, providing new packaged data, or specifically requested parts of the packaged data, on request from the cloud servers 36.

Periodically, or as required, the cloud servers 36 may send updates from the cloud servers 36 via the gateway 35, so that the cloud servers 36 act as an update machine to carry out updating of the neural control system 1. These updates may, for example, be updates to the general firmware of the neural control system 1, the signal processing steps, and the algorithms and models used, including control parameters and settings. This list is exemplary only, and is not intended to be exhaustive. Data may be carried between the neural control system 1 and the cloud servers 36 in any suitable manner. In the illustrated example these signals are carried by an Ethernet wireless communication system.

When the cloud servers 36 act as an update machine for the neural control system 1 the auditing and version module 17 may operate as an auditing module to monitor version control of the neural control system 1. In addition to sending the updates, the cloud servers 36 send information to the auditing and version module 17 regarding the correct version of the neural control system 1, or parts of the neural control system 1. The version of the neural control system is the identities of the updated and/or updateable components of the neural control system 1.

When the auditing and version module 17 has received the information regarding the correct version from the cloud servers 36, the auditing and version module 17 obtains information regarding the current version actually present on the neural control system 1, or parts of the neural control system 1, and compares the current and correct versions. The auditing and version module 17 can instruct the safety module 18 to act to reduce or stop the function of the neural control system 1, or a part of the neural control system 1, based on the correct and current versions, for example if the comparison shows that the current version is not the same as the correct version.

In the illustrated embodiment the cloud servers 36 include systems which retrain machine learning models on high powered cloud servers, and then sends the results of this retraining to the system 1 in the form of updated machine models, or updates to the current machine models. This retraining may be based upon the packaged data from the neural control system 1 and/or on packaged data received from other neural control systems. Periodically, or when updating of the machine learning models is required, updated machine models, or updates to the machine models, can be sent to the neural control system 1 from the cloud servers 36 via the gateway 35 in a similar manner to the updates discussed above. When updated machine learning models or updates to the machine learning models are received by the neural control system 1 the auditing and version control module 17 will carry out and confirm the necessary changes to replace or update the machine learning models currently in use by the neural control system 1.

An example of retraining machine learning models is periodic calibration of the model parameters for a specific patient, which allows for continuous adaptation of a subject specific model to the changes in the subjects nervous system and neural sensors over time, such as neuron degradation and neuron motion relative to the neural sensor. Another example is continuous improvement of the core machine learning model by periodic retraining on the neural data of the whole or a representative subset of a subject population, possibly based on data from multiple different neural control systems.

In the illustrated example, the different modules of the neural control system 1, such as the machine learning module 13, the control module 14, the signal processing module 15, the cloud interface module 16, the auditing and version control module 17, and the safety monitor module 18, and also the real time input controller 11 and the real time output controller 12, are implemented as software modules. In other examples the different modules of the neural control system 1, such as the machine learning module 13, the control module 14, the signal processing module 15, the cloud interface module 16, the auditing and version control module 17, the safety monitor module 18, real time input controller 11 and real time output controller 12, may be implemented as software modules or by dedicated hardware, as convenient in any specific implementation. In some examples different ones of the modules may be implemented in different ways. In particular, in some examples it may be preferred to implement modules carrying out machine learning, such as the machine learning module 13 and/or the control module 14, using dedicated hardware optimized for machine learning processes, such as a GPU or other ASIC optimized for parallel computing.

As is shown schematically in FIG. 1, different parts of the neural control system 1 operate at different response timescales, or response speeds. A first, real time, part of the neural control system 1 operates at real time, or near real time response speeds. The response speed is the time taken between the system 1 receiving an input and generating an output. In the illustrated example this real time part of the neural control system 1 comprises the real time input controller 11 and the real time output controller 12, the connections between them, and their connections to the neural sensors 3a to 3n, sensors and input devices 5a to 5n, neural stimulators 7a to 7n, and output devices 9a to 9n. A second, interactive, part of the neural control system 1 operates at a lower response speed. In the illustrated example this interactive part of the neural control system 1 comprises the machine learning module 13, the control module 14, the signal processing module 15, and the safety monitor module 18, and the connections between them. A third, periodic, part of the system operates at a still lower response speed. In the illustrated example this periodic part of the neural control system 1 comprises the cloud interface module 16, the auditing and version control module 17, and the NV data store 32. In some examples the systems operating in the cloud servers 36 to generate updates to the machine learning models may also be regarded as a part of the periodic part because the updates are provided to and used by the neural control system 1, although the cloud servers 36 are not, strictly, a part of the neural control system 1.

In some examples, the real time part of the neural control system 1 may provide a response speed similar to the response time of a subjects nervous system, while the interactive part of the neural control system 1 may provide a response time similar to the response time of a subjects biological systems controlled by the nervous system, and the periodic part of the neural control system 1 may provide a response speed slower than typical biological systems.

In the illustrated example of FIG. 1 the real time part of the neural control system 1 may have a response time, or response latency, in the range 1 to 100 microseconds (less than 0.001 second), the interactive part of the neural control system 1 may have a response time, or response latency, in the range 100 to 200 milliseconds (0.1 to 0.2 second), and the periodic part of the neural control system 1 may have a response time, or response latency, in the order of minutes, hours or days. In a preferred example the real time part of the neural control system 1 has a response time, or response latency, of less than 100 microseconds. These time ranges are preferred, but are not essential. In other examples, different time ranges may be used. In practice, the response times selected for the system may depend upon the purpose of the system 1, such as the nature of the sensors and devices connected to the system 1.

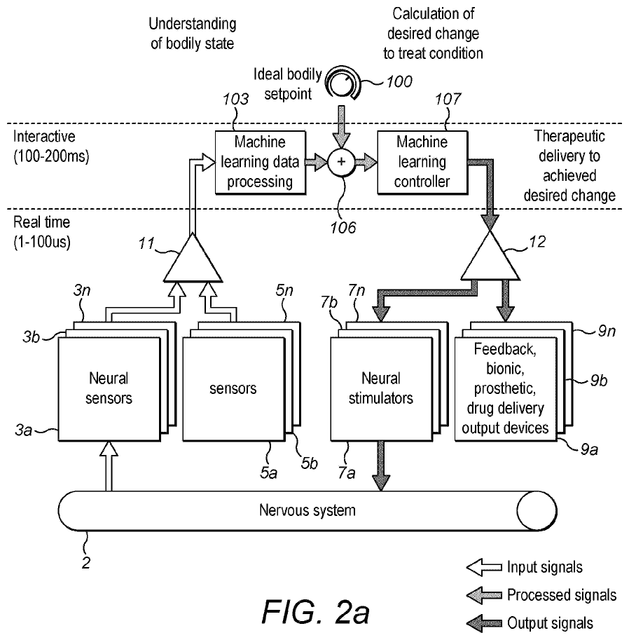

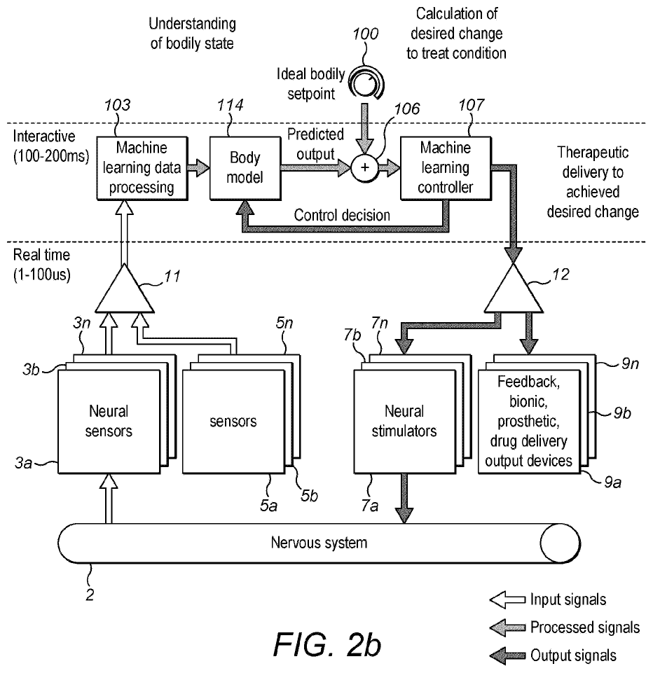

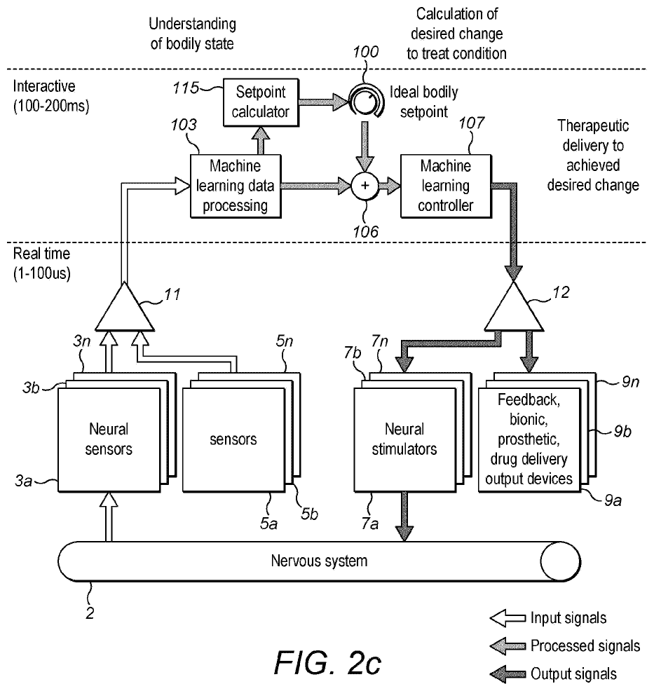

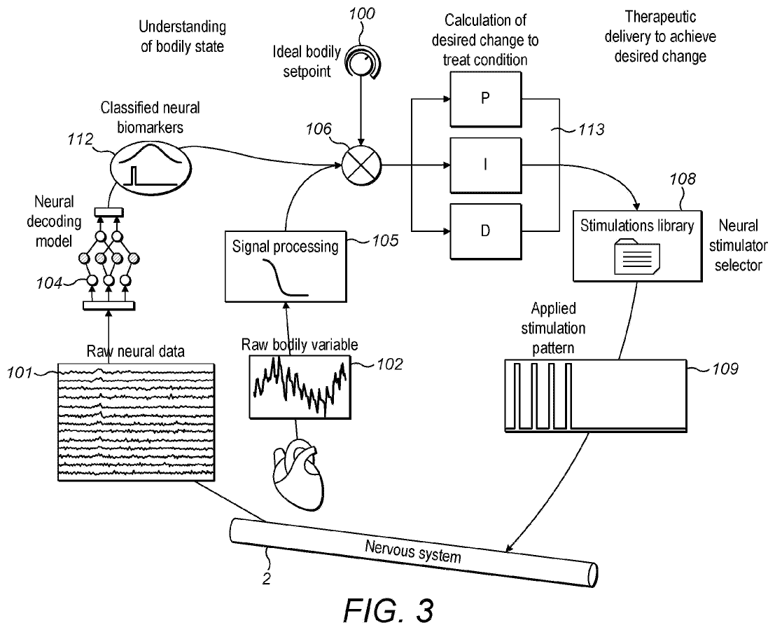

FIG. 2a shows in diagrammatic form a first example of operation of the neural control system 1 of FIG. 1 to provide control of a bodily state of a subject. For clarity only selected parts of the neural control system 1 are shown in FIG. 2a. FIG. 3 shows a representation of a specific example of the data flow through the neural control system 1 in the example of FIG. 2a. The bodily state of a subject is the values at a specific time of a collection of one or more bodily variables. the real time part of the neural control system 1 may, by way of example, have a response time, or response latency, in the range 1 to 100 microseconds.

In the examples of FIGS. 2a and 3 the neural control system 1 is used to provide closed loop control of a bodily state of a subject in order to bring the bodily state into, or at least closer to, agreement with a desired value of that bodily state. In order to do this an ideal bodily setpoint 100 corresponding to a desired value of the bodily state being controlled is set. This ideal bodily setpoint 100 may be fixed, or may be determined based on information about the subject. The ideal bodily setpoint 100 may be pre-set, may be determined by the neural control system 1, or may be provided to the neural control system 1 by another system, as appropriate in any specific implementation. By a bodily setpoint is meant the standard meaning in the technical field of the invention of a setpoint for a control system, and is the desired or target value for bodily variable or bodily state. Departure of the bodily variable or bodily state from its setpoint is one basis for error-controlled regulation using negative feedback for automatic control.

In the specific example of FIG. 3 the bodily state is the heart rate of the subject, and the ideal bodily set point 100 is a specific heart rate value. In some examples a desired value for the heart rate may be calculated by the neural control system 1 based, for example, on other bodily variables, e.g., the current blood pressure or current physical activity level or overall cardiovascular health of the subject. In other examples the desired value for the setpoint of heart rate may be provided to the neural control system 1 by a clinician.

In operation of the neural control system 1 in the first example of FIG. 2a, the neural sensors 3a to 3n obtain neural data relating to a bodily state of the subject from the subjects nervous system 2, and the sensors 5a to 5n obtain bodily variable data regarding bodily variable parameter values. FIG. 3 shows illustrative examples of full spectrum multiple channel neural data 101 relating to a heart rate of the subject which may be produced by the neural sensors 3a to 3n, and blood pressure values 102 which may be produced by sensors 5a to 5n.

In the first example, the neural data and body variable data is provided by the neural sensors 3a to 3n and the sensors 5a to 5n to the real time input controller 11 in the real time part of the neural control system 1, which sends this data to the interactive part of the neural control system 1 for processing. In the interactive part of the neural control system 1 the neural data and body variable data is subject by a machine learning (ML) data processing 103, comprising processing by the signal processing module 15 to produce processed signal data 23, and processing using an ML model of at least the neural data by the machine learning module 13 to produce classified signal data 26. As shown in FIG. 3, classified neural biomarkers 112 may be produced by processing of the full spectrum multiple channel neural data 101 using a neural decoding machine learning model 104 by the machine learning module 13. The example of FIG. 3 further shows signal processing 105 applied to the blood pressure values 102 by the signal processing module 15.

In the first example, the output data produced by the data processing 103 is compared at a summing junction 106 with the ideal bodily set point 100 to calculate a desired change in the value of the bodily state to bring the bodily state into, or at least closer to, agreement with the ideal bodily set point. The summing junction 106 compares the inputs it receives of the bodily state values of the output data and the values of the ideal bodily setpoint 100, and outputs the difference between these values. may be carried out by the control module 14. As shown in FIG. 3, the output of the summing junction 106 may be used in a PID (proportional-integral-derivative) calculation by a PID controller 113, as commonly used in feedback control systems. PID controllers are well known to the skilled person, so it is not necessary to describe this in detail herein. In the illustrated first example the summing junction 106 carries out a simple subtraction of its two inputs. In other examples different comparisons, such as more complex operations, may be carried out.

In the first example, the desired change in the value of the bodily state produced by the summing junction 106 is subjected to data processing 107 using an ML model by the control module 14 to determine a set of neural stimulation signals required to bring about the desired change. As shown in FIG. 3 the ML model may be a simulation library selector 108 selecting an appropriate set of neural stimulation signals from a library of possible stimulations. The set of neural stimulation signals may, for example, define which of the neural stimulators 7a to 7n are to produce neural stimulation signals, and the timing and modulation of these neural stimulation signals. FIG. 3 shows an illustrative example 109 of a pattern of applied stimulation signals comprising a set of neural stimulation signals.

In the first example, the determined set of neural stimulation signals is provided by the interactive part of the neural control system 1 to the real time output controller 12 in the real time part of the neural control system 1, which determines and sends corresponding electrical neural stimulation control signals to selected ones of the neural stimulators 7a to 7n as required for the neural stimulators 7a to 7n to generate and apply the desired set of neural stimulation signals to the subject nervous system 2.

FIG. 2b shows in diagrammatic form a second example of operation of the neural control system 1 of FIG. 1 to provide control of a bodily state of a subject. For clarity, only selected parts of the neural control system 1 are shown in FIG. 2b. The second example of FIG. 2b is generally similar to the first example of FIG. 2a, and only the differences will be discussed in detail.

In the second example of FIG. 2b the neural control system 1 utilizes a body model 114. The body model 114 is an internal dynamical model of the body of the subject, which is used by the neural control system 1 to calculate the optimum actions for control. The body model may, for example, be a white box model, an input/output model, a state space model, or any other model of the system.

In the second example the output data produced by the data processing 103 is input to the body model 114 and used to produce an estimate or prediction of a current bodily state of a subject using a model predictive control process (MPC) process. Accordingly, the estimated or predicted current state of the body is informed by a combination of the neural biomarkers produced by the ML data processing 103 and/or the data from sensors 5a-n.

The current bodily state is output from the body model 114 and compared at a summing junction 106 to an ideal bodily set point 100. A machine learning controller 107 performs optimization of the receding control horizon to calculate optimal control actions to cause a desired change in the value of the bodily state to bring the bodily state into, or at least closer to, agreement with the ideal bodily set point, and output the desired change to determine a set of neural stimulation signals required to bring about the desired change, in a similar manner to the first example described above. In some examples information regarding the determined set of neural stimulation signals is reported to the body model 114 by the machine learning controller 107 and used by the body model 114 to update the body model 114 to take into account the expected effect of the determined set of neural stimulation signals. This second example is an example of a Model Predictive Controller. Model Predictive Controllers are well known to the skilled person, so it is not necessary to describe this in detail herein.

In one embodiment of the second example the body model 114 uses the history of past control moves and the subsequent changes in bodily variables to update the body model.

FIG. 2c shows in diagrammatic form a third example of operation of the neural control system 1 of FIG. 1 to provide control of a bodily state of a subject. For clarity, only selected parts of the neural control system 1 are shown in FIG. 2c. The third example of FIG. 2c is generally similar to the first example of FIG. 2a, and only the differences will be discussed in detail.

In the third example of FIG. 2c the neural control system 1 utilizes a setpoint calculator 115. The ML data processing 103 provides the bodily variable data and/or neural signal data it has received to the setpoint calculator 113. The setpoint calculator 113 then uses the received bodily variable data and/or neural signal data to calculate an estimate of the ideal bodily setpoint 100 and outputs this estimated ideal bodily setpoint 100 to the summing junction 106. machine learning controller 107. The setpoint calculator 115 may alternatively receive bodily variables from the input controller 11, or directly from the sensors 3a-n and 5a-n, or via any other means.

In other examples not using a summing junction the setpoint calculator 115 may provide the calculated ideal bodily setpoint 100 to the data processing 107 using an ML model.

In examples similar to the specific example of FIG. 3 where the bodily state is the heart rate of the subject, the setpoint calculator may, for example, choose to reduce the bodily setpoint of heart rate when a bodily variable of blood pressure increases to dangerous levels, or is increasing dangerously rapidly,

The system architecture and method of operation of the neural control system 1 in the first to third examples described above enables the neural control system 1 to provide a control loop allowing effective closed loop control based upon real time nerve information. Further, the neural control system 1 can provide closed loop performance based directly on organ performance and/or biologically or medically relevant features derived from the received neural signals.

The description of the first to third examples set out above discusses examples where a set of neural stimulation control signals are output to neural stimulators 7a to 7n based on a input neural data from neural sensors 3a to 3n and body variable data from sensors 5a to 5n. It should be understood that these are only some illustrative examples.

In other examples, the output may comprise control signals to one or more of the output devices 9a to 9n in addition to, or instead of, neural stimulation control signals to neural stimulators 7a to 7n. Further, the input may comprise only neural input data from neural sensors 3a to 3n or only body variable data from sensors 5a to 5n.

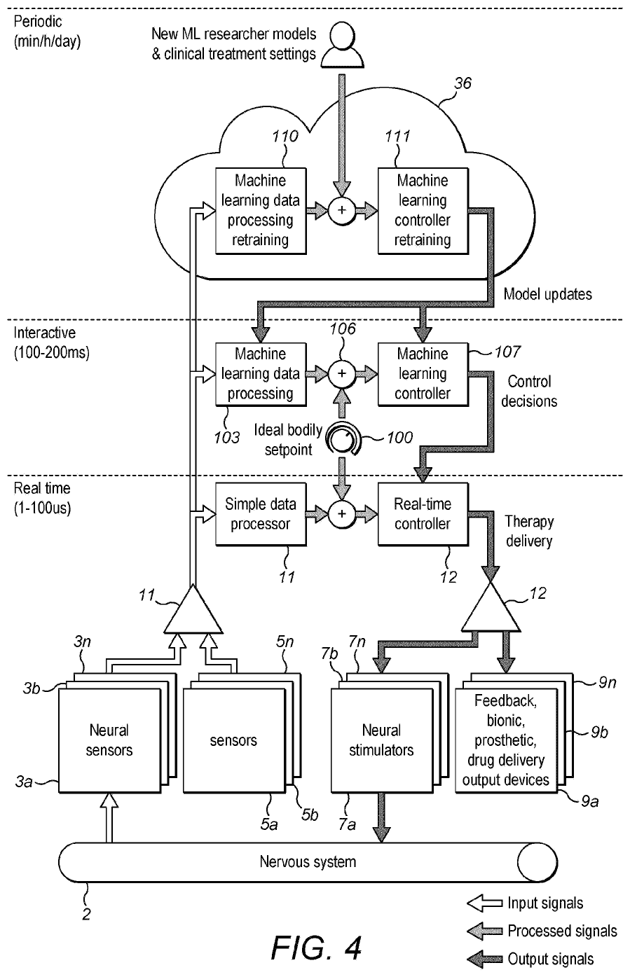

FIG. 4 shows in diagrammatic form a fourth example of operation of the neural control system 1 of FIG. 1 to provide control of a bodily state of a subject. For clarity only selected parts of the neural control system 1 are shown in FIG. 4.

In FIG. 4 the neural control system 1 is used to provide closed loop control of a bodily state of a subject in order to bring the bodily state into, or at least closer to, agreement with a desired value of that bodily state. In order to do this an ideal bodily setpoint 100 corresponding to a desired value of the bodily state being controlled is set. Similarly to the examples of FIGS. 2a to 2c, the ideal bodily setpoint may be fixed, or may be determined based on information about the subject. The ideal bodily setpoint may be pre-set, may be determined by the neural control system 1, for example by a setpoint calculator 115, or may be provided to the neural control system 1 by another system, as appropriate in any specific implementation. In some examples the ideal bodily setpoint may be set in advance as a treatment to bring the bodily state to a desired value of the bodily state.

In examples where the ideal bodily setpoint is determined by the neural control system 1 the ideal bodily setpoint may be calculated based on the received data from the neural sensors 3a to 3n and/or the sensors and/or input devices 5a to 5n. The ideal bodily setpoint may be calculated by a machine learning (ML) model.

In the fourth example illustrated in FIG. 4 the neural control system 1 forms a primary control loop providing closed loop control of a bodily state of a subject, this primary control loop corresponding to the control loop provided in the first example of FIG. 2a. This primary control loop is a first control loop provided by the neural control system 1.

In operation of the neural control system 1 in the illustrated example of FIG. 4, similarly to the first example of FIG. 2a, the neural sensors 3a to 3n obtain neural data relating to a bodily variable of the subject from the subjects nervous system 2, and/or the sensors 5a to 5n obtain bodily variable data regarding bodily variable parameter values.

In the fourth example, similarly to the first example, the neural data and/or body variable data is provided by the neural sensors 3a to 3n and the sensors 5a to 5 to the real time input controller 11 in the real time part of the neural control system 1, which sends this data to the interactive part of the neural control system 1 for processing. In the interactive part of the neural control system 1 the neural data and body variable data is subject to data processing 103, comprising processing by the signal processing module 15 to produce processed signal data 23, and processing using an ML model of at least the neural data by the machine learning module 13 to produce classified signal data 26. The output data produced by the data processing 103 is compared at a summing junction 106 with an ideal bodily set point 100 to calculate a desired change in the value of the bodily state to bring the bodily state into, or at least closer to, agreement with the ideal bodily set point 100. The functionality of the summing junction 106 may be carried out by the control module 14 in some examples. The degree to which the value of the bodily state is different from the setpoint value, as determined by the summing junction 106, is subjected to data processing 107 using an ML model by the control module 14 to determine a set of neural stimulation signals and/or required to bring about the desired change. The set of neural stimulation signals may, for example, define which of the neural stimulators 7a to 7n are to produce neural stimulation signals, and the timing and modulation of these neural stimulation signals, and/or what control signals are to be sent to output devices 9a to 9n.

In the fourth example, similarly to the first example, the determined set of neural stimulation signals and/or control signals is provided by the interactive part of the neural control system 1 to the real time output controller 12 in the real time part of the neural control system 1, which determines and sends corresponding electrical neural stimulation control signals to selected ones of the neural stimulators 7a to 7n and/or corresponding control signals to selected ones of the output devices 9a to 9n, as required for the neural stimulators 7a to 7n and/or output devices 9a to 9n to generate and apply the desired set of neural stimulation signals to the subject nervous system 2 and/or actions to be taken by the output devices 9a to 9n.

Accordingly, the fourth example of FIG. 4 provides a primary control loop through the interactive part of the neural control system 1 corresponding to the control loop of the first example of FIG. 2a.

The fourth example of FIG. 4 further sends selected data regarding the neural data signals directly from the real time input controller 11 to the real time output controller 12. The selection of the selected data regarding the neural data may be carried out by a data processing function of the real time input controller 11. In general, this data processing function will be relatively simple compared to the ML processing carried out in the intermediate part of the neural control system 1.

The selected data regarding the neural data signals sent directly from the real time input controller 11 to the real time output controller 12 identifies the timing of the sensed neural data signals. In other examples, this data may additionally or alternatively identify other features of the neural data signal such their frequency, or other simple signal features easily calculable in the short response or operating time of the real time controller 11.

The determined set of neural stimulation signals provided by the interactive part of the neural control system 1 to the real time output controller 12 in the real time part of the neural control system 1 may specify a desired timing of the neural stimulation signals relative to the natural neural signals carried by the subjects nervous system 2. This desired timing may specify how the neural stimulation signals are to be synchronized with the natural neural signals. For example, if the neural stimulation signals are intended to provide an effect in a biological system such as the cardiac system which has an oscillatory function it can be beneficial to provide neural stimulation signals during a specific phase of the heart beat cycle, or the corresponding oscillatory neural control of the heart beat cycle.

Accordingly, the real time output controller 12 uses the timing of the sensed neural data signals indicated by the selected data received from the real time input controller 11 as at least a part of the basis for controlling the timing of the electrical neural stimulation control signals to selected ones of the neural stimulators 7a to 7n, so that the neural stimulators 7a to 7n generate and apply the desired set of neural stimulation signals to the subject nervous system 2 at the specified desired timing relative to the natural neural signals. The specified timing may be any desired delay, including a zero delay or simultaneity, or as close to simultaneity as can be achieved.

It will be understood that identifying the timing of suitable natural neural signals may be carried out using a simple data processing function which is quicker and simpler than the ML processing carried out in the intermediate part of the neural control system 1.

The direct transfer of selected data regarding the neural data signals between the real time input controller 11 and the real time output controller 12 within the real time part of the neural control system provides an additional high speed, or low delay/latency, control loop for controlling the timing of neural stimulation relative to the natural neural signals. This high speed control loop allows the timing of the neural stimulation relative to the natural neural signals to be controlled more accurately than can be achieved by the primary control loop, which is delayed by the time required to carry out the more complex signal processing, particularly the ML processing.

The selected data regarding the neural data signals sent directly from the real time input controller 11 to the real time output controller 12 may also identify the amplitude of the sensed neural data signals and/or sensor signals.

Further, the interactive part of the machine learning system 1 could set a desired control output, for example a desired amplitude and/or duration of stimulation to the nervous system or a desired amplitude and/or duration of operation of an end effector device, such as movement of a prosthetic device, and specify this desired control output to the real time output controller 12.

Accordingly, following the application of neural stimulation signals to the subject nervous system 2, or the application of control signals to the output devices 9a to 9n, the real time output controller 12 monitors the amplitude of the sensed neural data signals and/or data signals indicated by the selected data received from the real time input controller 11. If the indicated amplitude of the sensed neural data signals indicates a response which is not in agreement with the desired amplitude and/or duration of stimulation or operation, the real time output controller 12 responds by controlling the modulation of subsequent neural stimulation signals or control signals to change their amplitude and/or frequency as appropriate to bring the response which into, or towards, agreement with the desired amplitude and/or duration of stimulation or operation. In some examples the cycle of sensing and amplitude modulation may be repeated until the amplitude of the sensed response is in agreement with the desired level. The real time output controller 12 may, for example operate as a proportional response controller or a PID controller.

Accordingly, the modulation of neural stimulation signals or control signals, that is, their amplitude and/or frequency, or other defining parameter may be based at least partially on the amplitude, frequency, timing or other characteristic feature of sensed neural data signals and/or data signals. The sensed neural data signals may be neural data signals having any source. In particular, the sensed neural data signals may be natural neural data signals or evoked neural data signals that are the result of applied neural stimulation or other action by an end effector device, such as an end effector device acting on the body of the subject.

In some examples the response may be compared to a maximum safe amplitude threshold, and if the response is above this threshold the modulation of subsequent neural stimulation signals or control signals is changed to alter their amplitude and/or frequency until the response is reduced to a level below the maximum safe amplitude threshold. In some examples, if the response is above the maximum safe amplitude threshold the application of subsequent neural stimulation signals and/or control signals may be stopped, for safety.

It will be understood that identifying the amplitude of a response from suitable neural signals and data signals may be carried out using a simple data processing function which is quicker and simpler than the ML processing carried out in the intermediate part of the neural control system 1.

For example, if the output devices 9a to 9n control a prosthetic limb device and at least one of the sensors and/or input devices 5a to 5n is an IMU sensor providing information about the current movement speed of the prosthetic limb device, the control module 14 may provide a set of control movements to be executed by the prosthetic limb device and controlled by a set of control signals for the output devices 9a to 9n, together with a desired movement speed for the prosthetic limb device. The information about the current movement speed of the prosthetic limb device provided to the real time output controller 12 by the real time input controller 11 can be used by the real time output controller 12 to modulate the subsequent control signals sent to the output devices 9a to 9n in order to move the prosthetic limb device at the desired speed. It should be understood that any of the output devices 9a to 9n could physically be the same device connected over the same connection as any input devices 5a to 5n, for instance a motor being controlled as an output device may report its angular position as an input device. Hence functional separation is not indicative of physical separation.

In another example, at least one of the sensors 5a to 5n may be a blood pressure sensor sensing blood pressure of the subject, and the neural stimulators 7a to 7n may be arranged to stimulate the nervous system 2 to increase blood pressure of the subject. The control module may provide a set of neural stimulation signals to increase blood pressure together with a desired blood pressure value. The information about the current blood pressure provided to the real time output controller 12 by the real time input controller 11 can be used by the real time output controller 12 to modulate the neural stimulation control signals sent to the neural stimulators 7a to 7n in order to maintain the blood pressure at the desired value. For example, if the sensor signals indicated that blood pressure was dropping below the desired value the real time output controller 12 can modulate the neural stimulation control signals to increase the amplitude of the applied neural stimulation. This mode of action is not limited to use in relation to blood pressure, and may be desirable in any system where the response is required to be modulated within the timescales of the real time controller, for either safety or functional reasons, before the next control action is decided by the primary control loop.

The direct transfer of selected data regarding the neural data signals and/or data signals between the real time input controller 11 and the real time output controller 12 within the real time part of the neural control system provides an additional high speed, or low delay/latency, control loop for controlling and/or modulating the amplitude of neural stimulation and/or other control outputs. The controlled and/or modulated parameters could include, by way of example but not limited to, the frequency, duration, amplitude or onset of the neural stimulation signal emitted by the neural stimulators. This high speed control loop allows the timing of the neural stimulation relative to the natural neural signals to be controlled and modulated more rapidly than can be achieved by the primary control loop, which is delayed by the time required to carry out the more complex signal processing, particularly the ML processing. This high speed control loop is a second control loop provided by the neural control system 1, and has a lower response latency than the first control loop.

Further, critical threshold values for parameters measured by some of the sensors 5a to 5n, and corresponding remedial actions to be taken, may be set in the real time output controller 12. The real time output controller 12 monitors the amplitude of the sensed data signals corresponding to these parameters indicated by the selected data received from the real time input controller 11, and in response to the parameter value exceeding the threshold value, by sends control signals and/or neural control signals to the output devices 9a to 9n and the neural stimulators 7a to 7n corresponding to the remedial action.

For example, one or more of the sensors 5a to 5n may be temperature sensors measuring the temperature at a surface or extremity of a prosthetic limb device, and the corresponding remedial action may be a rapid movement of the prosthetic limb device in a direction which withdraws that surface or extremity. When the sensors 5a to 5n indicate that the sensed temperature exceeds a critical threshold value the real time output controller 12 sends a predetermined set of control signals and/or neural control signals which will cause the output devices 9a to 9n and the neural stimulators 7a to 7n to rapidly withdraw the prosthetic limb device.

This may provide an ‘artificial spinal reflex’ to protect the subject, or the subjects prosthetic devices, from accidental harm.

The direct transfer of selected data regarding the neural data signals and/or data signals between the real time input controller 11 and the real time output controller 12 within the real time part of the neural control system provides an additional high speed, or low delay/latency, control loop for controlling immediate responses to the sensing of dangerous conditions. This high speed control loop allows the dangerous conditions to be responded to more quickly than can be achieved by the primary control loop, which is delayed by the time required to carry out the more complex signal processing, particularly the ML processing.

In some examples the high speed control loop formed by the direct transfer of selected data regarding the neural data signals and/or data signals between the real time input controller 11 and the real time output controller 12 in the real time part of the neural control system 1 may be used as the main control loop to carry out control of a bodily variable, with the primary control loop formed by the interactive part of the neural control system 1 being activated or used more sparsely, for only part of the time, in order to reduce power demands of the neural control system 1. In some such examples the primary control loop formed by the interactive part of the neural control system 1 may be activated periodically, or may be activated based upon the values of the neural data signals and/or data signals. For example, the primary control loop formed by the interactive part of the neural control system 1 may be activated only when the values of one or more selected neural data signals and/or data signals cross predetermined thresholds or limits. This may be particularly useful to minimize power consumption in applications where the neural control system 1 is implanted in the subject body.

Further, a determined set of neural stimulation signals provided by the interactive part of the neural control system 1 to the real time output controller 12 in the real time part of the neural control system 1 may specify a maximum safe amplitude of natural neural signals generated in response to the neural stimulation signals. For example, should a stimulation amplitude be calculated by the interactive control loop to be 1 mA, and evoke a natural neural signal voltage of greater than 3000 μV, where 3000 μV was the maximum safe amplitude, the amplitude of the stimulation signal would be arrested or reduced

Accordingly, following the application of neural stimulation signals to the subject nervous system 2 the real time output controller 12 monitors the amplitude of the sensed neural data signals indicated by the selected data received from the real time input controller 11. If the indicated amplitude of the sensed neural data signals exceeds the maximum safe amplitude the real time output controller 12 responds by controlling the modulation of subsequent neural stimulation signals to reduce their amplitude until the indicated amplitude of the sensed neural data signals is reduced to a safe level below the maximum safe amplitude. In some examples the cycle of sensing and amplitude reduction may be repeated until the amplitude of the sensed neural data signals is reduced to a safe level. In some examples the application of subsequent neural stimulation signals may be stopped, corresponding to a reduction in amplitude to zero, for safety.

It will be understood that identifying the amplitude of suitable natural neural signals may be carried out using a simple data processing function which is quicker and simpler than the ML processing carried out in the intermediate part of the neural control system 1.

The direct transfer of selected data regarding the neural data signals between the real time input controller 11 and the real time output controller 12 within the real time part of the neural control system provides an additional high speed, or low delay/latency, control loop for modulating and/or controlling the amplitude of neural stimulation signals based on the amplitude of the sensed neural signals generated in response to previous neural stimulation signals. This high speed control loop allows an unsafe level of neural response to the neural stimulation to be identified and responded to and corrected more quickly than can be achieved by the primary control loop, which is delayed by the time required to carry out the more complex signal processing, particularly the ML processing. This quicker correction may allow harm which could potentially otherwise be caused to be prevented or mitigated.

As is explained above regarding the fourth example of FIG. 4, the direct transfer of selected data regarding the neural data signals between the real time input controller 11 and the real time output controller 12 within the real time part of the neural control system can provide one or more high speed, or low delay/latency, control loops in addition to the primary control loop according to the first to third examples of FIGS. 2a to 2c.

The fourth example of FIG. 4 further sends selected data regarding the timing of neural stimulation signals directly from the real time output controller 12 to the real time input controller 11.

In examples where the locations of the neural sensors 3a to 3n and the neural stimulators 7a to 7n on the subject nervous system are such that at least some of the neural sensors 3a to 3n may directly receive the neural stimulation signals generated by the neural stimulators 7a to 7n, the real time input controller 11 can respond to the selected data regarding the timing of neural stimulation signals received from the real time output controller 12 by stopping the recording of neural data by the affected ones of the neural sensors 3a to 3n for the duration of the neural stimulation signals, in order to prevent cross talk between the neural stimulators 7a to 7n and the neural sensors 3a to 3n reducing the quality of the received neural data. In some examples the affected neural data may be replaced by blanket zeros during the stimulation. In some examples the neural sensors 3a to 3n may be switched off or deactivated for the duration of the neural stimulation signals.

In the examples of FIGS. 2 to 4 the neural controller 1 provides a single output of nerve stimulation signals to control a single bodily variable. In other examples the neural controller 1 may provide multiple sets of nerve stimulation signals to control multiple bodily variables, and/or multiple sets of outputs thorough multiple output devices.

In the fourth example of FIG. 4, the neural control system 1 sends data to cloud servers 36. In the fourth example of FIG. 4 the cloud servers 36 operate as an update machine. In order to enable operation as an update machine the cloud servers 36 include machine learning data processing retraining systems 110 and machine learning controller retraining systems 111. The machine learning data processing retraining systems 110 and machine learning controller retraining systems 111 retrain machine learning models using high powered computers, such as cloud computers, based on the data provided by the neural controller 1. This retraining may be carried out with input from machine learning researchers developing new and improved machine learning models. This retraining may also be based on data received from other neural controllers additional to the neural control system 1.

The cloud servers 36 generate updated machine learning models by machine learning training using the neural data received from the neural control system 1, and possibly also other data from other sources. The cloud servers 36 may periodically, or as necessary, send updated machine learning models or machine learning model updates to the neural control system 1. The neural control system 1 receives the updated machine learning models or machine learning model updates from the cloud servers 36, or other update machines, and uses these to update or replace the machine learning model or models used by the machine learning module 13 and the control module 14, as required.

The updates to the machine learning models may be calculated based on the received neural data, calculated neural biomarkers, output signals, recorded neural data, data from other sensors, and/or data representing bodily state and/or any other data saved by the neural control system. The updates may be generated based on data recorded by the neural control system 1 during specific periods of guided activity during rehabilitation or recalibration periods.

In examples where the cloud servers 36 act as an update machine the update machine may be provided by an automated cloud system.

In some examples the update machine may be a manual connection over a local wired or wireless connection. In some examples the updates may be automatically calculated by one or more machine learning systems for calculating long term treatment. In some examples the updates may be chosen by a treating clinician.

The system architecture and method of operation of the neural control system 1, and its interaction with the cloud servers 36 described above enables the neural control system 1 to be provided with an additional control loop allowing updating of the machine models used based upon data obtained from the subject and the performance of the neural control system 1. This additional control loop is relatively low speed, or relatively high delay/latency compared to the primary control loop. The additional control loop enables performance of the machine learning models, and thus the neural control system 1, to be improved over time as more data is gathered by the neural control system 1, and other neural control systems

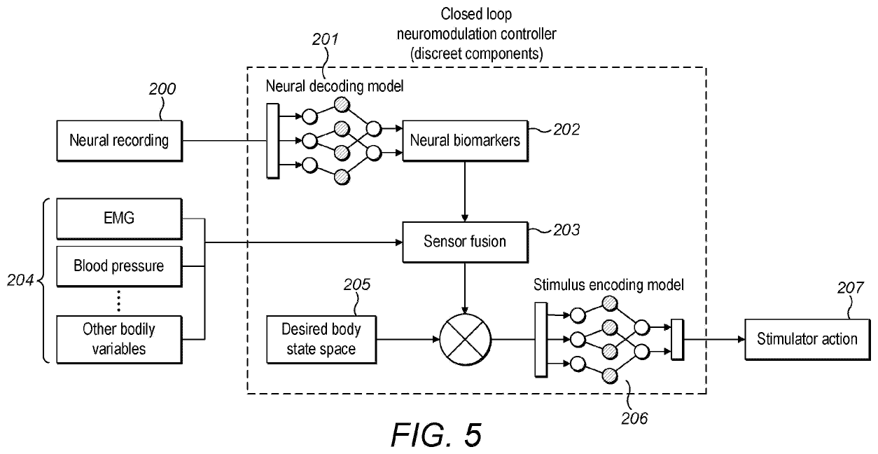

FIG. 5 shows a schematic diagram of an alternative example of a closed loop control system useable in the neural control system 1. In the example of FIG. 5, neural data 200 is subject to processing by a first ML neural decoding model 201 to decode the neural data and identify neural biomarkers 202. This processing by the first ML model 201 may be carried out by the machine learning module 13.

The identified neural biomarkers 202 are then combined in a sensor fusion process 203 with bodily variable data 204. The resulting fused data is then combined with an ideal, or desired, bodily set point 205, to calculate a desired change to bring the current body state into agreement with the ideal bodily set point.

The calculated desired change is then subject to processing by a second ML stimulus encoding model 206 to identify stimulator actions 207, or other actions, required to make the desired change. This processing by the second stimulus encoding ML model 206 may be carried out by the control module 14. In some examples the second stimulus encoding ML model 206 may be carried out by a machine learning output processor.

The identified actions are then output in the form of suitable control signals.

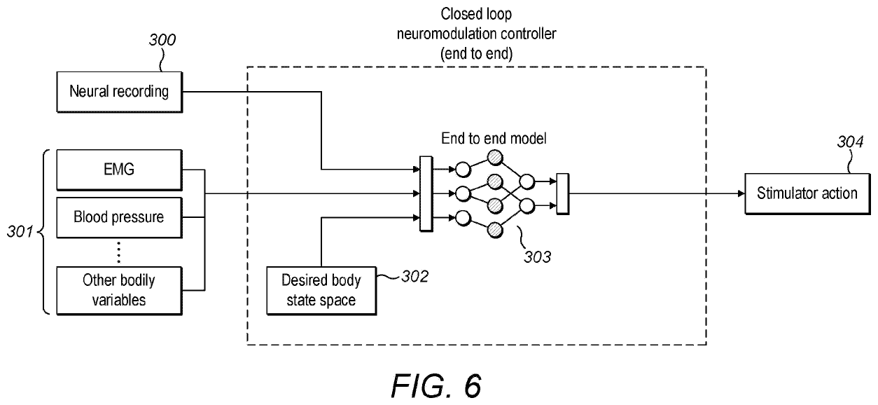

FIG. 6 shows a schematic diagram of another alternative example of a closed loop control system useable in the neural control system 1. In the example of FIG. 6, neural data 300, bodily variable data 301, and an ideal, or desired, bodily set point 302 are all subject to processing by a single ‘end-to-end’ ML model 303 to directly identify stimulator actions 304, or other actions, required to make the desired change. This processing by a single ‘end-to-end’ ML model may be carried out a combined machine learning module replacing both the machine learning module 13 and the control module 14 of FIGS. 1 to 5.

The identified actions are then output in the form of suitable control signals.

In all of the examples described above, the required output actions, for example movement of a prosthetic limb, an action of a bionic organ, or a stimulation of a portion of the subject nervous system to have a desired biological end effect, are derived from the content received from the neural sensors and/or other sensors and/or input devices. The output of a machine learning model, module, processor or controller can be directly used to control end devices, or alternatively can be used to create modifications to a continuous device function. For example, a robotic leg could operate a continuous program of stepping pattern with initiation of walking and speed of walking modulated by the output of the machine learning model. In another example a device stimulating on oscillatory circuit in the body such as the cardiac pulse could have a preprogrammed cycle that is modulated based on the output of the machine learning model.

In some examples the machine learning module and control module operate on separate timing loops with the control module potentially taking one or more outputs from the machine learning module before taking action. Further, either or both of the machine learning module and control module may operate on an asynchronous timescale and only process a section of data and produce output if certain conditions of the data input are met such as, for example, above an absolute value or that one of the sensors reads in a certain range.

It should be appreciated that the machine learning modules and processes set out above are described by way of example only and that any number of machine learning models could be used, utilizing many types of architecture to process data. In some examples multiple machine learning models may be run simultaneously in parallel with a majority vote, state space model or other decision making module deciding on the device output action based on the outputs of multiple ones of the machine learning models. The different machine learning models may be of different types and operate over different timescales.

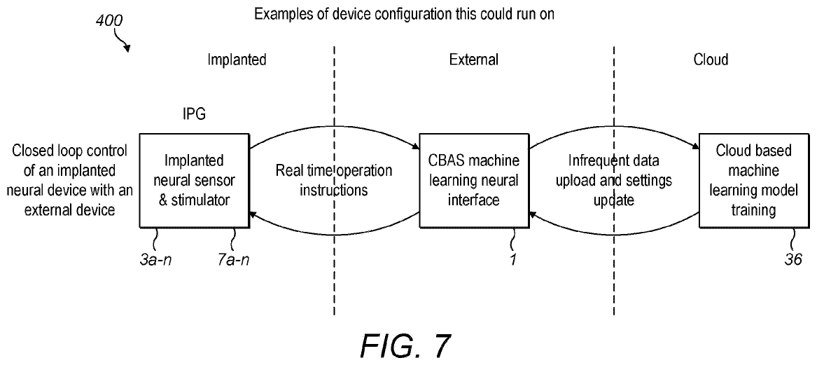

FIG. 7 shows a first example of a configuration of a system 400 incorporating the neural control system 1 to control an implanted neural device. In the first example the neural sensors 3a to 3n and stimulators 7a to 7n are implanted in the body of the subject, the neural control system 1 is located externally of the subject, and the cloud servers 36 are located remotely from the neural control system 1 and the subject.

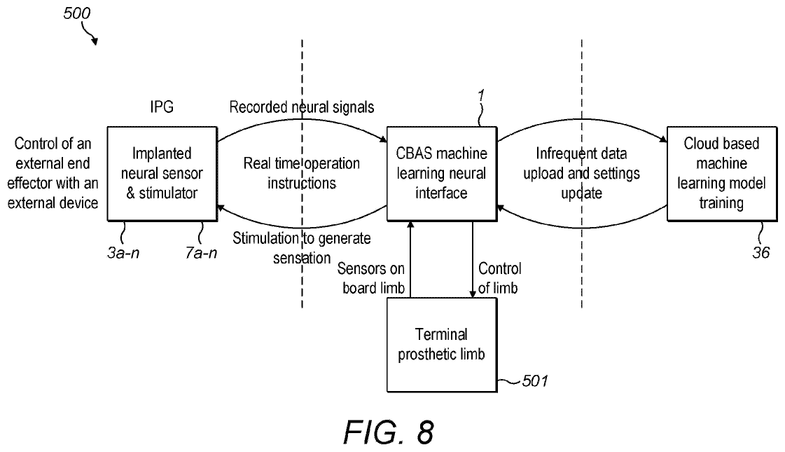

FIG. 8 shows a second example of a configuration of a system 500 incorporating the neural control system 1 to control an external device, such as a prosthetic limb device 501. In the second example the neural sensors 3a to 3n and stimulators 7a to 7n are implanted in the body of the subject, the neural control system 1 and the prosthetic limb device 501 are located externally of the subject, and the cloud servers 36 are located remotely from the neural control system 1 and the subject. In the second example the neural stimulators 7a to 7n may be used to provide a user with sensation corresponding to movement of the prosthetic limb device 36.

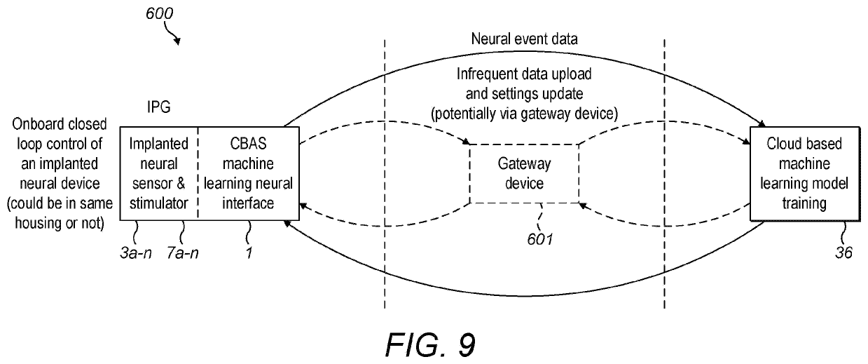

FIG. 9 shows a third example of a configuration of a system 600 incorporating the neural control system 1 to control an implanted neural device. In the first example the implanted neural device comprising neural sensors 3a to 3n and stimulators 7a to 7n are implanted in the body of the subject, the neural control system 1 is also implanted in the body of the subject, and the cloud servers 36 are located remotely from the neural control system 1 and the subject. In the system 600 of FIG. 9 the neural control system 1 is communicatively connected to the cloud servers 36 through a gateway device 601 located externally of the subject.

In the illustrated examples the neural control system 1 is used to provide control outputs to an end effector such as a neural stimulator and an external device such as a prosthetic limb device. Examples of possible end effector devices which can be controlled by the neural control system could comprise one, some, or all of: peripheral or central neural stimulation devices; devices that dispense nutrients; devices that dispense pharmaceuticals; devices that administer gene therapies, for example CRISPR; devices that administer viral vector treatments. This listing of possible end effector devices is not intended to be exhaustive. The end effector devices may be implanted inside the body of the subject, or they may be located external to the body of the subject, as appropriate. In some examples both internally implanted and external end effector devices may be controlled by the neural control system.

In the examples described above the neural control system 1 the neural control system outputs control signals to neural stimulators 7a to 7n and/or output devices 9a to 9n. In alternative examples the neural control system 1 may instead output the identified neural biomarkers identified by the ML neural processing, such as the ML data processing 103. The output identified neural biomarkers may, for example, be sent to a device able to process the neural biomarkers and use them as the basis for neural stimulation signals to be applied to the subjects nervous system, and/or control signals for an end effector or other device.

In some examples a targeted neural stimulus site to be stimulated by a neural stimulator may be treated with a viral vector or pharmaceutical agent arranged to enable hypersensitivity or hyposensitivity to neurostimulation.

As discussed above, in some examples neural sensors and stimulators may be implanted in a subject body, with the processing by the neural control system 1 being carried out outside the subject body.

In other examples, both the neural sensors and stimulators and the neural control system 1 may be implanted in a subject body.

In some examples the neural control system 1 may be used to carry out autonomic control of bodily conditions. In some examples the neural control system 1 may be used to carry out PID control. In some examples the neural control system 1 may be used to control a bionic organ.

In some examples the implanted neural data gathering and neural stimulation may be carried out entirely on the same device and electrodes, on the same chip with different electrode contacts, different electrodes and different chips but with chips or electrodes housed in the same casing, or entirely separate. The infrequent upload to the cloud may be by way of a local base station connected to when at home or during charging or may be a direct connection over cellular or wifi data connections.

In some examples the neural sensors and neural stimulators may be arranged to operate using different modalities in order to eliminate cross-talk between the neural stimulators and the neural sensors. For example, the neural sensors can operate by electrical sensing while the neural stimulators operate by optogenetic stimulation.

In the illustrated examples a single storage device 19 is used to store all of the raw signal data 21, processed signal data 23, classified signal data 26 and control signal data 29. In alternative examples the single storage device may be replaced by multiple storage devices. In particular, in some examples there may be a dedicated storage device or devices for each of the raw signal data, processed signal data, classified signal data, and control signal data.

In the illustrated example of FIG. 6 the stimulus encoding ML model may be carried out by a machine learning output processor. in other examples a dedicated machine learning output processor may be used to carry out other machine learning tasks to generate suitable output signals.

In the illustrated examples the modules of the system are defined in software. In other examples the modules may be defined wholly or in part in hardware, for example by dedicated electronic circuits.

In the illustrated examples the neural control system is formed as a unitary device. This is not essential, in other examples the neural control system may be formed as a distributed system.

In the described examples the system elements may be implemented as any form of a computing and/or electronic device.

Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

The computer executable instructions may be provided using any computer-readable media that is accessible by computing based device. Computer-readable media may include, for example, computer storage media such as a memory and communications media. Computer storage media, such as a memory, includes volatile and non-volatile, removable and non-removable media implemented in any method or technology for storage of information such as computer readable instructions, data structures, program modules or other data. Computer storage media includes, but is not limited to, RAM, ROM, EPROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device. In contrast, communication media may embody computer readable instructions, data structures, program modules, or other data in a modulated data signal, such as a carrier wave, or other transport mechanism. As defined herein, computer storage media does not include communication media.

The system 1 may be distributed or located remotely and accessed via a network or other communication link (e.g. using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of preferred embodiments is given by way of example only and that various modifications may be made by those skilled in the art. Although various embodiments have been described above with a certain degree of particularity, or with reference to one or more individual embodiments, those skilled in the art could make numerous alterations to the disclosed embodiments without departing from the spirit or scope of this invention.

The following examples are illustrative only and may be combined with aspects of other embodiments or teachings described herein, without limitation.

Example 1 is a neural control system comprising:

an input controller arranged to receive neural data regarding neural signals relating to a bodily state of a subject from at least one neural sensor;
at least one machine learning means using at least one machine learning model to process the received neural data to determine at least one output signal required to achieve a desired value of the bodily state; and
means arranged to send the determined output signal to at least one output device;
whereby the neural control system forms a first control loop providing closed loop control of the bodily state.

Example 2 is the system of example 1, wherein the at least one machine learning model comprises a single machine learning model for processing the received neural data to directly determine the at least one output signal.

Example 3 is the system of example 1, wherein the at least one machine learning model comprises:

a first machine learning model for processing the received neural data to identify neural biomarkers; and
a second machine learning model to process the identified neural biomarkers and determine the at least one output signal.

Example 4 is the system of example 1, wherein the at least one machine learning model comprises:

a first machine learning model for processing the received neural data to identify neural biomarkers; and

a closed loop controller receiving the neural biomarkers describing bodily state alongside the desired bodily setpoint to determine the output signal.

Example 5 is the system of example 1, wherein the at least one machine learning model comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 6 is the system of example 4 or 5, wherein the desired bodily setpoint is calculated within the closed loop controller based on received neural data or received neural biomarkers.

Example 7 is the system of example 5 or 6, wherein the neural control system uses the received data regarding bodily state of the subject from a non-neural sensor to calculate the desired bodily setpoint.

Example 8 is the system of example 4 or 5, wherein the closed loop controller uses the output of a body model to inform the decision of desired change in bodily state.

Example 9 is the system of example 8 wherein the body model is a state space model informed by any combination of neural biomarkers and/or non-neural sensors.

Example 10 is the system of example 8 wherein the body model is a functional model informed by any combination of neural biomarkers or non-neural sensors.

Example 11 is the system of example 8, 9, or 10 wherein the body model is updated based on received neural or other sensor data describing the bodily state of the subject subsequent to the applied output signal.

Example 12 is the system of example 11 wherein the body model update makes its own estimate of the response to the output signal and uses the comparison of this to the received neural or other sensor data to calculate the update to the body model.

Example 13 is the system of any preceding example, wherein the neural control system further comprises means arranged to receive data regarding a bodily state of the subject from a non-neural sensor;

wherein the at least one machine learning means further processes the received data to determine the at least one output signal.

Example 14 is the system of any preceding example, wherein the neural control system further comprises an output controller arranged to receive the determined output signal and to send the determined output signal to the at least one output device.

Example 15 is the system of example 14, wherein the output controller is arranged to receive selected data regarding received neural data from the input controller;

whereby the input controller and the output controller form a second control loop having a shorter response latency than the first control loop.

Example 16 is the system of example 15, wherein the output controller is arranged to carry out real time modulation of any parameters of the determined output signal sent to the at least one output device based at least in part on the selected data characterizing the received neural data.

Example 17 is the system of example 16, wherein the real time modulation happens in 1 to 100 microseconds.

Example 18 is the system of any one of examples 15 to 17, wherein the output controller is arranged to receive selected data regarding timing of received neural data from the input controller;

wherein the output controller is arranged to control the time at which the determined output signal is sent based at least in part on the selected data regarding timing of received neural data.

Example 19 is the system of any one of examples 15 to 17, wherein the output controller is arranged to receive selected data regarding amplitude of received neural data from the input controller;

wherein the output controller is arranged to control the amplitude and/or frequency of the determined output signal sent to the at least one output device based at least in part on the selected data regarding amplitude of received neural data.

Example 20 is the system of any preceding example, wherein the output signal is a control signal of an end effector device.

Example 21 is the system of any preceding example, wherein the output signal is a neural stimulation signal to be applied to the nervous system of the subject.

Example 22 is the system of any preceding example, wherein the output signal is a control signal for an applied drug treatment.

Example 23 is the system of example 21, wherein the neural control system further comprises an output controller arranged to receive the determined output neural stimulation signal and to send the determined output neural stimulation signal to the at least one output device, and to send data regarding the timing of the determined output neural stimulation signal to the input controller.

Example 24 is the system of example 23, wherein the input controller is arranged to stop sending the received neural data to the at least one machine learning means when the determined output neural stimulation signal is being output.

Example 25 is the system of any preceding example, wherein the neural control system further comprises means for sending the received neural data to an update machine.

Example 26 is the system of any preceding example wherein the neural control system further comprises means for sending any combination of; the record of calculated neural biomarkers, output signals, recorded neural data, data from other sensors, or data representing bodily state to the update machine.

Example 27 is the system of any preceding example, wherein the neural control system further comprises means for receiving updates of the at least one machine learning model.

Example 28 is the system of example 27, wherein the updates to the at least one machine learning model are calculated based on the received neural data, calculated neural biomarkers, output signals, recorded neural data, data from other sensors, or data representing bodily state.

Example 29 is the system of any preceding example, wherein the neural control system further comprises means for receiving updates to the ideal bodily setpoint or calculation of bodily setpoint as a means of updating the applied treatment.

Example 30 is the system of any of examples 8 to 12, wherein the neural control system further comprises means for receiving updates to the body model.

Example 31 is the system of examples 26 to 30 wherein the updates are generated based on data recorded during specific periods of guided activity during rehabilitation or recalibration periods.

Example 32 is the system of example 26 to 30 wherein the update machine is an automated cloud system.

Example 33 is the system of example 26 to 30 wherein the update machine is a manual connection over a local wired or wireless connection.

Example 34 is the system of examples 26 to 30 wherein the updates are automatically calculated by one or more machine learning systems for calculating long term treatment.

Example 35 is the system of examples 26 to 30 wherein the updates are chosen by a treating clinician.

Example 36 is the system of any preceding example, wherein the neural control system incorporates a safety module for monitoring performance of the neural control system.

Example 37 is the system of example 36, wherein the neural control system comprises one or more elements from the group of:

Machine learning data processor;

Control module;

Real time input controller;

Real time output controller;

Stimulation library selector;

Machine learning output processor;

Bodily setpoint calculator; and

Body model;

wherein the performance of these elements is monitored by the safety module.

Example 38 is the system of example 36 or 37, wherein the safety module further monitors the performance of the real time input controller and output controller within the real time loop.

Example 39 is the system of examples 36 to 38, wherein the safety module is arranged to act to selectively reduce or stop the function of any element of the neural control system based on monitoring of performance of that element.

Example 40 is the system of examples 36 to 39, wherein the safety module is arranged to selectively control the neural control system to operate in a safety mode.

Example 41 is the system of examples 36 to 40, wherein the neural control system further comprises an auditing module arranged to monitor version control of the neural control system; and the safety module is arranged to act based on commands from the auditing module.

Example 42 is the system of any preceding example, wherein the neural control system further comprises an auditing module arranged to monitor version control of the neural control system.

Example 43 is the system of example 41 or 42, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements.

Example 44 is the system of example 42 or 43, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control system of these elements.

Example 45 is the system of any one of examples 42 to 44, wherein the auditing module is arranged to command the safety module to act to reduce or stop the function of the neural control system based on the auditing module detecting at least one of:

Incorrect versioning

Out of date versioning

Unauthorised versioning

Example 46 is the system of any preceding example in which the at least one neural sensor operates using a first modality and the output signal is a neural stimulation signal to be applied to the nervous system of the subject using a second modality different from the first.

Example 47 is a neural control system comprising:

an input controller arranged to receive neural data regarding neural signals relating to a bodily state from at least one neural sensor;

at least one machine learning model for processing the received neural data to identify neural biomarkers; and

means arranged to send the identified neural biomarkers to at least one output device for performing operations;

wherein the neural control system forms a first control loop providing closed loop control of the bodily state.

Example 48 is the system of example 47, wherein the neural biomarkers are learnt over a population of patients to be stationary representations of neural population activity.

Example 49 is the system of example 47 or 48, wherein the neural biomarkers are learnt over a population of patients to be stationary representations of neural population activity.

Example 50 is the system of any of examples 47 to 49, wherein the neural control system further comprises means for receiving updates of the at least one machine learning model.

Example 51 is the system of example 50, wherein the updates to the at least one machine learning model are calculated based on the received neural data calculated neural biomarkers, output signals, recorded neural data, data from other sensors, or data representing bodily state.

Example 52 is the system of any of examples 47 to 51, wherein the neural control system further comprises means for receiving updates to the ideal bodily setpoint or calculation of bodily setpoint as a means of updating the applied treatment.

Example 53 is the system of any of examples 47 to 52, wherein the neural control system further comprises means for receiving updates to the body model.

Example 54 is the system of examples 47 to 53 wherein the updates are generated based on data recorded during specific periods of guided activity during rehabilitation or recalibration periods.

Example 55 is the system of example 47 to 53 wherein the update machine is an automated cloud system.

Example 56 is the system of example 47 to 53 wherein the update machine is a manual connection over a local wired or wireless connection.

Example 57 is the system of examples 47 to 53 wherein the updates are automatically calculated by one or more machine learning systems for calculating long term treatment.

Example 58 is the system of examples 47 to 53 wherein the updates are chosen by a treating clinician.

Example 59 is the system of any one of examples 47 to 58, wherein the neural control system incorporates a safety module for monitoring performance of the neural control system.

Example 60 is the system of example 59, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 61 is the system of example 59 or 60 wherein the safety module is arranged to act to selectively reduce or stop the function of any element of the neural control system based on monitoring of performance of that element.

Example 62 is the system of examples 59 to 61, wherein the safety module is arranged to selectively control the neural control system to operate in a safety mode.

Example 63 is the system of examples 59 to 62, wherein the neural control system further comprises an auditing module arranged to monitor version control of the neural control system; and

the safety module is arranged to act based on commands from an auditing module.

Example 64 is the system of any one of examples 47 to 63, wherein the neural control system further comprises an auditing module arranged to monitor version control of the neural control system.

Example 65 is the system of example 63 or 64, the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements

Example 66 is the system of example 64 or 65, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control system of these elements.

Example 67 is the system of any one of examples 62 to 66, wherein the auditing module is arranged to command the safety module to act to reduce or stop the function of the neural control system based on the auditing module detecting at least one of:

Incorrect versioning

Out of date versioning

Unauthorised versioning

Example 68 is the system of example of any of examples 47 to 67, wherein the at least one neural sensor operates using a first modality and the output signal is a neural stimulation signal to be applied to the nervous system of the subject using a second modality different from the first.

Example 69 is a neural control system comprising:

an input controller arranged to receive neural data regarding neural signals relating to a bodily state in nerves of a subject from at least one neural sensor;

at least one neural data processing means arranged to process the received neural data to determine at least one output signal required to achieve a desired value of the bodily state; and

an output controller arranged to receive the determined output signal and to send the determined output signal to the at least one output device;

whereby the neural control system forms a first control loop providing closed loop control of the bodily state;
wherein the output controller is further arranged to receive selected data regarding timing of received neural data from the input controller; and
wherein the output controller is arranged to control the time at which the determined output signal is sent to the at least one output device based on the selected data regarding timing of received neural data.

Example 70 is the system of example 69, wherein the neural data processing means comprises a single machine learning model for processing the received neural data to directly determine the at least one output signal.

Example 71 is the system of example 69, wherein the neural data processing means comprises:

a first machine learning model for processing the received neural data to identify neural biomarkers; and
a second machine learning model to process the identified neural biomarkers and determine the at least one output signal.

Example 72 is the system of example 69, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 73 is the system of example 69, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 74 is the system of example 72 or 73, wherein the desired bodily setpoint is calculated within the closed loop controller based on received neural data or received neural biomarkers.

Example 75 is the system of example 73 or 74, wherein the neural control system uses the received data regarding bodily state of the subject from a non-neural sensor to calculate the desired bodily setpoint.

Example 76 is the system of example 72 or 73, wherein the closed loop controller uses the output of a body model to inform the decision of desired change in bodily state.

Example 77 is the system of example 76 wherein the body model is a state space model informed by any combination of neural biomarkers and/or non-neural sensors.

Example 78 is the system of example 76 wherein the body model is a functional model informed by any combination of neural biomarkers or non-neural sensors.

Example 79 is the system of examples 76 to 78 wherein the body model is updated based on received neural or other sensor data describing the bodily state of the subject subsequent to the applied output signal.

Example 80 is the system of example 79 wherein the body model update makes its own estimate of the response to the output signal and uses the comparison of this to the received neural or other sensor data to calculate the update to the body model.

Example 81 is the system of any of examples 69 to 80, wherein the neural control system further comprises means arranged to receive data regarding a bodily state of the subject from a non-neural sensor;

wherein the at least one machine learning means further processes the received data to determine the at least one output signal.

Example 82 is the system of any of examples 69 to 81, wherein the output controller is arranged to receive selected data regarding received neural data from the input controller;

whereby the input controller and the output controller form a second control loop having a shorter response latency than the first control loop.

Example 83 is the system of example 82, wherein the output controller is arranged to carry out real time modulation of any parameters of the determined output signal sent to the at least one output device based at least in part on the selected data characterizing the received neural data.

Example 84 is the system of example 83, wherein the real time modulation happens in 1 to 100 microseconds.

Example 85 is the system of any one of examples 82 to 84, wherein the output controller is arranged to receive selected data regarding timing of received neural data from the input controller;

wherein the output controller is arranged to control the time at which the determined output signal is sent based at least in part on the selected data regarding timing of received neural data.

Example 86 is the system of any one of examples 83 to 85, wherein the output controller is arranged to receive selected data regarding amplitude of received neural data from the input controller;

wherein the output controller is arranged to control the amplitude and/or frequency of the determined output signal sent to the at least one output device based at least in part on the selected data regarding amplitude of received neural data.

Example 87 is the system of any of examples 70 to 86, wherein the output signal is a control signal of an end effector device.

Example 88 is the system of any of examples 70 to 87, wherein the output signal is a neural stimulation signal to be applied to the nervous system of the subject.

Example 89 is the system of any of examples 70 to 88, wherein the output signal is a control signal for an applied drug treatment.

Example 90 is the system of example 88, wherein the neural control system further comprises an output controller arranged to receive the determined output neural stimulation signal and to send the determined output neural stimulation signal to the at least one output device, and to send data regarding the timing of the determined output neural stimulation signal to the input controller.

Example 91 is the system of example 90, wherein the input controller is arranged to stop sending the received neural data to the at least one machine learning means when the determined output neural stimulation signal is being output.

Example 92 is the system of any of examples 69 to 91, wherein the neural control system further comprises means for sending the received neural data to an update machine.

Example 93 is the system of any of examples 69 to 92, wherein the neural control system further comprises means for sending any combination of; the record of calculated neural biomarkers, output signals, recorded neural data, data from other sensors, or data representing bodily state to the update machine.

Example 94 is the system of any of examples 69 to 93, wherein the neural control system further comprises means for receiving updates of the at least one machine learning model.

Example 95 is the system of example 94, wherein the updates to the at least one machine learning model are calculated based on the received neural data calculated neural biomarkers, output signals, recorded neural data, data from other sensors, or data representing bodily state.

Example 96 is the system of any of examples 69 to 95, wherein the neural control system further comprises means for receiving updates to the ideal bodily setpoint or calculation of bodily setpoint as a means of updating the applied treatment.

Example 97 is the system of any of examples 69 to 96, wherein the neural control system further comprises means for receiving updates to the body model.

Example 98 is the system of examples 94 to 97 wherein the updates are generated based on data recorded during specific periods of guided activity during rehabilitation or recalibration periods.

Example 99 is the system of example 94 to 98 wherein the update machine is an automated cloud system.

Example 100 is the system of example 94 to 98 wherein the update machine is a manual connection over a local wired or wireless connection.

Example 101 is the system of examples 94 to 98 wherein the updates are automatically calculated by one or more machine learning systems for calculating long term treatment.

Example 102 is the system of examples 94 to 98 wherein the updates are chosen by a treating clinician.

Example 103 is the system of any of examples 69 to 102 wherein the neural control system incorporates a safety module for monitoring performance of the neural control system.

Example 104 is the system of example 103 wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 105 is the system of example 103 or 104 wherein the safety module further monitors the performance of the real time input controller and output controller within the real time loop.

Example 106 is the system of examples 103 to 105 wherein the safety module is arranged to act to selectively reduce or stop the function of any element of the neural control system based on monitoring of performance of that element.

Example 107 is the system of examples 103 to 106 wherein the safety module is arranged to selectively control the neural control system to operate in a safety mode.

Example 108 is the system of examples 103 to 107, wherein the neural control system further comprises an auditing module arranged to monitor version control of the neural control system; and

the safety module is arranged to act based on commands from the auditing module.

Example 109 is the system of any of examples 69 to 108, wherein the neural control system further comprises an auditing module arranged to monitor version control of the neural control system.

Example 110 is the system of example 108 or 109, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements.

Example 111 is the system of example 109 or 110, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control system of these elements.

Example 112 is the system of any one of examples 109 to 111, wherein the auditing module is arranged to command the safety module to act to reduce or stop the function of the neural control system based on the auditing module detecting at least one of:

Incorrect versioning

Out of date versioning

Unauthorised versioning

Example 113 is the system of any of examples 69 to 112 in which the at least one neural sensor operates using a first modality and the output signal is a neural stimulation signal to be applied to the nervous system of the subject using a second modality different from the first.

Example 114 is a neural control system comprising:

an input controller arranged to receive neural data regarding neural signals relating to a bodily state in nerves of a subject from at least one neural sensor;
at least one neural data processing means arranged to process the received neural data to determine at least one output signal required to achieve a desired value of the bodily state; and
an output controller arranged to receive the determined output signal and to send the determined output signal to the at least one output device;
whereby the neural control system forms a first control loop providing closed loop control of the bodily state;
wherein the output controller is further arranged to receive selected data regarding amplitude of received neural data from the input controller; and
wherein the output controller is arranged to control the amplitude of the determined output signal sent to the at least one output device based on the selected data regarding amplitude of received neural data.

Example 115 is the system of example 114, wherein the neural data processing means comprises a single machine learning model for processing the received neural data to directly determine the at least one output signal.

Example 116 is the system of example 114, wherein the neural data processing means comprises:

a first machine learning model for processing the received neural data to identify neural biomarkers; and
a second machine learning model to process the identified neural biomarkers and determine the at least one output signal.

Example 117 is the system of example 114, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 118 is the system of example 114, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 119 is the system of example 117 or 118, wherein the desired bodily setpoint is calculated within the closed loop controller based on received neural data or received neural biomarkers.

Example 120 is the system of example 118 or 119, wherein the neural control system uses the received data regarding bodily state of the subject from a non-neural sensor to calculate the desired bodily setpoint.

Example 121 is the system of example 117 or 118, wherein the closed loop controller uses the output of a body model to inform the decision of desired change in bodily state.

Example 122 is the system of example 121 wherein the body model is a state space model informed by any combination of neural biomarkers and/or non-neural sensors.

Example 123 is the system of example 121 wherein the body model is a functional model informed by any combination of neural biomarkers or non-neural sensors.

Example 124 is the system of examples 121 to 123 wherein the body model is updated based on received neural or other sensor data describing the bodily state of the subject subsequent to the applied output signal.

Example 125 is the system of example 125 wherein the body model update makes its own estimate of the response to the output signal and uses the comparison of this to the received neural or other sensor data to calculate the update to the body model.

Example 126 is the system of any of examples 114 to 125, wherein the neural control system further comprises means arranged to receive data regarding a bodily state of the subject from a non-neural sensor;

wherein the at least one machine learning means further processes the received data to determine the at least one output signal.

Example 127 is the system of any of examples 114 to 126, wherein the output controller is arranged to receive selected data regarding received neural data from the input controller;

whereby the input controller and the output controller form a second control loop having a shorter response latency than the first control loop.

Example 128 is the system of example 127, wherein the output controller is arranged to carry out real time modulation of any parameters of the determined output signal sent to the at least one output device based at least in part on the selected data characterizing the received neural data.

Example 129 is the system of example 128, wherein the real time modulation happens in 1 to 100 microseconds.

Example 130 is the system of any one of examples 127 to 129, wherein the output controller is arranged to receive selected data regarding timing of received neural data from the input controller;

wherein the output controller is arranged to control the time at which the determined output signal is sent based at least in part on the selected data regarding timing of received neural data.

Example 131 is the system of any one of examples 127 to 129, wherein the output controller is arranged to receive selected data regarding amplitude of received neural data from the input controller;

wherein the output controller is arranged to control the frequency of the determined output signal sent to the at least one output device based at least in part on the selected data regarding amplitude of received neural data.

Example 132 is the system of any of examples 114 to 131, wherein the output signal is a control signal of an end effector device.

Example 133 is the system of any of examples 114 to 132, wherein the output signal is a neural stimulation signal to be applied to the nervous system of the subject.

Example 134 is the system of any of examples 114 to 133, wherein the output signal is a control signal for an applied drug treatment.

Example 135 is the system of example 134, wherein the neural control system further comprises an output controller arranged to receive the determined output neural stimulation signal and to send the determined output neural stimulation signal to the at least one output device, and to send data regarding the timing of the determined output neural stimulation signal to the input controller.

Example 136 is the system of example 135, wherein the input controller is arranged to stop sending the received neural data to the at least one machine learning means when the determined output neural stimulation signal is being output.

Example 137 is the system of any of examples 114 to 136, wherein the neural control system further comprises means for sending the received neural data to an update machine.

Example 138 is the system of any of examples 114 to 137, wherein the neural control system further comprises means for sending any combination of; the record of calculated neural biomarkers, output signals, recorded neural data, data from other sensors, or data representing bodily state to the update machine.

Example 139 is the system of any of examples 114 to 138, wherein the neural control system further comprises means for receiving updates of the at least one machine learning model.

Example 140 is the system of example 139, wherein the updates to the at least one machine learning model are calculated based on the received neural data calculated neural biomarkers, output signals, recorded neural data, data from other sensors, or data representing bodily state.

Example 141 is the system of any of examples 114 to 140, wherein the neural control system further comprises means for receiving updates to the ideal bodily setpoint or calculation of bodily setpoint as a means of updating the applied treatment.

Example 142 is the system of any of examples 114 to 141, wherein the neural control system further comprises means for receiving updates to the body model.

Example 143 is the system of examples 138 to 142 wherein the updates are generated based on data recorded during specific periods of guided activity during rehabilitation or recalibration periods.

Example 144 is the system of example 138 to 142 wherein the update machine is an automated cloud system.

Example 145 is the system of example 138 to 142 wherein the update machine is a manual connection over a local wired or wireless connection.

Example 146 is the system of examples 138 to 142 wherein the updates are automatically calculated by one or more machine learning systems for calculating long term treatment.

Example 147 is the system of examples 138 to 142 wherein the updates are chosen by a treating clinician.

Example 148 is the system of any of examples 114 to 147, wherein the neural control system incorporates a safety module for monitoring performance of the neural control system.

Example 149 is the system of example 148, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 150 is the system of example 148 or 149, wherein the safety module further monitors the performance of the real time input controller and output controller within the real time loop.

Example 151 is the system of examples 148 to 150, wherein the safety module is arranged to act to selectively reduce or stop the function of any element of the neural control system based on monitoring of performance of that element.

Example 152 is the system of examples 148 to 151, wherein the safety module is arranged to selectively control the neural control system to operate in a safety mode.

Example 153 is the system of examples 148 to 152, wherein the neural control system further comprises an auditing module arranged to monitor version control of the neural control system; and

the safety module is arranged to act based on commands from the auditing module.

Example 154 is the system of any of examples 114 to 153, wherein the neural control system further comprises an auditing module arranged to monitor version control of the neural control system.

Example 155 is the system of example 153 or 154, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements.

Example 156 is the system of example 154 or 155, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control system of these elements.

Example 157 is the system of any one of examples 154 to 156, wherein the auditing module is arranged to command the safety module to act to reduce or stop the function of the neural control system based on the auditing module detecting at least one of:

Incorrect versioning

Out of date versioning

Unauthorised versioning

Example 158 is the system of example of any of examples 114 to 157 in which the at least one neural sensor operates using a first modality and the output signal is a neural stimulation signal to be applied to the nervous system of the subject using a second modality different from the first.

Example 159 is a neural control system comprising:

an input controller arranged to receive neural data regarding neural signals relating to a bodily state in nerves of a subject from at least one neural sensor;
at least one neural data processing means arranged to process the received neural data to determine at least one output neural stimulation signal required to achieve a desired value of the bodily state; and
an output controller arranged to receive the determined output neural stimulation signal and to send the determined output neural stimulation signal to the at least one output device;
whereby the neural control system forms a first control loop providing closed loop control of the bodily state;
wherein the output controller is further arranged to receive selected data regarding amplitude of received neural data from the input controller; and
wherein the output controller is further arranged to send data regarding the timing of the determined output neural stimulation signal to the input controller; and
the input controller is arranged to stop sending the received neural data to the at least one machine learning means when the determined output neural stimulation signal is being output.

Example 160 is the system of example 159, wherein the neural data processing means comprises a single machine learning model for processing the received neural data to directly determine the at least one output signal.

Example 161 is the system of example 159, wherein the neural data processing means comprises:

a first machine learning model for processing the received neural data to identify neural biomarkers; and
a second machine learning model to process the identified neural biomarkers and determine the at least one output signal.

Example 162 is the system of example 159, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 163 is the system of example 159, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 164 is the system of example 162 or 163, wherein the desired bodily setpoint is calculated within the closed loop controller based on received neural data or received neural biomarkers.

Example 165 is the system of example 163 or 164, wherein the neural control system uses the received data regarding bodily state of the subject from a non-neural sensor to calculate the desired bodily setpoint.

Example 166 is the system of example 162 or 163, wherein the closed loop controller uses the output of a body model to inform the decision of desired change in bodily state.

Example 167 is the system of example 166 wherein the body model is a state space model informed by any combination of neural biomarkers and/or non-neural sensors.

Example 168 is the system of example 166 wherein the body model is a functional model informed by any combination of neural biomarkers or non-neural sensors.

Example 169 is the system of examples 166 to 168 wherein the body model is updated based on received neural or other sensor data describing the bodily state of the subject subsequent to the applied output signal.

Example 170 is the system of example 169 wherein the body model update makes its own estimate of the response to the output signal and uses the comparison of this to the received neural or other sensor data to calculate the update to the body model.

Example 171 is the system of any of examples 159 to 170, wherein the neural control system further comprises means arranged to receive data regarding a bodily state of the subject from a non-neural sensor;

wherein the at least one machine learning means further processes the received data to determine the at least one output signal.

Example 172 is the system of any of examples 159 to 171, wherein the output controller is arranged to receive selected data regarding received neural data from the input controller;

whereby the input controller and the output controller form a second control loop having a shorter response latency than the first control loop.

Example 173 is the system of example 172, wherein the output controller is arranged to carry out real time modulation of any parameters of the determined output signal sent to the at least one output device based at least in part on the selected data characterizing the received neural data.

Example 174 is the system of example 173, wherein the real time modulation happens in 1 to 100 microseconds.

Example 175 is the system of any one of examples 172 to 174, wherein the output controller is arranged to receive selected data regarding timing of received neural data from the input controller;

wherein the output controller is arranged to control the time at which the determined output signal is sent based at least in part on the selected data regarding timing of received neural data.

Example 176 is the system of any one of examples 172 to 174, wherein the output controller is arranged to receive selected data regarding amplitude of received neural data from the input controller;

wherein the output controller is arranged to control the amplitude and/or frequency of the determined output signal sent to the at least one output device based at least in part on the selected data regarding amplitude of received neural data.

Example 177 is the system of any of examples 159 to 176, wherein the output signal is a control signal of an end effector device.

Example 178 is the system of any of examples 159 to 177, wherein the output signal is a neural stimulation signal to be applied to the nervous system of the subject.

Example 179 is the system of any of examples 159 to 178, wherein the output signal is a control signal for an applied drug treatment.

Example 180 is the system of example 178, wherein the output controller is arranged to send data regarding the timing of the determined output neural stimulation signal to the input controller.

Example 181 is the system of any of examples 159 to 180, wherein the neural control system further comprises means for sending the received neural data to an update machine.

Example 182 is the system of any of examples 159 to 181, wherein the neural control system further comprises means for sending any combination of; the record of calculated neural biomarkers, output signals, recorded neural data, data from other sensors, or data representing bodily state to the update machine.

Example 183 is the system of any of examples 159 to 182, wherein the neural control system further comprises means for receiving updates of the at least one machine learning model.

Example 184 is the system of example 183, wherein the updates to the at least one machine learning model are calculated based on the received neural data calculated neural biomarkers, output signals, recorded neural data, data from other sensors, or data representing bodily state.

Example 185 is the system of any of examples 159 to 184, wherein the neural control system further comprises means for receiving updates to the ideal bodily setpoint or calculation of bodily setpoint as a means of updating the applied treatment.

Example 186 is the system of any of examples 159 to 185, wherein the neural control system further comprises means for receiving updates to the body model.

Example 187 is the system of examples 183 to 186 wherein the updates are generated based on data recorded during specific periods of guided activity during rehabilitation or recalibration periods.

Example 188 is the system of example 183 to 187 wherein the update machine is an automated cloud system.

Example 189 is the system of example 183 to 187 wherein the update machine is a manual connection over a local wired or wireless connection.

Example 190 is the system of examples 183 to 187 wherein the updates are automatically calculated by one or more machine learning systems for calculating long term treatment.

Example 191 is the system of examples 183 to 187 wherein the updates are chosen by a treating clinician.

Example 192 is the system of any of examples 159 to 191, wherein the neural control system incorporates a safety module for monitoring performance of the neural control system.

Example 193 is the system of example 192, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 194 is the system of example 192 or 193, wherein the safety module further monitors the performance of the real time input controller and output controller within the real time loop.

Example 195 is the system of examples 192 to 194, wherein the safety module is arranged to act to selectively reduce or stop the function of any element of the neural control system based on monitoring of performance of that element.

Example 196 is the system of examples 192 to 195, wherein the safety module is arranged to selectively control the neural control system to operate in a safety mode.

Example 197 is the system of examples 192 to 196, wherein the neural control system further comprises an auditing module arranged to monitor version control of the neural control system; and

the safety module is arranged to act based on commands from the auditing module.

Example 198 is the system of any of examples 159 to 197, wherein the neural control system further comprises an auditing module arranged to monitor version control of the neural control system.

Example 199 is the system of example 197 or 198, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements.

Example 200 is the system of example 198 or 199, wherein the the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control system of these elements.

Example 201 is the system of any one of examples 198 to 200, wherein the auditing module is arranged to command the safety module to act to reduce or stop the function of the neural control system based on the auditing module detecting at least one of:

Incorrect versioning

Out of date versioning

Unauthorised versioning

Example 202 is the system of example of any of examples 159 to 201 in which the at least one neural sensor operates using a first modality and the output signal is a neural stimulation signal to be applied to the nervous system of the subject using a second modality different from the first.

Example 203 is a neural control system comprising:

an input controller arranged to receive neural data regarding neural signals relating to a bodily state from at least one neural sensor;

at least one machine learning means using at least one machine learning model to process the received neural data to determine at least one output signal required to achieve a desired value of the bodily state; and

means arranged to send the determined output signal to at least one output device;
whereby the neural control system forms a first control loop providing closed loop control of the bodily state; and

wherein the neural control system is connected to a separate update machine, including means for sending the received neural data to the update machine; wherein the update machine is arranged to use the received neural data to perform machine learning training to produce an updated machine learning model; and

the neural control system is further arranged to replace the at least one machine learning model with the updated machine learning model.

Example 204 is the system of example 203, wherein the uploaded neural data is used to retrain the machine learning means.

Example 205 is the system of example 203 or 204, wherein the at least one machine learning means is used to calculate neural biomarkers.

Example 206 is the system of examples 203 to 205, wherein the at least one machine learning means calculating neural biomarkers is updated based on a machine learning retraining from neural data representative of one or more patients across one or more time periods.

Example 207 is the system of examples 204 to 205, wherein the at least one machine learning model comprises a single machine learning model for processing the received neural data to directly determine the at least one output signal.

Example 208 is the system of examples 203 to 205, wherein the at least one machine learning model comprises:

a first machine learning model for processing the received neural data to identify neural biomarkers; and
a second machine learning model to process the identified neural biomarkers and determine the at least one output signal.

Example 209 is the system of examples 204 to 206, wherein the at least one machine learning model comprises:

a first machine learning model for processing the received neural data to identify neural biomarkers; and

a closed loop controller receiving the neural biomarkers describing bodily state alongside the desired bodily setpoint to determine the output signal.

Example 210 is the system of examples 203 to 205, wherein the at least one machine learning model comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 211 is the system of example 209 or 210, wherein the desired bodily setpoint is calculated within the closed loop controller based on received neural data or received neural biomarkers.

Example 212 is the system of example 210 or 211, wherein the neural control system uses the received data regarding bodily state of the subject from a non-neural sensor to calculate the desired bodily setpoint.

Example 213 is the system of example 209 or 210, wherein the closed loop controller uses the output of a body model to inform the decision of desired change in bodily state.

Example 214 is the system of example 213 wherein the body model is a state space model informed by any combination of neural biomarkers and/or non-neural sensors.

Example 215 is the system of example 213 wherein the body model is a functional model informed by any combination of neural biomarkers or non-neural sensors.

Example 216 is the system of example 213 to 215 wherein the body model is updated based on received neural or other sensor data describing the bodily state of the subject subsequent to the applied output signal.

Example 217 is the system of example 216 wherein the body model update makes its own estimate of the response to the output signal and uses the comparison of this to the received neural or other sensor data to calculate the update to the body model.

Example 218 is the system of examples 203 to 217, wherein the neural control system further comprises means arranged to receive data regarding a bodily state of the subject from a non-neural sensor;

wherein the at least one machine learning means further processes the received data to determine the at least one output signal.

Example 219 is the system of examples 203 to 218, wherein the neural control system further comprises an output controller arranged to receive the determined output signal and to send the determined output signal to the at least one output device.

Example 220 is the system of example 219, wherein the output controller is arranged to receive selected data regarding received neural data from the input controller;

whereby the input controller and the output controller form a second control loop having a shorter response latency than the first control loop.

Example 221 is the system of example 220, wherein the output controller is arranged to carry out real time modulation of any parameters of the determined output signal sent to the at least one output device based at least in part on the selected data characterizing the received neural data.

Example 222 is the system of example 221, wherein the real time modulation happens in 1 to 100 microseconds.

Example 223 is the system of any one of examples 220 to 222, wherein the output controller is arranged to receive selected data regarding timing of received neural data from the input controller;

wherein the output controller is arranged to control the time at which the determined output signal is sent based at least in part on the selected data regarding timing of received neural data.

Example 224 is the system of any one of examples 220 to 222, wherein the output controller is arranged to receive selected data regarding amplitude of received neural data from the input controller;

wherein the output controller is arranged to control the amplitude and/or frequency of the determined output signal sent to the at least one output device based at least in part on the selected data regarding amplitude of received neural data.

Example 225 is the system of any of examples 203 to 224, wherein the output signal is a control signal of an end effector device.

Example 226 is the system of examples 203 to 225, wherein the output signal is a neural stimulation signal to be applied to the nervous system of the subject.

Example 227 is the system of any of examples 203 to 226, wherein the output signal is a control signal for an applied drug treatment.

Example 228 is the system of example 227, wherein the neural control system further comprises an output controller arranged to receive the determined output neural stimulation signal and to send the determined output neural stimulation signal to the at least one output device, and to send data regarding the timing of the determined output neural stimulation signal to the input controller.

Example 229 is the system of example 228, wherein the input controller is arranged to stop sending the received neural data to the at least one machine learning means when the determined output neural stimulation signal is being output.

Example 230 is the system of any of examples 203 to 229, wherein the neural control system further comprises means for sending the received neural data to an update machine.

Example 231 is the system of any of examples 203 to 230, wherein the neural control system further comprises means for sending any combination of; the record of calculated neural biomarkers, output signals, recorded neural data, data from other sensors, or data representing bodily state to the update machine.

Example 232 is the system of any of examples 203 to 231, wherein the neural control system further comprises means for receiving updates of the at least one machine learning model.

Example 233 is the system of example 232, wherein the updates to the at least one machine learning model are calculated based on the received neural data calculated neural biomarkers, output signals, recorded neural data, data from other sensors, or data representing bodily state.

Example 234 is the system of any of examples 203 to 233, wherein the neural control system further comprises means for receiving updates to the ideal bodily setpoint or calculation of bodily setpoint as a means of updating the applied treatment.

Example 235 is the system of any of examples 213 to 217, wherein the neural control system further comprises means for receiving updates to the body model.

Example 236 is the system of examples 231 to 235 wherein the updates are generated based on data recorded during specific periods of guided activity during rehabilitation or recalibration periods.

Example 237 is the system of example 231 to 235 wherein the update machine is an automated cloud system.

Example 238 is the system of example 231 to 235 wherein the update machine is a manual connection over a local wired or wireless connection.

Example 239 is the system of examples 231 to 235 wherein the updates are automatically calculated by one or more machine learning systems for calculating long term treatment.

Example 240 is the system of examples 231 to 235 wherein the updates are chosen by a treating clinician.

Example 241 is the system of examples 203 to 240, wherein the neural control system incorporates a safety module for monitoring performance of the neural control system.

Example 242 is the system of example 241, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 243 is the system of example 241 or 242, wherein the safety module further monitors the performance of the real time input controller and output controller within the real time loop.

Example 244 is the system of examples 241 to 243, wherein the safety module is arranged to act to selectively reduce or stop the function of any element of the neural control system based on monitoring of performance of that element.

Example 245 is the system of examples 241 to 244, wherein the safety module is arranged to selectively control the neural control system to operate in a safety mode.

Example 246 is the system of examples 241 to 245, wherein the neural control system further comprises an auditing module arranged to monitor version control of the neural control system; and

the safety module is arranged to act based on commands from the auditing module.

Example 247 is the system of examples 203 to 246, wherein the neural control system further comprises an auditing module arranged to monitor version control of the neural control system.

Example 248 is the system of example 246 or 247, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements.

Example 249 is the system of example 247 or 248, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control system of these elements.

Example 250 is the system of any one of examples 247 to 249, wherein the auditing module is arranged to command the safety module to act to reduce or stop the function of the neural control system based on the auditing module detecting at least one of:

Incorrect versioning

Out of date versioning

Unauthorised versioning.

Example 251 is the system of examples 203 to 251, in which the at least one neural sensor operates using a first modality and the output signal is a neural stimulation signal to be applied to the nervous system of the subject using a second modality different from the first.

Example 252 is a neural control system comprising:

an input controller arranged to receive neural data regarding neural signals relating to a bodily state from at least one neural sensor;

at least one neural data processing means arranged to process the received neural data to determine at least one output signal required to achieve a desired value of the bodily state; and

means arranged to send the determined output signal to at least one output device;

whereby the neural control system forms a first control loop providing closed loop control of the bodily state; and

wherein the neural control system incorporates a safety module monitoring performance of the closed loop controller;

wherein the safety module is arranged to act to reduce or stop the function of any element of the first control loop based on monitoring its performance.

Example 253 is the system of example 252, wherein the safety module is arranged to act to reduce or stop the function of any element of the neural control system based on monitoring its performance.

Example 254 is the system of example 252 or 253, wherein the neural data processing means comprises a single machine learning model for processing the received neural data to directly determine the at least one output signal.

Example 255 is the system of examples 252 to 254, wherein the neural data processing means comprises:

a first machine learning model for processing the received neural data to identify neural biomarkers; and
a second machine learning model to process the identified neural biomarkers and determine the at least one output signal.

Example 256 is the system of examples 252 to 254, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 257 is the system of examples 252 to 254, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 258 is the system of example 256 or 257, wherein the desired bodily setpoint is calculated within the closed loop controller based on received neural data or received neural biomarkers.

Example 259 is the system of example 257 or 258, wherein the neural control system uses the received data regarding bodily state of the subject from a non-neural sensor to calculate the desired bodily setpoint.

Example 260 is the system of example 256 or 257, wherein the closed loop controller uses the output of a body model to inform the decision of desired change in bodily state.

Example 261 is the system of example 260 wherein the body model is a state space model informed by any combination of neural biomarkers and/or non-neural sensors.

Example 262 is the system of example 260 wherein the body model is a functional model informed by any combination of neural biomarkers or non-neural sensors.

Example 263 is the system of examples 260 to 262 wherein the body model is updated based on received neural or other sensor data describing the bodily state of the subject subsequent to the applied output signal.

Example 264 is the system of example 263 wherein the body model update makes its own estimate of the response to the output signal and uses the comparison of this to the received neural or other sensor data to calculate the update to the body model.

Example 265 is the system of examples 252 to 264, wherein the neural control system further comprises means arranged to receive data regarding a bodily state of the subject from a non-neural sensor;

wherein the at least one machine learning means further processes the received data to determine the at least one output signal.

Example 266 is the system of examples 252 to 265, wherein the neural control system further comprises an output controller arranged to receive the determined output signal and to send the determined output signal to the at least one output device.

Example 267 is the system of example 266, wherein the output controller is arranged to receive selected data regarding received neural data from the input controller;

whereby the input controller and the output controller form a second control loop having a shorter response latency than the first control loop.

Example 268 is the system of example 267, wherein the output controller is arranged to carry out real time modulation of any parameters of the determined output signal sent to the at least one output device based at least in part on the selected data characterizing the received neural data.

Example 269 is the system of example 268, wherein the real time modulation happens in 1 to 100 microseconds.

Example 270 is the system of any one of examples 267 to 269, wherein the output controller is arranged to receive selected data regarding timing of received neural data from the input controller;

wherein the output controller is arranged to control the time at which the determined output signal is sent based at least in part on the selected data regarding timing of received neural data.

Example 271 is the system of any one of examples 267 to 269, wherein the output controller is arranged to receive selected data regarding amplitude of received neural data from the input controller;

wherein the output controller is arranged to control the amplitude and/or frequency of the determined output signal sent to the at least one output device based at least in part on the selected data regarding amplitude of received neural data.

Example 272 is the system of examples 252 to 271, wherein the output signal is a control signal of an end effector device.

Example 273 is the system of examples 252 to 271, wherein the output signal is a neural stimulation signal to be applied to the nervous system of the subject.

Example 274 is the system of examples 252 to 272, wherein the output signal is a control signal for an applied drug treatment.

Example 275 is the system of example 274, wherein the neural control system further comprises an output controller arranged to receive the determined output neural stimulation signal and to send the determined output neural stimulation signal to the at least one output device, and to send data regarding the timing of the determined output neural stimulation signal to the input controller.

Example 276 is the system of example 275, wherein the input controller is arranged to stop sending the received neural data to the at least one machine learning means when the determined output neural stimulation signal is being output.

Example 277 is the system of examples 252 to 276, wherein the neural control system further comprises means for sending the received neural data to an update machine.

Example 278 is the system of examples 252 to 277. wherein the neural control system further comprises means for sending any combination of; the record of calculated neural biomarkers, output signals, recorded neural data, data from other sensors, or data representing bodily state to the update machine.

Example 279 is the system of examples 252 to 278, wherein the neural control system further comprises means for receiving updates of the at least one machine learning model.

Example 280 is the system of example 279, wherein the updates to the at least one machine learning model are calculated based on the received neural data calculated neural biomarkers, output signals, recorded neural data, data from other sensors, or data representing bodily state.

Example 281 is the system of examples 252 to 280, wherein the neural control system further comprises means for receiving updates to the ideal bodily setpoint or calculation of bodily setpoint as a means of updating the applied treatment.

Example 282 is the system of any of examples 260 to 264, wherein the neural control system further comprises means for receiving updates to the body model.

Example 283 is the system of examples 278 to 282 wherein the updates are generated based on data recorded during specific periods of guided activity during rehabilitation or recalibration periods.

Example 284 is the system of example 278 to 282 wherein the update machine is an automated cloud system.

Example 285 is the system of example 278 to 282 wherein the update machine is a manual connection over a local wired or wireless connection.

Example 286 is the system of examples 278 to 282 wherein the updates are automatically calculated by one or more machine learning systems for calculating long term treatment.

Example 287 is the system of examples 278 to 282 wherein the updates are chosen by a treating clinician.

Example 288 is the system of example 252 to 287, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 289 is the system of example 288, wherein the safety module further monitors the performance of the real time input controller and output controller within the real time loop.

Example 290 is the system of examples 288 or 289, wherein the safety module is arranged to act to selectively reduce or stop the function of any element of the neural control system based on monitoring of performance of that element.

Example 291 is the system of examples 288 to 290, wherein the safety module is arranged to selectively control the neural control system to operate in a safety mode.

Example 292 is the system of examples 288 to 291, wherein the neural control system further comprises an auditing module arranged to monitor version control of the neural control system; and

the safety module is arranged to act based on commands from the auditing module.

Example 293 is the system of examples 252 to 292, wherein the neural control system further comprises an auditing module arranged to monitor version control of the neural control system.

Example 294 is the system of example 292 or 293, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements.

Example 295 is the system of examples 292 to 294, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control system of these elements.

Example 296 is the system of any one of examples 292 to 295, wherein the auditing module is arranged to command the safety module to act to reduce or stop the function of the neural control system based on the auditing module detecting at least one of:

Incorrect versioning

Out of date versioning

Unauthorised versioning

Example 297 is the system of examples 252 to 296, in which the at least one neural sensor operates using a first modality and the output signal is a neural stimulation signal to be applied to the nervous system of the subject using a second modality different from the first.

Example 298 is a neural control system comprising:

an input controller arranged to receive neural data regarding neural signals relating to a bodily state from at least one neural sensor;

at least one neural data processing means arranged to process the received neural data to determine at least one output signal required to achieve a desired value of the bodily state; and

means arranged to send the determined output signal to at least one output device;

whereby the neural control system forms a first control loop providing closed loop control of the bodily state; and

wherein the neural control system further comprises an auditing module arranged to monitor version control of the neural control system;

wherein the auditing module is arranged to receive information from the update machine regarding the correct version of the neural control system;

wherein the auditing module is arranged to obtain information regarding the current version of the neural control system;

wherein the auditing module is arranged to command a safety module to act to reduce or stop the function of the neural control system based on the correct and current version of the neural control system.

Example 299 is the system of example 298, wherein the neural control system incorporates the safety module; and

the safety module is arranged for monitoring performance of the neural control system.

Example 300 is the system of example 299, wherein the auditing module is arranged to obtain information regarding the current version of the neural control system of the neural control system from the safety module.

Example 301 is the system of examples 298 to 300, wherein the neural data processing means comprises a single machine learning model for processing the received neural data to directly determine the at least one output signal.

Example 302 is the system of examples 298 to 300, wherein the neural data processing means comprises:

a first machine learning model for processing the received neural data to identify neural biomarkers; and

a second machine learning model to process the identified neural biomarkers and determine the at least one output signal.

Example 303 is the system of examples 298 to 300, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 304 is the system of examples 298 to 300, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 305 is the system of example 303 or 304, wherein the desired bodily setpoint is calculated within the closed loop controller based on received neural data or received neural biomarkers.

Example 306 is the system of example 304 or 305, wherein the neural control system uses the received data regarding bodily state of the subject from a non-neural sensor to calculate the desired bodily setpoint.

Example 307 is the system of example 303 or 304, wherein the closed loop controller uses the output of a body model to inform the decision of desired change in bodily state.

Example 308 is the system of example 307 wherein the body model is a state space model informed by any combination of neural biomarkers and/or non-neural sensors.

Example 309 is the system of example 307 wherein the body model is a functional model informed by any combination of neural biomarkers or non-neural sensors.

Example 310 is the system of examples 307 to 309 wherein the body model is updated based on received neural or other sensor data describing the bodily state of the subject subsequent to the applied output signal.

Example 311 is the system of example 310 wherein the body model update makes its own estimate of the response to the output signal and uses the comparison of this to the received neural or other sensor data to calculate the update to the body model.

Example 312 is the system of examples 298 to 310, wherein the neural control system further comprises means arranged to receive data regarding a bodily state of the subject from a non-neural sensor;

wherein the at least one machine learning means further processes the received data to determine the at least one output signal.

Example 313 is the system of examples 298 to 312, wherein the neural control system further comprises an output controller arranged to receive the determined output signal and to send the determined output signal to the at least one output device.

Example 314 is the system of example 313, wherein the output controller is arranged to receive selected data regarding received neural data from the input controller;

whereby the input controller and the output controller form a second control loop having a shorter response latency than the first control loop.

Example 315 is the system of example 314, wherein the output controller is arranged to carry out real time modulation of any parameters of the determined output signal sent to the at least one output device based at least in part on the selected data characterizing the received neural data.

Example 316 is the system of example 315, wherein the real time modulation happens in 1 to 100 microseconds.

Example 317 is the system of any one of examples 313 to 316, wherein the output controller is arranged to receive selected data regarding timing of received neural data from the input controller;

wherein the output controller is arranged to control the time at which the determined output signal is sent based at least in part on the selected data regarding timing of received neural data.

Example 318 is the system of any one of examples 313 to 316, wherein the output controller is arranged to receive selected data regarding amplitude of received neural data from the input controller;

wherein the output controller is arranged to control the amplitude and/or frequency of the determined output signal sent to the at least one output device based at least in part on the selected data regarding amplitude of received neural data.

Example 319 is the system of examples 298 to 318, wherein the output signal is a control signal of an end effector device.

Example 320 is the system of examples 298 to 319, wherein the output signal is a neural stimulation signal to be applied to the nervous system of the subject.

Example 321 is the system of examples 298 to 320, wherein the output signal is a control signal for an applied drug treatment.

Example 322 is the system of example 321, wherein the neural control system further comprises an output controller arranged to receive the determined output neural stimulation signal and to send the determined output neural stimulation signal to the at least one output device, and to send data regarding the timing of the determined output neural stimulation signal to the input controller.

Example 323 is the system of example 322, wherein the input controller is arranged to stop sending the received neural data to the at least one machine learning means when the determined output neural stimulation signal is being output.

Example 324 is the system of examples 298 to 323, wherein the neural control system further comprises means for sending the received neural data to an update machine.

Example 325 is the system of examples 298 to 324, wherein the neural control system further comprises means for sending any combination of; the record of calculated neural biomarkers, output signals, recorded neural data, data from other sensors, or data representing bodily state to the update machine.

Example 326 is the system of examples 298 to 325, wherein the neural control system further comprises means for receiving updates of the at least one machine learning model.

Example 327 is the system of example 326, wherein the updates to the at least one machine learning model are calculated based on the received neural data calculated neural biomarkers, output signals, recorded neural data, data from other sensors, or data representing bodily state.

Example 328 is the system of examples 298 to 327, wherein the neural control system further comprises means for receiving updates to the ideal bodily setpoint or calculation of bodily setpoint as a means of updating the applied treatment.

Example 329 is the system of any of examples 305 to 311, wherein the neural control system further comprises means for receiving updates to the body model.

Example 330 is the system of examples 325 to 329 wherein the updates are generated based on data recorded during specific periods of guided activity during rehabilitation or recalibration periods.

Example 331 is the system of example 325 to 329 wherein the update machine is an automated cloud system.

Example 332 is the system of example 325 to 329 wherein the update machine is a manual connection over a local wired or wireless connection.

Example 333 is the system of examples 325 to 329 wherein the updates are automatically calculated by one or more machine learning systems for calculating long term treatment.

Example 334 is the system of examples 325 to 329 wherein the updates are chosen by a treating clinician.

Example 335 is the system of example 299 or example 300, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 336 is the system of example 335, wherein the safety module further monitors the performance of the real time input controller and output controller within the real time loop.

Example 337 is the system of examples 335 or 336, wherein the safety module is arranged to act to selectively reduce or stop the function of any element of the neural control system based on monitoring of performance of that element.

Example 338 is the system of examples 335 to 337, wherein the safety module is arranged to selectively control the neural control system to operate in a safety mode.

Example 339 is the system of examples 335 to 338, wherein the safety module is arranged to act based on commands from the auditing module.

Example 340 is the system of example 298 to 339, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements.

Example 341 is the system of example 298 to 340, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control system of these elements.

Example 342 is the system of examples 298 to 341, wherein the auditing module is arranged to command the safety module to act to reduce or stop the function of the neural control system based on the auditing module detecting at least one of:

Incorrect versioning

Out of date versioning

Unauthorised versioning.

Example 343 is the system of example of examples 298 to 342, in which the at least one neural sensor operates using a first modality and the output signal is a neural stimulation signal to be applied to the nervous system of the subject using a second modality different from the first.

Example 344 is a neural control system comprising:

an input controller arranged to receive neural data regarding neural signals relating to a bodily state from at least one neural sensor;

at least one neural data processing means arranged to process the received neural data to determine at least one output signal required to achieve a desired value of the bodily state; and

means arranged to send the determined output signal to at least one output device;
whereby the neural control system forms a first control loop providing closed loop control of the bodily state; and
wherein the desired value of bodily state is achieved by a bodily setpoint set in advance as a treatment.

Example 345 is the system of example 344, wherein the neural data processing means comprises a single machine learning model for processing the received neural data to directly determine the at least one output signal.

Example 346 is the system of example 344, wherein the neural data processing means comprises:

a first machine learning model for processing the received neural data to identify neural biomarkers; and
a second machine learning model to process the identified neural biomarkers and determine the at least one output signal.

Example 347 is the system of example 344, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 348 is the system of example 344, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 349 is the system of example 347 or 348 wherein the desired bodily setpoint is calculated within the closed loop controller based on received neural data or received neural biomarkers.

Example 350 is the system of example 348 or 349, wherein the neural control system uses the received data regarding bodily state of the subject from a non-neural sensor to calculate the desired bodily setpoint.

Example 351 is the system of example 347 or 348, wherein the closed loop controller uses the output of a body model to inform the decision of desired change in bodily state.

Example 352 is the system of example 351 wherein the body model is a state space model informed by any combination of neural biomarkers and/or non-neural sensors.

Example 353 is the system of example 351 wherein the body model is a functional model informed by any combination of neural biomarkers or non-neural sensors.

Example 354 is the system of examples 351 to 353 wherein the body model is updated based on received neural or other sensor data describing the bodily state of the subject subsequent to the applied output signal.

Example 355 is the system of example 354 wherein the body model update makes its own estimate of the response to the output signal and uses the comparison of this to the received neural or other sensor data to calculate the update to the body model.

Example 356 is the system of examples 344 to 355, wherein the neural control system further comprises means arranged to receive data regarding a bodily state of the subject from a non-neural sensor;

wherein the at least one machine learning means further processes the received data to determine the at least one output signal.

Example 357 is the system of examples 345 to 356, wherein the neural control system further comprises an output controller arranged to receive the determined output signal and to send the determined output signal to the at least one output device.

Example 358 is the system of example 357, wherein the output controller is arranged to receive selected data regarding received neural data from the input controller;

whereby the input controller and the output controller form a second control loop having a shorter response latency than the first control loop.

Example 359 is the system of example 358, wherein the output controller is arranged to carry out real time modulation of any parameters of the determined output signal sent to the at least one output device based at least in part on the selected data characterizing the received neural data.

Example 360 is the system of example 359, wherein the real time modulation happens in 1 to 100 microseconds.

Example 361 is the system of any one of examples 358 to 360, wherein the output controller is arranged to receive selected data regarding timing of received neural data from the input controller;

wherein the output controller is arranged to control the time at which the determined output signal is sent based at least in part on the selected data regarding timing of received neural data.

Example 362 is the system of any one of examples 358 to 360, wherein the output controller is arranged to receive selected data regarding amplitude of received neural data from the input controller;

wherein the output controller is arranged to control the amplitude and/or frequency of the determined output signal sent to the at least one output device based at least in part on the selected data regarding amplitude of received neural data.

Example 363 is the system of examples 344 to 362, wherein the output signal is a control signal of an end effector device.

Example 364 is the system of examples 344 to 363, wherein the output signal is a neural stimulation signal to be applied to the nervous system of the subject.

Example 365 is the system of examples 344 to 364, wherein the output signal is a control signal for an applied drug treatment.

Example 366 is the system of example 365, wherein the neural control system further comprises an output controller arranged to receive the determined output neural stimulation signal and to send the determined output neural stimulation signal to the at least one output device, and to send data regarding the timing of the determined output neural stimulation signal to the input controller.

Example 367 is the system of example 366, wherein the input controller is arranged to stop sending the received neural data to the at least one machine learning means when the determined output neural stimulation signal is being output.

Example 368 is the system of examples 344 to 367, wherein the neural control system further comprises means for sending the received neural data to an update machine.

Example 369 is the system of examples 344 to 368. wherein the neural control system further comprises means for sending any combination of; the record of calculated neural biomarkers, output signals, recorded neural data, data from other sensors, or data representing bodily state to the update machine.

Example 370 is the system of examples 344 to 369, wherein the neural control system further comprises means for receiving updates of the at least one machine learning model.

Example 371 is the system of example 370, wherein the updates to the at least one machine learning model are calculated based on the received neural data calculated neural biomarkers, output signals, recorded neural data, data from other sensors, or data representing bodily state.

Example 372 is the system of examples 344 to 371, wherein the neural control system further comprises means for receiving updates to the ideal bodily setpoint or calculation of bodily setpoint as a means of updating the applied treatment.

Example 373 is the system of any of examples 351 to 355, wherein the neural control system further comprises means for receiving updates to the body model.

Example 374 is the system of examples 371 to 373 wherein the updates are generated based on data recorded during specific periods of guided activity during rehabilitation or recalibration periods.

Example 375 is the system of example 369 to 373 wherein the update machine is an automated cloud system.

Example 376 is the system of example 369 to 373 wherein the update machine is a manual connection over a local wired or wireless connection.

Example 377 is the system of examples 369 to 373 wherein the updates are automatically calculated by one or more machine learning systems for calculating long term treatment.

Example 378 is the system of examples 369 to 373 wherein the updates are chosen by a treating clinician.

Example 379 is the system of examples 344 to 378, wherein the neural control system incorporates a safety module for monitoring performance of the neural control system.

Example 380 is the system of example 379, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 381 is the system of example 379 or 380, wherein the safety module further monitors the performance of the real time input controller and output controller within the real time loop.

Example 382 is the system of examples 379 to 381, wherein the safety module is arranged to act to selectively reduce or stop the function of any element of the neural control system based on monitoring of performance of that element.

Example 383 is the system of examples 379 to 382, wherein the safety module is arranged to selectively control the neural control system to operate in a safety mode.

Example 384 is the system of examples 379 to 383, wherein the neural control system further comprises an auditing module arranged to monitor version control of the neural control system; and

the safety module is arranged to act based on commands from the auditing module.

Example 385 is the system of examples 344 to 384, wherein the neural control system further comprises an auditing module arranged to monitor version control of the neural control system.

Example 386 is the system of example 384 or 385, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements.

Example 387 is the system of example 385 or 386, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control system of these elements.

Example 388 is the system of any one of examples 385 to 386, wherein the auditing module is arranged to command the safety module to act to reduce or stop the function of the neural control system based on the auditing module detecting at least one of:

Incorrect versioning

Out of date versioning

Unauthorised versioning

Example 389 is the system of examples 344 to 388 in which the at least one neural sensor operates using a first modality and the output signal is a neural stimulation signal to be applied to the nervous system of the subject using a second modality different from the first.

Example 390 is a neural control system comprising:

an input controller arranged to receive neural data regarding neural signals relating to a bodily state from at least one neural sensor;

at least one neural data processing means arranged to process the received neural data to determine at least one output signal required to achieve a desired value of the bodily state; and

means arranged to send the determined output signal to at least one output device;
whereby the neural control system forms a first control loop providing closed loop control of the bodily state;
wherein the desired value of bodily state is achieved by a bodily setpoint; and
wherein the setpoint is calculated within the closed loop controller based on received sensor data.

Example 391 is the system of example 390, wherein the neural data processing means comprises a single machine learning model for processing the received neural data to directly determine the at least one output signal.

Example 392 is the system of example 390, wherein the neural data processing means comprises:

a first machine learning model for processing the received neural data to identify neural biomarkers; and
a second machine learning model to process the identified neural biomarkers and determine the at least one output signal.

Example 393 is the system of example 390, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 394 is the system of example 390, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 395 is the system of example 393 or 394, wherein the desired bodily setpoint is calculated within the closed loop controller based on received neural data or received neural biomarkers.

Example 396 is the system of example 394 or 395, wherein the neural control system uses the received data regarding bodily state of the subject from a non-neural sensor to calculate the desired bodily setpoint.

Example 397 is the system of example 393 or 394, wherein the closed loop controller uses the output of a body model to inform the decision of desired change in bodily state.

Example 398 is the system of example 397 wherein the body model is a state space model informed by any combination of neural biomarkers and/or non-neural sensors.

Example 399 is the system of example 397 wherein the body model is a functional model informed by any combination of neural biomarkers or non-neural sensors.

Example 400 is the system of examples 397 to 399, wherein the body model is updated based on received neural or other sensor data describing the bodily state of the subject subsequent to the applied output signal.

Example 401 is the system of example 400 wherein the body model update makes its own estimate of the response to the output signal and uses the comparison of this to the received neural or other sensor data to calculate the update to the body model.

Example 402 is the system of examples 390 to 401, wherein the neural control system further comprises means arranged to receive data regarding a bodily state of the subject from a non-neural sensor;

wherein the at least one machine learning means further processes the received data to determine the at least one output signal.

Example 403 is the system of examples 390 to 402, wherein the neural control system further comprises an output controller arranged to receive the determined output signal and to send the determined output signal to the at least one output device.

Example 404 is the system of example 403, wherein the output controller is arranged to receive selected data regarding received neural data from the input controller;

whereby the input controller and the output controller form a second control loop having a shorter response latency than the first control loop.

Example 405 is the system of example 404, wherein the output controller is arranged to carry out real time modulation of any parameters of the determined output signal sent to the at least one output device based at least in part on the selected data characterizing the received neural data.

Example 406 is the system of example 405, wherein the real time modulation happens in 1 to 100 microseconds.

Example 407 is the system of any one of examples 404 to 406, wherein the output controller is arranged to receive selected data regarding timing of received neural data from the input controller;

wherein the output controller is arranged to control the time at which the determined output signal is sent based at least in part on the selected data regarding timing of received neural data.

Example 408 is the system of any one of examples 404 to 407, wherein the output controller is arranged to receive selected data regarding amplitude of received neural data from the input controller;

wherein the output controller is arranged to control the amplitude and/or frequency of the determined output signal sent to the at least one output device based at least in part on the selected data regarding amplitude of received neural data.

Example 409 is the system of examples 390 to 408, wherein the output signal is a control signal of an end effector device.

Example 410 is the system of examples 390 to 409, wherein the output signal is a neural stimulation signal to be applied to the nervous system of the subject.

Example 411 is the system of examples 390 to 410, wherein the output signal is a control signal for an applied drug treatment.

Example 412 is the system of example 411, wherein the neural control system further comprises an output controller arranged to receive the determined output neural stimulation signal and to send the determined output neural stimulation signal to the at least one output device, and to send data regarding the timing of the determined output neural stimulation signal to the input controller.

Example 413 is the system of example 412, wherein the input controller is arranged to stop sending the received neural data to the at least one machine learning means when the determined output neural stimulation signal is being output.

Example 414 is the system of examples 390 to 413, wherein the neural control system further comprises means for sending the received neural data to an update machine.

Example 415 is the system of examples 390 to 414. wherein the neural control system further comprises means for sending any combination of; the record of calculated neural biomarkers, output signals, recorded neural data, data from other sensors, or data representing bodily state to the update machine.

Example 416 is the system of examples 390 to 415, wherein the neural control system further comprises means for receiving updates of the at least one machine learning model.

Example 417 is the system of example 416, wherein the updates to the at least one machine learning model are calculated based on the received neural data calculated neural biomarkers, output signals, recorded neural data, data from other sensors, or data representing bodily state.

Example 418 is the system of examples 390 to 417, wherein the neural control system further comprises means for receiving updates to the ideal bodily setpoint or calculation of bodily setpoint as a means of updating the applied treatment.

Example 419 is the system of any of examples 397 to 401, wherein the neural control system further comprises means for receiving updates to the body model.

Example 420 is the system of examples 415 to 419 wherein the updates are generated based on data recorded during specific periods of guided activity during rehabilitation or recalibration periods.

Example 421 is the system of example 415 to 419 wherein the update machine is an automated cloud system.

Example 422 is the system of example 415 to 419 wherein the update machine is a manual connection over a local wired or wireless connection.

Example 423 is the system of examples 415 to 419 wherein the updates are automatically calculated by one or more machine learning systems for calculating long term treatment.

Example 424 is the system of examples 415 to 419 wherein the updates are chosen by a treating clinician.

Example 425 is the system of examples 390 to 424, wherein the neural control system incorporates a safety module for monitoring performance of the neural control system.

Example 426 is the system of example 425, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 427 is the system of example 425 or 426, wherein the safety module further monitors the performance of the real time input controller and output controller within the real time loop.

Example 428 is the system of examples 425 to 427, wherein the safety module is arranged to act to selectively reduce or stop the function of any element of the neural control system based on monitoring of performance of that element.

Example 429 is the system of examples 425 to 428, wherein the safety module is arranged to selectively control the neural control system to operate in a safety mode.

Example 430 is the system of examples 425 to 429, wherein the neural control system further comprises an auditing module arranged to monitor version control of the neural control system; and

the safety module is arranged to act based on commands from the auditing module.

Example 431 is the system of examples 390 to 429, wherein the neural control system further comprises an auditing module arranged to monitor version control of the neural control system.

Example 432 is the system of example 430 or 431, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements.

Example 433 is the system of example 431 or 432, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control system of these elements.

Example 434 is the system of any one of examples 431 to 433, wherein the auditing module is arranged to command the safety module to act to reduce or stop the function of the neural control system based on the auditing module detecting at least one of:

Incorrect versioning

Out of date versioning

Unauthorised versioning

Example 435 is the system of examples 390 to 434 in which the at least one neural sensor operates using a first modality and the output signal is a neural stimulation signal to be applied to the nervous system of the subject using a second modality different from the first.

Example 436 is a neural control system comprising:

an input controller arranged to receive neural data regarding neural signals relating to a bodily state from at least one neural sensor;

at least one neural data processing means arranged to process the received neural data to determine at least one output signal required to achieve a desired value of the bodily state; and

means arranged to send the determined output signal to at least one output device;
whereby the neural control system forms a first control loop providing closed loop control of the bodily state; and
wherein the closed loop controller uses the output of a body model to inform the decision of desired change in bodily state;
wherein the current state of the body relative to the body model is informed by any combination of neural biomarkers or non-neural sensors.

Example 437 is the system of example 436, wherein the neural data processing means comprises a single machine learning model for processing the received neural data to directly determine the at least one output signal.

Example 438 is the system of example 436, wherein the neural data processing means comprises:

a first machine learning model for processing the received neural data to identify neural biomarkers; and
a second machine learning model to process the identified neural biomarkers and determine the at least one output signal.

Example 439 is the system of example 436, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 440 is the system of example 436, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 441 is the system of example 439 or 440, wherein the desired bodily setpoint is calculated within the closed loop controller based on received neural data or received neural biomarkers.

Example 442 is the system of example 440 or 441, wherein the neural control system uses the received data regarding bodily state of the subject from a non-neural sensor to calculate the desired bodily setpoint.

Example 443 is the system of examples 436 to 442, wherein the body model is a state space model informed by any combination of neural biomarkers and/or non-neural sensors.

Example 444 is the system of examples 436 to 442, wherein the body model is a functional model informed by any combination of neural biomarkers or non-neural sensors.

Example 445 is the system of examples 436 to 444, wherein the body model is updated based on received neural or other sensor data describing the bodily state of the subject subsequent to the applied output signal.

Example 446 is the system of example 445 wherein the body model update makes its own estimate of the response to the output signal and uses the comparison of this to the received neural or other sensor data to calculate the update to the body model.

Example 447 is the system of examples 436 to 446, wherein the neural control system further comprises means arranged to receive data regarding a bodily state of the subject from a non-neural sensor;

wherein the at least one machine learning means further processes the received data to determine the at least one output signal.

Example 448 is the system of examples 436 to 447, wherein the neural control system further comprises an output controller arranged to receive the determined output signal and to send the determined output signal to the at least one output device.

Example 449 is the system of example 448, wherein the output controller is arranged to receive selected data regarding received neural data from the input controller;

whereby the input controller and the output controller form a second control loop having a shorter response latency than the first control loop.

Example 450 is the system of example 449, wherein the output controller is arranged to carry out real time modulation of any parameters of the determined output signal sent to the at least one output device based at least in part on the selected data characterizing the received neural data.

Example 451 is the system of example 450, wherein the real time modulation happens in 1 to 100 microseconds.

Example 452 is the system of any one of examples 449 to 451, wherein the output controller is arranged to receive selected data regarding timing of received neural data from the input controller;

wherein the output controller is arranged to control the time at which the determined output signal is sent based at least in part on the selected data regarding timing of received neural data.

Example 453 is the system of any one of examples 449 to 452, wherein the output controller is arranged to receive selected data regarding amplitude of received neural data from the input controller;

wherein the output controller is arranged to control the amplitude and/or frequency of the determined output signal sent to the at least one output device based at least in part on the selected data regarding amplitude of received neural data.

Example 454 is the system of examples 436 to 453, wherein the output signal is a control signal of an end effector device.

Example 455 is the system of examples 436 to 454, wherein the output signal is a neural stimulation signal to be applied to the nervous system of the subject.

Example 456 is the system of examples 436 to 454, wherein the output signal is a control signal for an applied drug treatment.

Example 457 is the system of example 456, wherein the neural control system further comprises an output controller arranged to receive the determined output neural stimulation signal and to send the determined output neural stimulation signal to the at least one output device, and to send data regarding the timing of the determined output neural stimulation signal to the input controller.

Example 458 is the system of example 457, wherein the input controller is arranged to stop sending the received neural data to the at least one machine learning means when the determined output neural stimulation signal is being output.

Example 459 is the system of examples 436 to 458, wherein the neural control system further comprises means for sending the received neural data to an update machine.

Example 460 is the system of examples 436 to 459. wherein the neural control system further comprises means for sending any combination of; the record of calculated neural biomarkers, output signals, recorded neural data, data from other sensors, or data representing bodily state to the update machine.

Example 461 is the system of examples 436 to 460, wherein the neural control system further comprises means for receiving updates of the at least one machine learning model.

Example 462 is the system of example 461, wherein the updates to the at least one machine learning model are calculated based on the received neural data calculated neural biomarkers, output signals, recorded neural data, data from other sensors, or data representing bodily state.

Example 463 is the system of examples 436 to 462, wherein the neural control system further comprises means for receiving updates to the ideal bodily setpoint or calculation of bodily setpoint as a means of updating the applied treatment.

Example 464 is the system of any of examples 436 to 463, wherein the neural control system further comprises means for receiving updates to the body model.

Example 465 is the system of examples 460 to 464 wherein the updates are generated based on data recorded during specific periods of guided activity during rehabilitation or recalibration periods.

Example 466 is the system of example 460 to 464 wherein the update machine is an automated cloud system.

Example 467 is the system of example 460 to 464 wherein the update machine is a manual connection over a local wired or wireless connection.

Example 468 is the system of examples 460 to 464 wherein the updates are automatically calculated by one or more machine learning systems for calculating long term treatment.

Example 469 is the system of examples 460 to 464 wherein the updates are chosen by a treating clinician.

Example 470 is the system of examples 436 to 469, wherein the neural control system incorporates a safety module for monitoring performance of the neural control system.

Example 471 is the system of example 470, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 472 is the system of example 470 or 471, wherein the safety module further monitors the performance of the real time input controller and output controller within the real time loop.

Example 473 is the system of examples 470 to 472, wherein the safety module is arranged to act to selectively reduce or stop the function of any element of the neural control system based on monitoring of performance of that element.

Example 474 is the system of examples 470 to 473, wherein the safety module is arranged to selectively control the neural control system to operate in a safety mode.

Example 475 is the system of examples 470 to 474, wherein the neural control system further comprises an auditing module arranged to monitor version control of the neural control system; and

the safety module is arranged to act based on commands from the auditing module.

Example 476 is the system of examples 436 to 475, wherein the neural control system further comprises an auditing module arranged to monitor version control of the neural control system.

Example 477 is the system of example 475 or 476, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements.

Example 478 is the system of example 476 or 477, wherein the neural control system comprises one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control system of these elements.

Example 479 is the system of any one of examples 476 to 478, wherein the auditing module is arranged to command the safety module to act to reduce or stop the function of the neural control system based on the auditing module detecting at least one of:

Incorrect versioning

Out of date versioning

Unauthorised versioning

Example 480 is the system of examples 436 to 479 in which the at least one neural sensor operates using a first modality and the output signal is a neural stimulation signal to be applied to the nervous system of the subject using a second modality different from the first.

Example 481 is a neural control method comprising:

using an input controller to receive neural data regarding neural signals relating to a bodily state of a subject from at least one neural sensor;
using at least one machine learning model to process the received neural data to determine at least one output signal required to achieve a desired value of the bodily state; and
sending the determined output signal to at least one output device;
wherein the method forms a first control loop providing closed loop control of the bodily state.

Example 482 is the method of example 481, wherein the at least one machine learning model comprises using a single machine learning model for processing the received neural data to directly determine the at least one output signal.

Example 483 is the method of example 481, wherein the at least one machine learning model comprises:

a first machine learning model for processing the received neural data to identify neural biomarkers; and
a second machine learning model to process the identified neural biomarkers and determine the at least one output signal.

Example 484 is the method of example 481, wherein the at least one machine learning model comprises:

a first machine learning model for processing the received neural data to identify neural biomarkers; and

a closed loop controller receiving the neural biomarkers describing bodily state alongside the desired bodily setpoint to determine the output signal.

Example 485 is the method of example 481, wherein the at least one machine learning model comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 486 is the method of example 484 or 485, wherein the desired bodily setpoint is calculated within the closed loop controller based on received neural data or received neural biomarkers.

Example 487 is the method of example 485 or 486, further comprising using the received data regarding bodily state of the subject from a non-neural sensor to calculate the desired bodily setpoint.

Example 488 is the method of example 484 or 485, wherein the closed loop controller uses the output of a body model to inform the decision of desired change in bodily state.

Example 489 is the method of example 488 wherein the body model is a state space model informed by any combination of neural biomarkers and/or non-neural sensors.

Example 490 is the method of example 488 wherein the body model is a functional model informed by any combination of neural biomarkers or non-neural sensors.

Example 491 is the method of example 488, 489, or 490 wherein the body model is updated based on received neural or other sensor data describing the bodily state of the subject subsequent to the applied output signal.

Example 492 is the method of example 491 wherein the body model update makes its own estimate of the response to the output signal and uses the comparison of this to the received neural or other sensor data to calculate the update to the body model.

Example 493 is the method of any one of examples 481 to 492, wherein the neural control method further comprises receiving data regarding a bodily state of the subject from a non-neural sensor;

wherein the at least one machine learning means further processes the received data to determine the at least one output signal.

Example 494 is the method of any one of examples 481 to 492, wherein the neural control method further comprises using an output controller to receive the determined output signal and to send the determined output signal to the at least one output device.

Example 495 is the method of example 494, wherein the output controller receives selected data regarding received neural data from the input controller;

whereby the input controller and the output controller form a second control loop having a shorter response latency than the first control loop.

Example 496 is the method of example 495, wherein the output controller carries out real time modulation of any parameters of the determined output signal sent to the at least one output device based at least in part on the selected data characterizing the received neural data.

Example 497 is the method of example 496, wherein the real time modulation happens in 1 to 100 microseconds.

Example 498 is the method of any one of examples 495 to 497, wherein the output controller receives selected data regarding timing of received neural data from the input controller;

wherein the output controller controls the time at which the determined output signal is sent based at least in part on the selected data regarding timing of received neural data.

Example 499 is the method of any one of examples 495 to 497, wherein the output controller receives selected data regarding amplitude of received neural data from the input controller;

wherein the output controller controls the amplitude and/or frequency of the determined output signal sent to the at least one output device based at least in part on the selected data regarding amplitude of received neural data.

Example 500 is the method of any one of examples 481 to 499, wherein the output signal is a control signal of an end effector device.

Example 501 is the method of any one of examples 481 to 500, wherein the output signal is a neural stimulation signal to be applied to the nervous system of the subject.

Example 502 is the method of any one of examples 481 to 501, wherein the output signal is a control signal for an applied drug treatment.

Example 503 is the method of example 501, wherein the neural control method further comprises using an output controller to receive the determined output neural stimulation signal and to send the determined output neural stimulation signal to the at least one output device, and to send data regarding the timing of the determined output neural stimulation signal to the input controller.

Example 504 is the method of example 503, wherein the input controller stops sending the received neural data to the at least one machine learning means when the determined output neural stimulation signal is being output.

Example 505 is the method of any one of examples 481 to 504, wherein the neural control method further comprises sending the received neural data to an update machine.

Example 506 is the method of any one of examples 481 to 505, wherein the neural control method further comprises sending any combination of; the record of calculated neural biomarkers, output signals, recorded neural data, data from other sensors, or data representing bodily state to the update machine.

Example 507 is the method of any one of examples 481 to 506, wherein the neural control method further comprises receiving updates of the at least one machine learning model.

Example 508 is the method of example 507, wherein the updates to the at least one machine learning model are calculated based on the received neural data, calculated neural biomarkers, output signals, recorded neural data, data from other sensors, or data representing bodily state.

Example 509 is the method of any one of examples 481 to 508, wherein the neural control method further comprises receiving updates to the ideal bodily setpoint or calculation of bodily setpoint as a means of updating the applied treatment.

Example 510 is the method of any of examples 488 to 492, wherein the neural control method further comprises receiving updates to the body model.

Example 511 is the method of examples 506 to 510 wherein the updates are generated based on data recorded during specific periods of guided activity during rehabilitation or recalibration periods.

Example 512 is the method of example 506 to 510 wherein the update machine is an automated cloud method.

Example 513 is the method of example 506 to 510 wherein the update machine is a manual connection over a local wired or wireless connection.

Example 514 is the method of examples 506 to 510 wherein the updates are automatically calculated by one or more machine learning methods for calculating long term treatment.

Example 515 is the method of examples 506 to 510 wherein the updates are chosen by a treating clinician.

Example 516 is the method of any one of example 481 to 515, wherein the neural control method incorporates using a safety module for monitoring performance of the neural control method.

Example 517 is the method of example 516, wherein the neural control method comprises using one or more elements from the group of:

Machine learning data processor;

Control module;

Real time input controller;

Real time output controller;

Stimulation library selector;

Machine learning output processor;

Bodily setpoint calculator; and

Body model;

wherein the performance of these elements is monitored by the safety module.

Example 518 is the method of example 516 or 517, wherein the safety module further monitors the performance of the real time input controller and output controller within the real time loop.

Example 519 is the method of examples 516 to 518, wherein the safety module acts to selectively reduce or stop the function of any element of the neural control method based on monitoring of performance of that element.

Example 520 is the method of examples 516 to 519, wherein the safety module selectively controls the neural control method to operate in a safety mode.

Example 521 is the method of examples 516 to 520, wherein the neural control method further comprises using an auditing module to monitor version control of the neural control method; and

the safety module acts based on commands from the auditing module.

Example 522 is the method of any one of examples 481 to 521, wherein the neural control method further comprises using an auditing module to monitor version control of the neural control method.

Example 523 is the method of example 521 or 522, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and
Body model;
   wherein the auditing module is arranged to receive information from the update machine regarding the correct version of these elements.

Example 524 is the method of example 522 or 523, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control method of these elements.

Example 525 is the method of any one of examples 522 to 524, wherein the auditing module is arranged to command the safety module to act to reduce or stop the function of the neural control method based on the auditing module detecting at least one of:

Incorrect versioning;

Out of date versioning;

Unauthorised versioning.

Example 526 is the method of any one of examples 481 to 524 in which the at least one neural sensor operates using a first modality and the output signal is a neural stimulation signal to be applied to the nervous system of the subject using a second modality different from the first.

Example 527 is a neural control method comprising:

using an input controller to receive neural data regarding neural signals relating to a bodily state from at least one neural sensor;

using at least one machine learning model for processing the received neural data to identify neural biomarkers; and

sending the identified neural biomarkers to at least one output device for performing operations;

wherein the neural control method forms a first control loop providing closed loop control of the bodily state.

Example 528 is the method of example 527, wherein the neural biomarkers are learnt over a population of patients to be stationary representations of neural population activity.

Example 529 is the method of example 527 or 528, wherein the neural biomarkers are learnt over a population of patients to be stationary representations of neural population activity.

Example 530 is the method of any of examples 527 to 529, wherein the neural control method further comprises receiving updates of the at least one machine learning model.

Example 531 is the method of example 530, wherein the updates to the at least one machine learning model are calculated based on the received neural data calculated neural biomarkers, output signals, recorded neural data, data from other sensors, or data representing bodily state.

Example 532 is the method of any of examples 527 to 531, wherein the neural control method further comprises receiving updates to the ideal bodily setpoint or calculation of bodily setpoint as a means of updating the applied treatment.

Example 533 is the method of any of examples 527 to 532, wherein the neural control method further comprises receiving updates to the body model.

Example 534 is the method of examples 527 to 533 wherein the updates are generated based on data recorded during specific periods of guided activity during rehabilitation or recalibration periods.

Example 535 is the method of example 527 to 533 wherein the updates are received from an update machine which is an automated cloud method.

Example 536 is the method of example 527 to 533 wherein the update machine is a manual connection over a local wired or wireless connection.

Example 537 is the method of examples 527 to 533 wherein the updates are automatically calculated by one or more machine learning methods for calculating long term treatment.

Example 538 is the method of examples 527 to 533 wherein the updates are chosen by a treating clinician.

Example 539 is the method of any one of examples 527 to 538, wherein the neural control method comprises using a safety module for monitoring performance of the neural control method.

Example 540 is the method of example 539, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 541 is the method of example 539 or 540 wherein the safety module is arranged to act to selectively reduce or stop the function of any element of the neural control method based on monitoring of performance of that element.

Example 542 is the method of examples 539 to 541, wherein the safety module is arranged to selectively control the neural control method to operate in a safety mode.

Example 543 is the method of examples 539 to 542, wherein the neural control method further comprises using an auditing module monitor version control of the neural control method; and

the safety module is arranged to act based on commands from an auditing module.

Example 544 is the method of any one of examples 537 to 543, wherein the neural control method further comprises using an auditing module to monitor version control of the neural control method.

Example 545 is the method of example 543 or 544, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements

Example 546 is the method of example 544 or 545, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control method of these elements.

Example 547 is the method of any one of examples 542 to 546, wherein the auditing module is arranged to command the safety module to act to reduce or stop the function of the neural control method based on the auditing module detecting at least one of:

Incorrect versioning;

Out of date versioning;

Unauthorised versioning.

Example 548 is the method of example of any of examples 527 to 547, wherein the at least one neural sensor operates using a first modality and the output signal is a neural stimulation signal to be applied to the nervous system of the subject using a second modality different from the first.

Example 549 is a neural control method comprising:

using an input controller to receive neural data regarding neural signals relating to a bodily state in nerves of a subject from at least one neural sensor;

using at least one neural data processing means to process the received neural data to determine at least one output signal required to achieve a desired value of the bodily state; and

using an output controller to receive the determined output signal and to send the determined output signal to the at least one output device;

whereby the neural control system forms a first control loop providing closed loop control of the bodily state;
wherein the output controller is further used to receive selected data regarding timing of received neural data from the input controller; and
wherein the output controller is used to control the time at which the determined output signal is sent to the at least one output device based on the selected data regarding timing of received neural data.

Example 550 is the method of example 549, wherein the neural data processing means comprises a single machine learning model for processing the received neural data to directly determine the at least one output signal.

Example 551 is the method of example 549, wherein the neural data processing means comprises:

a first machine learning model for processing the received neural data to identify neural biomarkers; and
a second machine learning model to process the identified neural biomarkers and determine the at least one output signal.

Example 552 is the method of example 549, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 553 is the method of example 549, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 554 is the method of example 552 or 553, wherein the desired bodily setpoint is calculated within the closed loop controller based on received neural data or received neural biomarkers.

Example 555 is the method of example 553 or 554, wherein the neural control method uses the received data regarding bodily state of the subject from a non-neural sensor to calculate the desired bodily setpoint.

Example 556 is the method of example 552 or 553, wherein the closed loop controller uses the output of a body model to inform the decision of desired change in bodily state.

Example 557 is the method of example 556 wherein the body model is a state space model informed by any combination of neural biomarkers and/or non-neural sensors.

Example 558 is the method of example 556 wherein the body model is a functional model informed by any combination of neural biomarkers or non-neural sensors.

Example 559 is the method of examples 556 to 558 wherein the body model is updated based on received neural or other sensor data describing the bodily state of the subject subsequent to the applied output signal.

Example 560 is the method of example 559 wherein the body model update makes its own estimate of the response to the output signal and uses the comparison of this to the received neural or other sensor data to calculate the update to the body model.

Example 561 is the method of any of examples 549 to 560, wherein the neural control method further comprises receiving data regarding a bodily state of the subject from a non-neural sensor;

wherein the at least one machine learning means further processes the received data to determine the at least one output signal.

Example 562 is the method of any of examples 549 to 561, wherein the output controller receives selected data regarding received neural data from the input controller;

whereby the input controller and the output controller form a second control loop having a shorter response latency than the first control loop.

Example 563 is the method of example 562, wherein the output controller carries out real time modulation of any parameters of the determined output signal sent to the at least one output device based at least in part on the selected data characterizing the received neural data.

Example 564 is the method of example 563, wherein the real time modulation happens in 1 to 100 microseconds.

Example 565 is the method of any one of examples 562 to 564, wherein the output controller receives selected data regarding timing of received neural data from the input controller;

wherein the output controller controls the time at which the determined output signal is sent based at least in part on the selected data regarding timing of received neural data.

Example 566 is the method of any one of examples 563 to 565, wherein the output controller receives selected data regarding amplitude of received neural data from the input controller;

wherein the output controller controls the amplitude and/or frequency of the determined output signal sent to the at least one output device based at least in part on the selected data regarding amplitude of received neural data.

Example 567 is the method of any of examples 550 to 566, wherein the output signal is a control signal of an end effector device.

Example 568 is the method of any of examples 550 to 567, wherein the output signal is a neural stimulation signal to be applied to the nervous system of the subject.

Example 569 is the method of any of examples 550 to 568, wherein the output signal is a control signal for an applied drug treatment.

Example 570 is the method of example 568, wherein the neural control method further comprises using an output controller to receive the determined output neural stimulation signal and to send the determined output neural stimulation signal to the at least one output device, and to send data regarding the timing of the determined output neural stimulation signal to the input controller.

Example 571 is the method of example 570, wherein the input controller stops sending the received neural data to the at least one machine learning means when the determined output neural stimulation signal is being output.

Example 572 is the method of any of examples 569 to 571, wherein the neural control method further comprises sending the received neural data to an update machine.

Example 573 is the method of any of examples 549 to 572, wherein the neural control method further comprises sending any combination of; the record of calculated neural biomarkers, output signals, recorded neural data, data from other sensors, or data representing bodily state to the update machine.

Example 574 is the method of any of examples 549 to 573, wherein the neural control method further comprises receiving updates of the at least one machine learning model.

Example 575 is the method of example 574, wherein the updates to the at least one machine learning model are calculated based on the received neural data calculated neural biomarkers, output signals, recorded neural data, data from other sensors, or data representing bodily state.

Example 576 is the method of any of examples 549 to 575, wherein the neural control method further comprises receiving updates to the ideal bodily setpoint or calculation of bodily setpoint as a means of updating the applied treatment.

Example 577 is the method of any of examples 549 to 576, wherein the neural control method further comprises receiving updates to the body model.

Example 578 is the method of examples 574 to 577 wherein the updates are generated based on data recorded during specific periods of guided activity during rehabilitation or recalibration periods.

Example 579 is the method of example 574 to 578 wherein the update machine is an automated cloud method.

Example 580 is the method of example 574 to 578 wherein the update machine is a manual connection over a local wired or wireless connection.

Example 581 is the method of examples 574 to 578 wherein the updates are automatically calculated by one or more machine learning methods for calculating long term treatment.

Example 582 is the method of examples 574 to 578 wherein the updates are chosen by a treating clinician.

Example 583 is the method of any of examples 549 to 582 wherein the neural control method comprises using a safety module to monitor performance of the neural control method.

Example 584 is the method of example 583 wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 585 is the method of example 583 or 584 wherein the safety module further monitors the performance of the real time input controller and output controller within the real time loop.

Example 586 is the method of examples 583 to 585 wherein the safety module is arranged to act to selectively reduce or stop the function of any element of the neural control method based on monitoring of performance of that element.

Example 587 is the method of examples 583 to 586 wherein the safety module is arranged to selectively control the neural control method to operate in a safety mode.

Example 588 is the method of examples 583 to 587, wherein the neural control method further comprises using an auditing module monitor version control of the neural control method; and

the safety module acts based on commands from the auditing module.

Example 589 is the method of any of examples 549 to 588, wherein the neural control method further comprises using an auditing module to monitor version control of the neural control method.

Example 590 is the method of example 588 or 589, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module is arranged to receive information from the update machine regarding the correct version of these elements.

Example 591 is the method of example 589 or 590, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control method of these elements.

Example 592 is the method of any one of examples 589 to 591, wherein the auditing module commands the safety module to act to reduce or stop the function of the neural control method based on the auditing module detecting at least one of:

Incorrect versioning

Out of date versioning

Unauthorised versioning

Example 593 is the method of any of examples 549 to 592 in which the at least one neural sensor operates using a first modality and the output signal is a neural stimulation signal to be applied to the nervous system of the subject using a second modality different from the first.

Example 594 is a neural control method comprising:

using an input controller to receive neural data regarding neural signals relating to a bodily state in nerves of a subject from at least one neural sensor;
using at least one neural data processing means to process the received neural data to determine at least one output signal required to achieve a desired value of the bodily state; and
using an output controller to receive the determined output signal and to send the determined output signal to the at least one output device;
whereby the neural control method forms a first control loop providing closed loop control of the bodily state;
wherein the output controller receives selected data regarding amplitude of received neural data from the input controller; and
wherein the output controller controls the amplitude of the determined output signal sent to the at least one output device based on the selected data regarding amplitude of received neural data.

Example 595 is the method of example 594, wherein the neural data processing means comprises a single machine learning model for processing the received neural data to directly determine the at least one output signal.

Example 596 is the method of example 594, wherein the neural data processing means comprises:

a first machine learning model for processing the received neural data to identify neural biomarkers; and
a second machine learning model to process the identified neural biomarkers and determine the at least one output signal.

Example 597 is the method of example 594, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 598 is the method of example 594, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 599 is the method of example 597 or 598, wherein the desired bodily setpoint is calculated within the closed loop controller based on received neural data or received neural biomarkers.

Example 600 is the method of example 598 or 599, wherein the neural control method uses the received data regarding bodily state of the subject from a non-neural sensor to calculate the desired bodily setpoint.

Example 601 is the method of example 597 or 598, wherein the closed loop controller uses the output of a body model to inform the decision of desired change in bodily state.

Example 602 is the method of example 601 wherein the body model is a state space model informed by any combination of neural biomarkers and/or non-neural sensors.

Example 603 is the method of example 601 wherein the body model is a functional model informed by any combination of neural biomarkers or non-neural sensors.

Example 604 is the method of examples 601 to 603 wherein the body model is updated based on received neural or other sensor data describing the bodily state of the subject subsequent to the applied output signal.

Example 605 is the method of example 604 wherein the body model update makes its own estimate of the response to the output signal and uses the comparison of this to the received neural or other sensor data to calculate the update to the body model.

Example 606 is the method of any of examples 594 to 605, wherein the neural control method further comprises receiving data regarding a bodily state of the subject from a non-neural sensor;

wherein the at least one machine learning means further processes the received data to determine the at least one output signal.

Example 607 is the method of any of examples 594 to 606, wherein the output controller receives selected data regarding received neural data from the input controller;

whereby the input controller and the output controller form a second control loop having a shorter response latency than the first control loop.

Example 608 is the method of example 607, wherein the output controller carries out real time modulation of any parameters of the determined output signal sent to the at least one output device based at least in part on the selected data characterizing the received neural data.

Example 609 is the method of example 608, wherein the real time modulation happens in 1 to 100 microseconds.

Example 610 is the method of any one of examples 607 to 609, wherein the output controller receives selected data regarding timing of received neural data from the input controller;

wherein the output controller controls the time at which the determined output signal is sent based at least in part on the selected data regarding timing of received neural data.

Example 611 is the method of any one of examples 607 to 609, wherein the output controller receives selected data regarding amplitude of received neural data from the input controller;

wherein the output controller controls the frequency of the determined output signal sent to the at least one output device based at least in part on the selected data regarding amplitude of received neural data.

Example 612 is the method of any of examples 594 to 611, wherein the output signal is a control signal of an end effector device.

Example 613 is the method of any of examples 594 to 612, wherein the output signal is a neural stimulation signal to be applied to the nervous system of the subject.

Example 614 is the method of any of examples 594 to 613, wherein the output signal is a control signal for an applied drug treatment.

Example 615 is the method of example 614, wherein the neural control method further comprises using an output controller to receive the determined output neural stimulation signal and to send the determined output neural stimulation signal to the at least one output device, and to send data regarding the timing of the determined output neural stimulation signal to the input controller.

Example 616 is the method of example 615, wherein the input controller stops sending the received neural data to the at least one machine learning means when the determined output neural stimulation signal is being output.

Example 617 is the method of any of examples 594 to 616, wherein the neural control method further comprises sending the received neural data to an update machine.

Example 618 is the method of any of examples 594 to 617, wherein the neural control method further comprises sending any combination of; the record of calculated neural biomarkers, output signals, recorded neural data, data from other sensors, or data representing bodily state to the update machine.

Example 619 is the method of any of examples 594 to 618, wherein the neural control method further comprises receiving updates of the at least one machine learning model.

Example 620 is the method of example 619, wherein the updates to the at least one machine learning model are calculated based on the received neural data calculated neural biomarkers, output signals, recorded neural data, data from other sensors, or data representing bodily state.

Example 621 is the method of any of examples 594 to 620, wherein the neural control method further comprises receiving updates to the ideal bodily setpoint or calculation of bodily setpoint as a means of updating the applied treatment.

Example 622 is the method of any of examples 594 to 621, wherein the neural control method further comprises receiving updates to the body model.

Example 623 is the method of examples 618 to 622 wherein the updates are generated due to data recorded during specific periods of guided activity during rehabilitation or recalibration periods.

Example 624 is the method of example 618 to 622 wherein the update machine is an automated cloud method.

Example 625 is the method of example 618 to 622 wherein the update machine is a manual connection over a local wired or wireless connection.

Example 626 is the method of examples 618 to 622 wherein the updates are automatically calculated by one or more machine learning methods for calculating long term treatment.

Example 627 is the method of examples 618 to 622 wherein the updates are chosen by a treating clinician.

Example 628 is the method of any of examples 5944 to 627, wherein the neural control method further comprise using a safety module to monitor performance of the neural control method.

Example 629 is the method of example 628, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 630 is the method of example 628 or 629, wherein the safety module further monitors the performance of the real time input controller and output controller within the real time loop.

Example 631 is the method of examples 628 to 630, wherein the safety module acts to selectively reduce or stop the function of any element of the neural control method based on monitoring of performance of that element.

Example 632 is the method of examples 628 to 631, wherein the safety module selectively controls the neural control method to operate in a safety mode.

Example 633 is the method of examples 628 to 632, wherein the neural control method further comprises using an auditing module to monitor version control of the neural control method; and

the safety module is acts based on commands from the auditing module.

Example 634 is the method of any of examples 594 to 633, wherein the neural control method further comprises using an auditing module o monitor version control of the neural control method.

Example 635 is the method of example 633 or 634, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from the update machine regarding the correct version of these elements.

Example 636 is the method of example 634 or 635, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control method of these elements.

Example 637 is the method of any one of examples 634 to 636, wherein the auditing module commands the safety module to act to reduce or stop the function of the neural control method based on the auditing module detecting at least one of:

Incorrect versioning;

Out of date versioning;

Unauthorised versioning.

Example 638 is the method of example of any of examples 594 to 637 in which the at least one neural sensor operates using a first modality and the output signal is a neural stimulation signal to be applied to the nervous system of the subject using a second modality different from the first.

Example 639 is a neural control method comprising:

using an input controller to receive neural data regarding neural signals relating to a bodily state in nerves of a subject from at least one neural sensor;
using at least one neural data processing means to process the received neural data to determine at least one output neural stimulation signal required to achieve a desired value of the bodily state; and
using an output controller to receive the determined output neural stimulation signal and to send the determined output neural stimulation signal to the at least one output device;
whereby the neural control method forms a first control loop providing closed loop control of the bodily state;
wherein the output controller receives selected data regarding amplitude of received neural data from the input controller; and
wherein the output controller sends data regarding the timing of the determined output neural stimulation signal to the input controller; and

the input controller stops sending the received neural data to the at least one machine learning means when the determined output neural stimulation signal is being output.

Example 640 is the method of example 639, wherein the neural data processing means comprises a single machine learning model for processing the received neural data to directly determine the at least one output signal.

Example 641 is the method of example 639, wherein the neural data processing means comprises:

a first machine learning model for processing the received neural data to identify neural biomarkers; and
a second machine learning model to process the identified neural biomarkers and determine the at least one output signal.

Example 642 is the method of example 639, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 643 is the method of example 639, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 644 is the method of example 642 or 643, wherein the desired bodily setpoint is calculated within the closed loop controller based on received neural data or received neural biomarkers.

Example 645 is the method of example 643 or 644, wherein the neural control method uses the received data regarding bodily state of the subject from a non-neural sensor to calculate the desired bodily setpoint.

Example 646 is the method of example 642 or 643, wherein the closed loop controller uses the output of a body model to inform the decision of desired change in bodily state.

Example 647 is the method of example 646 wherein the body model is a state space model informed by any combination of neural biomarkers and/or non-neural sensors.

Example 648 is the method of example 646 wherein the body model is a functional model informed by any combination of neural biomarkers or non-neural sensors.

Example 649 is the method of examples 646 to 648 wherein the body model is updated based on received neural or other sensor data describing the bodily state of the subject subsequent to the applied output signal.

Example 650 is the method of example 649 wherein the body model update makes its own estimate of the response to the output signal and uses the comparison of this to the received neural or other sensor data to calculate the update to the body model.

Example 651 is the method of any of examples 639 to 650, wherein the neural control method further comprises receiving data regarding a bodily state of the subject from a non-neural sensor;

wherein the at least one machine learning means further processes the received data to determine the at least one output signal.

Example 652 is the method of any of examples 639 to 651, wherein the output controller receives selected data regarding received neural data from the input controller;

whereby the input controller and the output controller form a second control loop having a shorter response latency than the first control loop.

Example 653 is the method of example 652, wherein the output controller carries out real time modulation of any parameters of the determined output signal sent to the at least one output device based at least in part on the selected data characterizing the received neural data.

Example 654 is the method of example 653, wherein the real time modulation happens in 1 to 100 microseconds.

Example 655 is the method of any one of examples 652 to 654, wherein the output controller receives selected data regarding timing of received neural data from the input controller;

wherein the output controller controls the time at which the determined output signal is sent based at least in part on the selected data regarding timing of received neural data.

Example 656 is the method of any one of examples 652 to 654, wherein the output controller receives selected data regarding amplitude of received neural data from the input controller;

wherein the output controller controls the amplitude and/or frequency of the determined output signal sent to the at least one output device based at least in part on the selected data regarding amplitude of received neural data.

Example 657 is the method of any of examples 639 to 656, wherein the output signal is a control signal of an end effector device.

Example 658 is the method of any of examples 639 to 657, wherein the output signal is a neural stimulation signal to be applied to the nervous system of the subject.

Example 659 is the method of any of examples 639 to 658, wherein the output signal is a control signal for an applied drug treatment.

Example 660 is the method of example 658, wherein the output controller sends data regarding the timing of the determined output neural stimulation signal to the input controller.

Example 661 is the method of any of examples 639 to 660, wherein the neural control method further comprises sending the received neural data to an update machine.

Example 662 is the method of any of examples 639 to 661, wherein the neural control method further comprises sending any combination of; the record of calculated neural biomarkers, output signals, recorded neural data, data from other sensors, or data representing bodily state to the update machine.

Example 663 is the method of any of examples 639 to 662, wherein the neural control method further comprises receiving updates of the at least one machine learning model.

Example 664 is the method of example 663, wherein the updates to the at least one machine learning model are calculated based on the received neural data calculated neural biomarkers, output signals, recorded neural data, data from other sensors, or data representing bodily state.

Example 665 is the method of any of examples 639 to 664, wherein the neural control method further comprises receiving updates to the ideal bodily setpoint or calculation of bodily setpoint as a means of updating the applied treatment.

Example 666 is the method of any of examples 639 to 665, wherein the neural control method further comprises receiving updates to the body model.

Example 667 is the method of examples 663 to 666 wherein the updates are generated based on data recorded during specific periods of guided activity during rehabilitation or recalibration periods.

Example 668 is the method of example 663 to 667 wherein the update machine is an automated cloud method.

Example 669 is the method of example 663 to 667 wherein the update machine is a manual connection over a local wired or wireless connection.

Example 670 is the method of examples 663 to 667 wherein the updates are automatically calculated by one or more machine learning methods for calculating long term treatment.

Example 671 is the method of examples 663 to 667 wherein the updates are chosen by a treating clinician.

Example 672 is the method of any of examples 639 to 671, wherein the neural control method comprises using a safety module to monitor performance of the neural control method.

Example 673 is the method of example 672, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 674 is the method of example 672 or 673, wherein the safety module further monitors the performance of the real time input controller and output controller within the real time loop.

Example 675 is the method of examples 672 to 674, wherein the safety module is arranged to act to selectively reduce or stop the function of any element of the neural control method based on monitoring of performance of that element.

Example 676 is the method of examples 672 to 675, wherein the safety module is arranged to selectively control the neural control method to operate in a safety mode.

Example 677 is the method of examples 672 to 676, wherein the neural control method further comprises using an auditing module to monitor version control of the neural control method; and

the safety module to acts based on commands from the auditing module.

Example 678 is the method of any of examples 639 to 677, wherein the neural control method further comprises using an auditing module to monitor version control of the neural control method.

Example 679 is the method of example 677 or 678, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from the update machine regarding the correct version of these elements.

Example 680 is the method of example 678 or 679, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control method of these elements.

Example 681 is the method of any one of examples 678 to 680, wherein the auditing module is arranged to command the safety module to act to reduce or stop the function of the neural control method based on the auditing module detecting at least one of:

Incorrect versioning;

Out of date versioning;

Unauthorised versioning.

Example 682 is the method of example of any of examples 639 to 681 in which the at least one neural sensor operates using a first modality and the output signal is a neural stimulation signal to be applied to the nervous system of the subject using a second modality different from the first.

Example 683 is a neural control method comprising:

using an input controller to receive neural data regarding neural signals relating to a bodily state from at least one neural sensor;

using at least one machine learning means using at least one machine learning model to process the received neural data to determine at least one output signal required to achieve a desired value of the bodily state; and

sending the determined output signal to at least one output device;
whereby the neural control method forms a first control loop providing closed loop control of the bodily state; and
wherein the neural control method further comprises using an update machine, including sending the received neural data to the update machine;
wherein the update machine uses the received neural data to perform machine learning training to produce an updated neural control method; and
the neural control method further comprising replacing the at least one neural control method with the updated neural control method.

Example 684 is the method of example 683, wherein the uploaded neural data is used to retrain the machine learning means.

Example 685 is the method of example 683 or 684, wherein the at least one machine learning means is used to calculate neural biomarkers.

Example 686 is the method of examples 683 to 685, wherein the at least one machine learning means calculating neural biomarkers is updated based on a machine learning retraining from neural data representative of one or more patients across one or more time periods.

Example 687 is the method of examples 684 to 685, wherein the at least one machine learning model comprises a single machine learning model for processing the received neural data to directly determine the at least one output signal.

Example 688 is the method of examples 683 to 685, wherein the at least one machine learning model comprises:

a first machine learning model for processing the received neural data to identify neural biomarkers; and
a second machine learning model to process the identified neural biomarkers and determine the at least one output signal.

Example 689 is the method of examples 684 to 686, wherein the at least one machine learning model comprises:

a first machine learning model for processing the received neural data to identify neural biomarkers; and

a closed loop controller receiving the neural biomarkers describing bodily state alongside the desired bodily setpoint to determine the output signal.

Example 690 is the method of examples 683 to 685, wherein the at least one machine learning model comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 691 is the method of example 689 or 690, wherein the desired bodily setpoint is calculated within the closed loop controller based on received neural data or received neural biomarkers.

Example 692 is the method of example 690 or 691, wherein the neural control method uses the received data regarding bodily state of the subject from a non-neural sensor to calculate the desired bodily setpoint.

Example 693 is the method of example 689 or 690, wherein the closed loop controller uses the output of a body model to inform the decision of desired change in bodily state.

Example 694 is the method of example 693 wherein the body model is a state space model informed by any combination of neural biomarkers and/or non-neural sensors.

Example 695 is the method of example 693 wherein the body model is a functional model informed by any combination of neural biomarkers or non-neural sensors.

Example 696 is the method of example 693 to 269 wherein the body model is updated based on received neural or other sensor data describing the bodily state of the subject subsequent to the applied output signal.

Example 697 is the method of example 696 wherein the body model update makes its own estimate of the response to the output signal and uses the comparison of this to the received neural or other sensor data to calculate the update to the body model.

Example 698 is the method of examples 693 to 697, wherein the neural control method further comprises receiving data regarding a bodily state of the subject from a non-neural sensor;

wherein the at least one machine learning means further processes the received data to determine the at least one output signal.

Example 699 is the method of examples 683 to 698, wherein the neural control method further comprises using an output controller to receive the determined output signal and to send the determined output signal to the at least one output device.

Example 700 is the method of example 699, wherein the output controller is arranged to receive selected data regarding received neural data from the input controller;

whereby the input controller and the output controller form a second control loop having a shorter response latency than the first control loop.

Example 701 is the method of example 700, wherein the output controller carries out real time modulation of any parameters of the determined output signal sent to the at least one output device based at least in part on the selected data characterizing the received neural data.

Example 702 is the method of example 701, wherein the real time modulation happens in 1 to 100 microseconds.

Example 703 is the method of any one of examples 700 to 702, wherein the output controller receives selected data regarding timing of received neural data from the input controller;

wherein the output controller controls the time at which the determined output signal is sent based at least in part on the selected data regarding timing of received neural data.

Example 704 is the method of any one of examples 700 to 702, wherein the output controller receives selected data regarding amplitude of received neural data from the input controller;

wherein the output controller controls the amplitude and/or frequency of the determined output signal sent to the at least one output device based at least in part on the selected data regarding amplitude of received neural data.

Example 705 is the method of any of examples 683 to 704, wherein the output signal is a control signal of an end effector device.

Example 706 is the method of examples 683 to 705, wherein the output signal is a neural stimulation signal to be applied to the nervous system of the subject.

Example 707 is the method of any of examples 683 to 706, wherein the output signal is a control signal for an applied drug treatment.

Example 708 is the method of example 707, wherein the neural control method further comprises using an output controller to receive the determined output neural stimulation signal and to send the determined output neural stimulation signal to the at least one output device, and to send data regarding the timing of the determined output neural stimulation signal to the input controller.

Example 709 is the method of example 708, wherein the input controller stops sending the received neural data to the at least one machine learning means when the determined output neural stimulation signal is being output.

Example 710 is the method of any of examples 683 to 709, wherein the neural control method further comprises sending the received neural data to an update machine.

Example 711 is the method of any of examples 683 to 710, wherein the neural control method further comprises sending any combination of; the record of calculated neural biomarkers, output signals, recorded neural data, data from other sensors, or data representing bodily state to the update machine.

Example 712 is the method of any of examples 683 to 711, wherein the neural control method further comprises receiving updates of the at least one machine learning model.

Example 713 is the method of example 712, wherein the updates to the at least one machine learning model are calculated based on the received neural data calculated neural biomarkers, output signals, recorded neural data, data from other sensors, or data representing bodily state.

Example 714 is the method of any of examples 683 to 713, wherein the neural control method further comprises receiving updates to the ideal bodily setpoint or calculation of bodily setpoint as a means of updating the applied treatment.

Example 715 is the method of any of examples 693 to 697, wherein the neural control method further comprises means for receiving updates to the body model.

Example 716 is the method of examples 711 to 715 wherein the updates are generated based on data recorded during specific periods of guided activity during rehabilitation or recalibration periods.

Example 717 is the method of example 711 to 715 wherein the update machine is an automated cloud method.

Example 718 is the method of example 711 to 715 wherein the update machine is a manual connection over a local wired or wireless connection.

Example 719 is the method of examples 711 to 715 wherein the updates are automatically calculated by one or more machine learning methods for calculating long term treatment.

Example 720 is the method of examples 711 to 715 wherein the updates are chosen by a treating clinician.

Example 721 is the method of examples 683 to 720, wherein the neural control method comprises using a safety module to monitor performance of the neural control method.

Example 722 is the method of example 721, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 723 is the method of example 721 or 722, wherein the safety module further monitors the performance of the real time input controller and output controller within the real time loop.

Example 724 is the method of examples 721 to 723, wherein the safety module is arranged to act to selectively reduce or stop the function of any element of the neural control method based on monitoring of performance of that element.

Example 725 is the method of examples 721 to 724, wherein the safety module is arranged to selectively control the neural control method to operate in a safety mode.

Example 726 is the method of examples 721 to 725, wherein the neural control method further comprises using an auditing module to monitor version control of the neural control method; and the safety module acts based on commands from the auditing module.

Example 727 is the method of examples 683 to 716, wherein the neural control method further comprises using an auditing module to monitor version control of the neural control method.

Example 728 is the method of example 726 or 727, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from the update machine regarding the correct version of these elements.

Example 729 is the method of example 727 or 728, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control method of these elements.

Example 730 is the method of any one of examples 727 to 729, wherein the auditing commands the safety module to act to reduce or stop the function of the neural control method based on the auditing module detecting at least one of:

Incorrect versioning;

Out of date versioning;

Unauthorised versioning.

Example 731 is the method of examples 483 to 730, in which the at least one neural sensor operates using a first modality and the output signal is a neural stimulation signal to be applied to the nervous system of the subject using a second modality different from the first.

Example 732 is a neural control method comprising:

using an input controller to receive neural data regarding neural signals relating to a bodily state from at least one neural sensor;

using at least one neural data processing means to process the received neural data to determine at least one output signal required to achieve a desired value of the bodily state; and

sending the determined output signal to at least one output device;

whereby the neural control method forms a first control loop providing closed loop control of the bodily state; and

wherein the neural control method uses a safety module monitoring performance of the closed loop controller;

wherein the safety module acts to reduce or stop the function of any element of the first control loop based on monitoring its performance.

Example 733 is the method of example 732, wherein the safety module acts to reduce or stop the function of any element of the neural control system based on monitoring its performance.

Example 734 is the method of example 732 or 733, wherein the neural data processing means comprises a single machine learning model for processing the received neural data to directly determine the at least one output signal.

Example 735 is the method of examples 732 to 734, wherein the neural data processing means comprises:

a first machine learning model for processing the received neural data to identify neural biomarkers; and
a second machine learning model to process the identified neural biomarkers and determine the at least one output signal.

Example 736 is the method of examples 732 to 734, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 737 is the method of examples 732 to 734, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 738 is the method of example 736 or 737, wherein the desired bodily setpoint is calculated within the closed loop controller based on received neural data or received neural biomarkers.

Example 739 is the method of example 737 or 73 wherein the neural control method uses the received data regarding bodily state of the subject from a non-neural sensor to calculate the desired bodily setpoint.

Example 740 is the method of example 736 or 737, wherein the closed loop controller uses the output of a body model to inform the decision of desired change in bodily state.

Example 741 is the method of example 740 wherein the body model is a state space model informed by any combination of neural biomarkers and/or non-neural sensors.

Example 742 is the method of example 740 wherein the body model is a functional model informed by any combination of neural biomarkers or non-neural sensors.

Example 743 is the method of examples 740 to 742 wherein the body model is updated based on received neural or other sensor data describing the bodily state of the subject subsequent to the applied output signal.

Example 744 is the method of example 743 wherein the body model update makes its own estimate of the response to the output signal and uses the comparison of this to the received neural or other sensor data to calculate the update to the body model.

Example 745 is the method of examples 732 to 744, wherein the neural control method further comprises receiving data regarding a bodily state of the subject from a non-neural sensor;

wherein the at least one machine learning means further processes the received data to determine the at least one output signal.

Example 746 is the method of examples 732 to 745, wherein the neural control method further comprises using an output controller to receive the determined output signal and to send the determined output signal to the at least one output device.

Example 747 is the method of example 746, wherein the output controller receives selected data regarding received neural data from the input controller;

whereby the input controller and the output controller form a second control loop having a shorter response latency than the first control loop.

Example 748 is the method of example 747, wherein the output controller carries out real time modulation of any parameters of the determined output signal sent to the at least one output device based at least in part on the selected data characterizing the received neural data.

Example 749 is the method of example 748, wherein the real time modulation happens in 1 to 100 microseconds.

Example 750 is the method of any one of examples 747 to 749, wherein the output controller receives selected data regarding timing of received neural data from the input controller;

wherein the output controller controls the time at which the determined output signal is sent based at least in part on the selected data regarding timing of received neural data.

Example 751 is the method of any one of examples 747 to 749, wherein the output controller receives selected data regarding amplitude of received neural data from the input controller;

wherein the output controller controls the amplitude and/or frequency of the determined output signal sent to the at least one output device based at least in part on the selected data regarding amplitude of received neural data.

Example 752 is the method of examples 732 to 751, wherein the output signal is a control signal of an end effector device.

Example 753 is the method of examples 732 to 751, wherein the output signal is a neural stimulation signal to be applied to the nervous system of the subject.

Example 754 is the method of examples 732 to 752, wherein the output signal is a control signal for an applied drug treatment.

Example 755 is the method of example 754, wherein the neural control method further comprises using an output controller to receive the determined output neural stimulation signal and to send the determined output neural stimulation signal to the at least one output device, and to send data regarding the timing of the determined output neural stimulation signal to the input controller.

Example 756 is the method of example 755, wherein the input controller stops sending the received neural data to the at least one machine learning means when the determined output neural stimulation signal is being output.

Example 757 is the method of examples 732 to 756, wherein the neural control method further comprises sending the received neural data to an update machine.

Example 758 is the method of examples 732 to 757. wherein the neural control method further comprises sending any combination of; the record of calculated neural biomarkers, output signals, recorded neural data, data from other sensors, or data representing bodily state to the update machine.

Example 759 is the method of examples 732 to 758, wherein the neural control method further comprises receiving updates of the at least one machine learning model.

Example 760 is the method of example 759, wherein the updates to the at least one machine learning model are calculated based on the received neural data calculated neural biomarkers, output signals, recorded neural data, data from other sensors, or data representing bodily state.

Example 761 is the method of examples 732 to 760, wherein the neural control method further comprises receiving updates to the ideal bodily setpoint or calculation of bodily setpoint as a means of updating the applied treatment.

Example 762 is the method of any of examples 740 to 74 wherein the neural control method further comprises receiving updates to the body model.

Example 763 is the method of examples 758 to 762 wherein the updates are generated based on data recorded during specific periods of guided activity during rehabilitation or recalibration periods.

Example 764 is the method of example 758 to 762 wherein the update machine is an automated cloud method.

Example 765 is the method of example 758 to 762 wherein the update machine is a manual connection over a local wired or wireless connection.

Example 766 is the method of examples 758 to 762 wherein the updates are automatically calculated by one or more machine learning methods for calculating long term treatment.

Example 767 is the method of examples 758 to 762 wherein the updates are chosen by a treating clinician.

Example 768 is the method of example 732 to 767, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 769 is the method of example 768, wherein the safety module further monitors the performance of the real time input controller and output controller within the real time loop.

Example 770 is the method of examples 768 or 769, wherein the safety module acts to selectively reduce or stop the function of any element of the neural control method based on monitoring of performance of that element.

Example 771 is the method of examples 768 to 770, wherein the safety module selectively controls the neural control method to operate in a safety mode.

Example 772 is the method of examples 768 to 771, wherein the neural control method further comprises using an auditing module to monitor version control of the neural control method; and

the safety module acts based on commands from the auditing module.

Example 773 is the method of examples 732 to 772, wherein the neural control method further comprises using an auditing module to monitor version control of the neural control method.

Example 774 is the method of example 772 or 773, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from the update machine regarding the correct version of these elements.

Example 775 is the method of examples 772 to 774, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control method of these elements.

Example 776 is the method of any one of examples 772 to 775, wherein the auditing module is arranged to command the safety module to act to reduce or stop the function of the neural control method based on the auditing module detecting at least one of:

Incorrect versioning;

Out of date versioning;

Unauthorised versioning.

Example 777 is the method of examples 732 to 776, in which the at least one neural sensor operates using a first modality and the output signal is a neural stimulation signal to be applied to the nervous system of the subject using a second modality different from the first.

Example 778 is a neural control method comprising:

using an input controller to receive neural data regarding neural signals relating to a bodily state from at least one neural sensor;

using at least one neural data processing means to process the received neural data to determine at least one output signal required to achieve a desired value of the bodily state; and

sending the determined output signal to at least one output device;

whereby the neural control method forms a first control loop providing closed loop control of the bodily state; and

wherein the neural control method further comprises using an auditing module to monitor version control of the neural control system;

wherein the auditing module receives information from the update machine regarding the correct version of the neural control system;

wherein the auditing module obtains information regarding the current version of the neural control system; and

wherein the auditing module commands a safety module to act to reduce or stop the function of the neural control system based on the correct and current version of the neural control system.

Example 779 is the method of example 778, wherein the neural control method incorporates the safety module; and

the safety module is arranged for monitoring performance of the neural control method.

Example 780 is the method of example 779, wherein the auditing module is arranged to obtain information regarding the current version of the neural control method of the neural control method from the safety module.

Example 781 is the method of examples 778 to 780, wherein the neural data processing means comprises a single machine learning model for processing the received neural data to directly determine the at least one output signal.

Example 782 is the method of examples 778 to 780, wherein the neural data processing means comprises:

a first machine learning model for processing the received neural data to identify neural biomarkers; and
a second machine learning model to process the identified neural biomarkers and determine the at least one output signal.

Example 783 is the method of examples 778 to 780, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 784 is the method of examples 778 to 780, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 785 is the method of example 783 or 784, wherein the desired bodily setpoint is calculated within the closed loop controller based on received neural data or received neural biomarkers.

Example 786 is the method of example 784 or 785, wherein the neural control method uses the received data regarding bodily state of the subject from a non-neural sensor to calculate the desired bodily setpoint.

Example 787 is the method of example 783 or 784, wherein the closed loop controller uses the output of a body model to inform the decision of desired change in bodily state.

Example 788 is the method of example 787 wherein the body model is a state space model informed by any combination of neural biomarkers and/or non-neural sensors.

Example 789 is the method of example 787 wherein the body model is a functional model informed by any combination of neural biomarkers or non-neural sensors.

Example 790 is the method of examples 787 to 789 wherein the body model is updated based on received neural or other sensor data describing the bodily state of the subject subsequent to the applied output signal.

Example 791 is the method of example 790 wherein the body model update makes its own estimate of the response to the output signal and uses the comparison of this to the received neural or other sensor data to calculate the update to the body model.

Example 792 is the method of examples 778 to 790, wherein the neural control method further comprises receiving data regarding a bodily state of the subject from a non-neural sensor;

wherein the at least one machine learning means further processes the received data to determine the at least one output signal.

Example 793 is the method of examples 778 to 792, wherein the neural control method further comprises using an output controller to receive the determined output signal and to send the determined output signal to the at least one output device.

Example 794 is the method of example 793, wherein the output controller receives selected data regarding received neural data from the input controller;

whereby the input controller and the output controller form a second control loop having a shorter response latency than the first control loop.

Example 795 is the method of example 794, wherein the output controller carries out real time modulation of any parameters of the determined output signal sent to the at least one output device based at least in part on the selected data characterizing the received neural data.

Example 796 is the method of example 795, wherein the real time modulation happens in 1 to 100 microseconds.

Example 797 is the method of any one of examples 793 to 796, wherein the output controller receives selected data regarding timing of received neural data from the input controller;

wherein the output controller controls the time at which the determined output signal is sent based at least in part on the selected data regarding timing of received neural data.

Example 798 is the method of any one of examples 793 to 796, wherein the output controller receives selected data regarding amplitude of received neural data from the input controller;

wherein the output controller controls the amplitude and/or frequency of the determined output signal sent to the at least one output device based at least in part on the selected data regarding amplitude of received neural data.

Example 799 is the method of examples 778 to 798, wherein the output signal is a control signal of an end effector device.

Example 800 is the method of examples 778 to 799, wherein the output signal is a neural stimulation signal to be applied to the nervous system of the subject.

Example 801 is the method of examples 778 to 800, wherein the output signal is a control signal for an applied drug treatment.

Example 802 is the method of example 801, wherein the neural control method further comprises using an output controller to receive the determined output neural stimulation signal and to send the determined output neural stimulation signal to the at least one output device, and to send data regarding the timing of the determined output neural stimulation signal to the input controller.

Example 803 is the method of example 802, wherein the input controller is arranged to stop sending the received neural data to the at least one machine learning means when the determined output neural stimulation signal is being output.

Example 804 is the method of examples 778 to 803, wherein the neural control method further comprises sending the received neural data to an update machine.

Example 805 is the method of examples 778 to 804, wherein the neural control method further comprises sending any combination of; the record of calculated neural biomarkers, output signals, recorded neural data, data from other sensors, or data representing bodily state to the update machine.

Example 806 is the method of examples 778 to 805, wherein the neural control method further comprises receiving updates of the at least one machine learning model.

Example 807 is the method of example 806, wherein the updates to the at least one machine learning model are calculated based on the received neural data calculated neural biomarkers, output signals, recorded neural data, data from other sensors, or data representing bodily state.

Example 808 is the method of examples 778 to 807, wherein the neural control method further comprises receiving updates to the ideal bodily setpoint or calculation of bodily setpoint as a means of updating the applied treatment.

Example 809 is the method of any of examples 785 to 791, wherein the neural control method further comprises receiving updates to the body model.

Example 810 is the method of examples 805 to 809 wherein the updates are generated based on data recorded during specific periods of guided activity during rehabilitation or recalibration periods.

Example 811 is the method of example 805 to 809 wherein the update machine is an automated cloud method.

Example 812 is the method of example 805 to 809 wherein the update machine is a manual connection over a local wired or wireless connection.

Example 813 is the method of examples 805 to 809 wherein the updates are automatically calculated by one or more machine learning methods for calculating long term treatment.

Example 814 is the method of examples 805 to 809 wherein the updates are chosen by a treating clinician.

Example 815 is the method of example 779 or example 780, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 816 is the method of example 815, wherein the safety module further monitors the performance of the real time input controller and output controller within the real time loop.

Example 817 is the method of examples 815 or 816, wherein the safety module acts to selectively reduce or stop the function of any element of the neural control method based on monitoring of performance of that element.

Example 818 is the method of examples 815 to 817, wherein the safety module selectively controls the neural control method to operate in a safety mode.

Example 819 is the method of examples 815 to 818, wherein the safety module acts based on commands from the auditing module.

Example 820 is the method of example 778 to 819, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from the update machine regarding the correct version of these elements.

Example 821 is the method of example 778 to 820, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control method of these elements.

Example 822 is the method of examples 778 to 821, wherein the auditing module commands the safety module to act to reduce or stop the function of the neural control method based on the auditing module detecting at least one of:

Incorrect versioning;

Out of date versioning;

Unauthorised versioning.

Example 823 is the method of example of examples 778 to 822, in which the at least one neural sensor operates using a first modality and the output signal is a neural stimulation signal to be applied to the nervous system of the subject using a second modality different from the first.

Example 824 is a neural control method comprising:

using an input controller to receive neural data regarding neural signals relating to a bodily state from at least one neural sensor;

using at least one neural data processing means to process the received neural data to determine at least one output signal required to achieve a desired value of the bodily state; and

sending the determined output signal to at least one output device;
whereby the neural control method forms a first control loop providing closed loop control of the bodily state; and
wherein the desired value of bodily state is achieved by a bodily setpoint set in advance as a treatment.

Example 825 is the method of example 824, wherein the neural data processing means comprises a single machine learning model for processing the received neural data to directly determine the at least one output signal.

Example 826 is the method of example 824, wherein the neural data processing means comprises:

a first machine learning model for processing the received neural data to identify neural biomarkers; and
a second machine learning model to process the identified neural biomarkers and determine the at least one output signal.

Example 827 is the method of example 824, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 828 is the method of example 824, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 829 is the method of example 827 or 828 wherein the desired bodily setpoint is calculated within the closed loop controller based on received neural data or received neural biomarkers.

Example 830 is the method of example 828 or 829, wherein the neural control method uses the received data regarding bodily state of the subject from a non-neural sensor to calculate the desired bodily setpoint.

Example 831 is the method of example 827 or 828, wherein the closed loop controller uses the output of a body model to inform the decision of desired change in bodily state.

Example 832 is the method of example 831 wherein the body model is a state space model informed by any combination of neural biomarkers and/or non-neural sensors.

Example 833 is the method of example 831 wherein the body model is a functional model informed by any combination of neural biomarkers or non-neural sensors.

Example 834 is the method of examples 831 to 833 wherein the body model is updated based on received neural or other sensor data describing the bodily state of the subject subsequent to the applied output signal.

Example 835 is the method of example 834 wherein the body model update makes its own estimate of the response to the output signal and uses the comparison of this to the received neural or other sensor data to calculate the update to the body model.

Example 836 is the method of examples 834 to 835, wherein the neural control method further comprises means receiving data regarding a bodily state of the subject from a non-neural sensor;

wherein the at least one machine learning means further processes the received data to determine the at least one output signal.

Example 837 is the method of examples 825 to 836, wherein the neural control method further comprises using an output controller to receive the determined output signal and to send the determined output signal to the at least one output device.

Example 838 is the method of example 837, wherein the output controller receives selected data regarding received neural data from the input controller;

whereby the input controller and the output controller form a second control loop having a shorter response latency than the first control loop.

Example 839 is the method of example 838, wherein the output controller carries out real time modulation of any parameters of the determined output signal sent to the at least one output device based at least in part on the selected data characterizing the received neural data.

Example 840 is the method of example 839, wherein the real time modulation happens in 1 to 100 microseconds.

Example 841 is the method of any one of examples 837 to 840, wherein the output controller receives selected data regarding timing of received neural data from the input controller;

wherein the output controller controls the time at which the determined output signal is sent based at least in part on the selected data regarding timing of received neural data.

Example 842 is the method of any one of examples 837 to 840, wherein the output controller receives selected data regarding amplitude of received neural data from the input controller;

wherein the output controller controls the amplitude and/or frequency of the determined output signal sent to the at least one output device based at least in part on the selected data regarding amplitude of received neural data.

Example 843 is the method of examples 824 to 842, wherein the output signal is a control signal of an end effector device.

Example 844 is the method of examples 824 to 843, wherein the output signal is a neural stimulation signal to be applied to the nervous system of the subject.

Example 845 is the method of examples 824 to 844, wherein the output signal is a control signal for an applied drug treatment.

Example 846 is the method of example 845, wherein the neural control method further comprises using an output controller to receive the determined output neural stimulation signal and to send the determined output neural stimulation signal to the at least one output device, and to send data regarding the timing of the determined output neural stimulation signal to the input controller.

Example 847 is the method of example 846, wherein the input controller stops sending the received neural data to the at least one machine learning means when the determined output neural stimulation signal is being output.

Example 848 is the method of examples 824 to 847, wherein the neural control method further comprises sending the received neural data to an update machine.

Example 849 is the method of examples 824 to 848. wherein the neural control method further comprises sending any combination of; the record of calculated neural biomarkers, output signals, recorded neural data, data from other sensors, or data representing bodily state to the update machine.

Example 850 is the method of examples 824 to 849, wherein the neural control method further comprises receiving updates of the at least one machine learning model.

Example 851 is the method of example 850, wherein the updates to the at least one machine learning model are calculated based on the received neural data calculated neural biomarkers, output signals, recorded neural data, data from other sensors, or data representing bodily state.

Example 852 is the method of examples 824 to 851, wherein the neural control method further comprises receiving updates to the ideal bodily setpoint or calculation of bodily setpoint as a means of updating the applied treatment.

Example 853 is the method of any of examples 831 to 835, wherein the neural control method further comprises receiving updates to the body model.

Example 854 is the method of examples 851 to 853 wherein the updates are generated based on data recorded during specific periods of guided activity during rehabilitation or recalibration periods.

Example 855 is the method of example 849 to 853 wherein the update machine is an automated cloud system.

Example 856 is the method of example 849 to 853 wherein the update machine is a manual connection over a local wired or wireless connection.

Example 857 is the method of examples 849 to 853 wherein the updates are automatically calculated by one or more machine learning systems for calculating long term treatment.

Example 858 is the method of examples 849 to 853 wherein the updates are chosen by a treating clinician.

Example 859 is the method of examples 824 to 858, wherein the neural control method incorporates a safety module for monitoring performance of the neural control method.

Example 860 is the method of example 859, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 861 is the method of example 859 or 860, wherein the safety module further monitors the performance of the real time input controller and output controller within the real time loop.

Example 862 is the method of examples 859 to 861, wherein the safety module acts to selectively reduce or stop the function of any element of the neural control method based on monitoring of performance of that element.

Example 863 is the method of examples 859 to 862, wherein the safety module selectively controls the neural control method to operate in a safety mode.

Example 864 is the method of examples 859 to 863, wherein the neural control method further comprises using an auditing module to monitor version control of the neural control method; and

the safety module acts based on commands from the auditing module.

Example 865 is the method of examples 824 to 864, wherein the neural control method further comprises using an auditing module to monitor version control of the neural control method.

Example 866 is the method of example 864 or 865, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from the update machine regarding the correct version of these elements.

Example 867 is the method of example 865 or 866, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control method of these elements.

Example 868 is the method of any one of examples 865 to 866, wherein the auditing module commands the safety module to act to reduce or stop the function of the neural control method based on the auditing module detecting at least one of:

Incorrect versioning;

Out of date versioning;

Unauthorised versioning.

Example 869 is the method of examples 824 to 868 in which the at least one neural sensor operates using a first modality and the output signal is a neural stimulation signal to be applied to the nervous system of the subject using a second modality different from the first.

Example 870 is a neural control method comprising:

using an input controller to receive neural data regarding neural signals relating to a bodily state from at least one neural sensor;

using at least one neural data processing means to process the received neural data to determine at least one output signal required to achieve a desired value of the bodily state; and

sending the determined output signal to at least one output device;
whereby the neural control method forms a first control loop providing closed loop control of the bodily state;
wherein the desired value of bodily state is achieved by a bodily setpoint; and
wherein the setpoint is calculated within the closed loop controller based on received sensor data.

Example 871 is the method of example 870, wherein the neural data processing means comprises a single machine learning model for processing the received neural data to directly determine the at least one output signal.

Example 872 is the method of example 870, wherein the neural data processing means comprises:

a first machine learning model for processing the received neural data to identify neural biomarkers; and
a second machine learning model to process the identified neural biomarkers and determine the at least one output signal.

Example 873 is the method of example 870, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 874 is the method of example 870, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 875 is the method of example 873 or 874, wherein the desired bodily setpoint is calculated within the closed loop controller based on received neural data or received neural biomarkers.

Example 876 is the method of example 874 or 875, wherein the neural control method uses the received data regarding bodily state of the subject from a non-neural sensor to calculate the desired bodily setpoint.

Example 877 is the method of example 873 or 874, wherein the closed loop controller uses the output of a body model to inform the decision of desired change in bodily state.

Example 878 is the method of example 877 wherein the body model is a state space model informed by any combination of neural biomarkers and/or non-neural sensors.

Example 879 is the method of example 877 wherein the body model is a functional model informed by any combination of neural biomarkers or non-neural sensors.

Example 880 is the method of examples 877 to 879, wherein the body model is updated based on received neural or other sensor data describing the bodily state of the subject subsequent to the applied output signal.

Example 881 is the method of example 880 wherein the body model update makes its own estimate of the response to the output signal and uses the comparison of this to the received neural or other sensor data to calculate the update to the body model.

Example 882 is the method of examples 870 to 881, wherein the neural control method further comprises receiving data regarding a bodily state of the subject from a non-neural sensor;

wherein the at least one machine learning means further processes the received data to determine the at least one output signal.

Example 883 is the method of examples 870 to 882, wherein the neural control method further comprises using an output controller to receive the determined output signal and to send the determined output signal to the at least one output device.

Example 884 is the method of example 883, wherein the output controller receives selected data regarding received neural data from the input controller;

whereby the input controller and the output controller form a second control loop having a shorter response latency than the first control loop.

Example 885 is the method of example 884, wherein the output controller carries out real time modulation of any parameters of the determined output signal sent to the at least one output device based at least in part on the selected data characterizing the received neural data.

Example 886 is the method of example 885, wherein the real time modulation happens in 1 to 100 microseconds.

Example 887 is the method of any one of examples 884 to 886, wherein the output controller receives selected data regarding timing of received neural data from the input controller;

wherein the output controller controls the time at which the determined output signal is sent based at least in part on the selected data regarding timing of received neural data.

Example 888 is the method of any one of examples 884 to 887, wherein the output controller receives selected data regarding amplitude of received neural data from the input controller;

wherein the output controller controls the amplitude and/or frequency of the determined output signal sent to the at least one output device based at least in part on the selected data regarding amplitude of received neural data.

Example 889 is the method of examples 870 to 888, wherein the output signal is a control signal of an end effector device.

Example 890 is the method of examples 870 to 889, wherein the output signal is a neural stimulation signal to be applied to the nervous system of the subject.

Example 891 is the method of examples 870 to 890, wherein the output signal is a control signal for an applied drug treatment.

Example 892 is the method of example 891, wherein the neural control method further comprises using an output controller to receive the determined output neural stimulation signal and to send the determined output neural stimulation signal to the at least one output device, and to send data regarding the timing of the determined output neural stimulation signal to the input controller.

Example 893 is the method of example 892, wherein the input controller stops sending the received neural data to the at least one machine learning means when the determined output neural stimulation signal is being output.

Example 894 is the method of examples 870 to 893, wherein the neural control method further comprises sending the received neural data to an update machine.

Example 895 is the method of examples 870 to 894. wherein the neural control method further comprises sending any combination of; the record of calculated neural biomarkers, output signals, recorded neural data, data from other sensors, or data representing bodily state to the update machine.

Example 896 is the method of examples 870 to 895, wherein the neural control method further comprises receiving updates of the at least one machine learning model.

Example 897 is the method of example 896, wherein the updates to the at least one machine learning model are calculated based on the received neural data calculated neural biomarkers, output signals, recorded neural data, data from other sensors, or data representing bodily state.

Example 898 is the method of examples 870 to 897, wherein the neural control method further comprises receiving updates to the ideal bodily setpoint or calculation of bodily setpoint as a means of updating the applied treatment.

Example 899 is the method of any of examples 877 to 881, wherein the neural control method further comprises receiving updates to the body model.

Example 900 is the method of examples 895 to 899 wherein the updates are generated based on data recorded during specific periods of guided activity during rehabilitation or recalibration periods.

Example 901 is the method of example 895 to 899 wherein the update machine is an automated cloud system.

Example 902 is the method of example 895 to 899 wherein the update machine is a manual connection over a local wired or wireless connection.

Example 903 is the method of examples 895 to 899 wherein the updates are automatically calculated by one or more machine learning systems for calculating long term treatment.

Example 904 is the method of examples 895 to 899 wherein the updates are chosen by a treating clinician.

Example 905 is the method of examples 870 to 904, wherein the neural control method comprises using a safety module to monitor performance of the neural control method.

Example 906 is the method of example 905, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 907 is the method of example 905 or 906, wherein the safety module further monitors the performance of the real time input controller and output controller within the real time loop.

Example 908 is the method of examples 905 to 907, wherein the safety module acts to selectively reduce or stop the function of any element of the neural control method based on monitoring of performance of that element.

Example 909 is the method of examples 905 to 908, wherein the safety module selectively controls the neural control method to operate in a safety mode.

Example 910 is the method of examples 905 to 909, wherein the neural control method further comprises using an auditing module to monitor version control of the neural control method; and

the safety module acts based on commands from the auditing module.

Example 911 is the method of examples 870 to 909, wherein the neural control method further comprises using an auditing module to monitor version control of the neural control method.

Example 912 is the method of example 910 or 911, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from the update machine regarding the correct version of these elements.

Example 913 is the method of example 911 or 912, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control method of these elements.

Example 914 is the method of any one of examples 911 to 913, wherein the auditing module is commands the safety module to act to reduce or stop the function of the neural control method based on the auditing module detecting at least one of:

Incorrect versioning;

Out of date versioning;

Unauthorised versioning.

Example 915 is the method of examples 870 to 914 in which the at least one neural sensor operates using a first modality and the output signal is a neural stimulation signal to be applied to the nervous system of the subject using a second modality different from the first.

Example 916 is a neural control method comprising:

using an input controller to receive neural data regarding neural signals relating to a bodily state from at least one neural sensor;

using at least one neural data processing means to process the received neural data to determine at least one output signal required to achieve a desired value of the bodily state; and

sending the determined output signal to at least one output device;
whereby the neural control system forms a first control loop providing closed loop control of the bodily state; and
wherein the closed loop controller uses the output of a body model to inform the decision of desired change in bodily state;
wherein the current state of the body relative to the body model is informed by any combination of neural biomarkers or non-neural sensors.

Example 917 is the method of example 916, wherein the neural data processing means comprises a single machine learning model for processing the received neural data to directly determine the at least one output signal.

Example 918 is the method of example 916, wherein the neural data processing means comprises:

a first machine learning model for processing the received neural data to identify neural biomarkers; and
a second machine learning model to process the identified neural biomarkers and determine the at least one output signal.

Example 919 is the method of example 916, wherein the neural data processing means comprises:

a first machine learning model for processing the received neural data to identify neural biomarkers; and

a closed loop controller receiving the neural biomarkers describing bodily state alongside the desired bodily setpoint to determine the output signal.

Example 920 is the method of example 916, wherein the neural data processing means comprises:

a first machine learning model for processing the received neural data to identify neural biomarkers; and

a closed loop controller receiving the neural biomarkers describing bodily state alongside the desired bodily setpoint to determine the desired change in bodily state;

a second machine learning model to process the desired change in bodily state and determine the output signal.

Example 921 is the method of example 919 or 920, wherein the desired bodily setpoint is calculated within the closed loop controller based on received neural data or received neural biomarkers.

Example 922 is the method of example 920 or 921, wherein the neural control method uses the received data regarding bodily state of the subject from a non-neural sensor to calculate the desired bodily setpoint.

Example 923 is the method of examples 916 to 922, wherein the body model is a state space model informed by any combination of neural biomarkers and/or non-neural sensors.

Example 924 is the method of examples 916 to 922, wherein the body model is a functional model informed by any combination of neural biomarkers or non-neural sensors.

Example 925 is the method of examples 916 to 924, wherein the body model is updated based on received neural or other sensor data describing the bodily state of the subject subsequent to the applied output signal.

Example 926 is the method of example 925 wherein the body model update makes its own estimate of the response to the output signal and uses the comparison of this to the received neural or other sensor data to calculate the update to the body model.

Example 927 is the method of examples 916 to 926, wherein the neural control method further comprises receiving data regarding a bodily state of the subject from a non-neural sensor;

wherein the at least one machine learning means further processes the received data to determine the at least one output signal.

Example 928 is the method of examples 916 to 927, wherein the neural control method further comprises using an output controller to receive the determined output signal and to send the determined output signal to the at least one output device.

Example 929 is the method of example 928, wherein the output controller receives selected data regarding received neural data from the input controller;

whereby the input controller and the output controller form a second control loop having a shorter response latency than the first control loop.

Example 930 is the method of example 929, wherein the output controller carries out real time modulation of any parameters of the determined output signal sent to the at least one output device based at least in part on the selected data characterizing the received neural data.

Example 931 is the method of example 930, wherein the real time modulation happens in 1 to 100 microseconds.

Example 932 is the method of any one of examples 929 to 931, wherein the output controller receives selected data regarding timing of received neural data from the input controller;

wherein the output controller controls the time at which the determined output signal is sent based at least in part on the selected data regarding timing of received neural data.

Example 933 is the method of any one of examples 929 to 932, wherein the output controller receives selected data regarding amplitude of received neural data from the input controller;

wherein the output controller controls the amplitude and/or frequency of the determined output signal sent to the at least one output device based at least in part on the selected data regarding amplitude of received neural data.

Example 934 is the method of examples 916 to 933, wherein the output signal is a control signal of an end effector device.

Example 935 is the method of examples 916 to 934, wherein the output signal is a neural stimulation signal to be applied to the nervous system of the subject.

Example 936 is the method of examples 916 to 934, wherein the output signal is a control signal for an applied drug treatment.

Example 937 is the method of example 936, wherein the neural control method further comprises using an output controller to receive the determined output neural stimulation signal and to send the determined output neural stimulation signal to the at least one output device, and to send data regarding the timing of the determined output neural stimulation signal to the input controller.

Example 938 is the method of example 937, wherein the input controller stops sending the received neural data to the at least one machine learning means when the determined output neural stimulation signal is being output.

Example 939 is the method of examples 916 to 938, wherein the neural control method further comprises sending the received neural data to an update machine.

Example 940 is the method of examples 926 to 939, wherein the neural control method further comprises sending any combination of; the record of calculated neural biomarkers, output signals, recorded neural data, data from other sensors, or data representing bodily state to the update machine.

Example 941 is the method of examples 916 to 940, wherein the neural control method further comprises receiving updates of the at least one machine learning model.

Example 942 is the method of example 941, wherein the updates to the at least one machine learning model are calculated based on the received neural data calculated neural biomarkers, output signals, recorded neural data, data from other sensors, or data representing bodily state.

Example 943 is the method of examples 916 to 942, wherein the neural control method further comprises receiving updates to the ideal bodily setpoint or calculation of bodily setpoint as a means of updating the applied treatment.

Example 944 is the method of any of examples 916 to 943, wherein the neural control method further comprises receiving updates to the body model.

Example 945 is the method of examples 940 to 944 wherein the updates are generated based on data recorded during specific periods of guided activity during rehabilitation or recalibration periods.

Example 946 is the method of example 4940 to 944 wherein the update machine is an automated cloud system.

Example 947 is the method of example 940 to 944 wherein the update machine is a manual connection over a local wired or wireless connection.

Example 948 is the method of examples 940 to 944 wherein the updates are automatically calculated by one or more machine learning systems for calculating long term treatment.

Example 949 is the method of examples 940 to 944 wherein the updates are chosen by a treating clinician.

Example 950 is the method of examples 916 to 949, wherein the neural control method comprises using a safety module to monitor performance of the neural control method.

Example 951 is the method of example 950, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the performance of these elements is monitored by the safety module.

Example 952 is the method of example 950 or 951, wherein the safety module further monitors the performance of the real time input controller and output controller within the real time loop.

Example 953 is the method of examples 950 to 952, wherein the safety module acts to selectively reduce or stop the function of any element of the neural control method based on monitoring of performance of that element.

Example 954 is the method of examples 950 to 953, wherein the safety module selectively controls the neural control method to operate in a safety mode.

Example 955 is the method of examples 950 to 954, wherein the neural control method further comprises using an auditing module to monitor version control of the neural control method; and

the safety module is arranged to act based on commands from the auditing module.

Example 956 is the method of examples 956 to 955, wherein the neural control method further comprises using an auditing module arranged to monitor version control of the neural control method.

Example 957 is the method of example 495 or 956, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from the update machine regarding the correct version of these elements.

Example 958 is the method of example 956 or 957, wherein the neural control method comprises using one or more elements from the group of:

Machine learning neural processor;

Closed loop control processor;

Real time input controller;

Real time output controller;

Stimulation library selector;

Machine learning output processor;

Bodily setpoint calculator; and

Body model;

wherein the auditing module receives information from a safety module regarding the current version of the neural control method of these elements.

Example 959 is the method of any one of examples 495 to 958, wherein the auditing module commands the safety module to act to reduce or stop the function of the neural control method based on the auditing module detecting at least one of:

Incorrect versioning;

Out of date versioning;

Unauthorised versioning.

Example 960 is the method of examples 916 to 959 in which the at least one neural sensor operates using a first modality and the output signal is a neural stimulation signal to be applied to the nervous system of the subject using a second modality different from the first.

Example 961 is the system of any one of examples 1 to 480, wherein the neural signals are natural neural signals or evoked neural signals.

Example 962 is the system of one of examples 15 to 19, 82 to 86, 127 to 131, 172 to 176, 220 to 224, 267 to 271, 314 to 318, 358 to 362, 404 to 408, or 459 to 463, wherein the first control loop is activated for only a part of the time.

Example 963 is the system of example 962, wherein the first control loop is activated based, at least in part on values of the received neural data and/or other sensor data.

Example 964 is the system of example 961 or example 962, wherein the second control loop is activated substantially continuously.

Example 965 is the method of any one of examples 481 to 960, wherein the neural signals are natural neural signals or evoked neural signals.

Example 966 is the method of one of examples 495 to 499, 562 to 566, 607 to 611, 652 to 656, 700 to 704, 747 to 751, 794 to 798, 838 to 842, 884 to 888, or 929 to 963, wherein the first control loop is activated for only a part of the time.

Example 967 is the method of example 966, wherein the first control loop is activated based, at least in part on values of the received neural data and/or other sensor data.

Example 968 is the method of example 966 or example 967, wherein the second control loop is activated substantially continuously.

Example 969 is a computer program comprising instructions which, when executed on a processing device, causes the processing device to carry out a method according to any of examples 481 to 960 or 965 to 968.

1. A neural control system comprising:
an input controller arranged to receive neural data regarding neural signals relating to a bodily state of a subject from at least one neural sensor;
at least one machine learning means using at least one machine learning model to process the received neural data to determine at least one output signal required to achieve a desired value of the bodily state; and
means arranged to send the determined output signal to at least one output device;
whereby the neural control system forms a first control loop providing closed loop control of the bodily state.

2. The system of claim 1, wherein the at least one machine learning model comprises a single machine learning model for processing the received neural data to directly determine the at least one output signal.

3. The system of claim 1, wherein the at least one machine learning model comprises:
a first machine learning model for processing the received neural data to identify neural biomarkers; and
a second machine learning model to process the identified neural biomarkers and determine the at least one output signal.

4. The system of claim 1, wherein the at least one machine learning model comprises:
a first machine learning model for processing the received neural data to identify neural biomarkers; and
a closed loop controller receiving the neural biomarkers describing bodily state alongside the desired bodily setpoint to determine the output signal.

5. The system of claim 1, wherein the at least one machine learning model comprises:
a first machine learning model for processing the received neural data to identify neural biomarkers; and
a closed loop controller receiving the neural biomarkers describing bodily state alongside the desired bodily setpoint to determine the desired change in bodily state;
a second machine learning model to process the desired change in bodily state and determine the output signal.

6. The system of claim 4, wherein the desired bodily setpoint is calculated within the closed loop controller based on received neural data or received neural biomarkers.

7. The system of claim 5, wherein the neural control system uses the received data regarding bodily state of the subject from a non-neural sensor to calculate the desired bodily setpoint.

8. The system of claim 4, wherein the closed loop controller uses the output of a body model to inform the decision of desired change in bodily state.

9. The system of claim 8 wherein the body model is a state space model informed by any combination of neural biomarkers and/or non-neural sensors.

10. The system of claim 8 wherein the body model is a functional model informed by any combination of neural biomarkers or non-neural sensors.

11. The system of claim 8 wherein the body model is updated based on received neural or other sensor data describing the bodily state of the subject subsequent to the applied output signal.

12. The system of claim 11 wherein the body model update makes its own estimate of the response to the output signal and uses the comparison of this to the received neural or other sensor data to calculate the update to the body model.

13. The system of claim 1, wherein the neural control system further comprises means arranged to receive data regarding a bodily state of the subject from a non-neural sensor;
wherein the at least one machine learning means further processes the received data to determine the at least one output signal.

14. The system of claim 1, wherein the neural control system further comprises an output controller arranged to receive the determined output signal and to send the determined output signal to the at least one output device.

15. The system of claim 14, wherein the output controller is arranged to receive selected data regarding received neural data from the input controller;
whereby the input controller and the output controller form a second control loop having a shorter response latency than the first control loop.

16. The system of claim 15, wherein the output controller is arranged to carry out real time modulation of any parameters of the determined output signal sent to the at least one output device based at least in part on the selected data characterizing the received neural data.

17. The system of claim 16, wherein the real time modulation happens in 1 to 100 microseconds.

18. The system of claim 15, wherein the output controller is arranged to receive selected data regarding timing of received neural data from the input controller;
wherein the output controller is arranged to control the time at which the determined output signal is sent based at least in part on the selected data regarding timing of received neural data.

19-20. (canceled)

21. A neural control method comprising:
using an input controller to receive neural data regarding neural signals relating to a bodily state of a subject from at least one neural sensor;
using at least one machine learning model to process the received neural data to determine at least one output signal required to achieve a desired value of the bodily state; and
sending the determined output signal to at least one output device;
wherein the method forms a first control loop providing closed loop control of the bodily state.

22. A computer program comprising instructions which, when executed on a processing device, causes the processing device to carry out a method according to claim 21.
